(19) United States
(12) Patent Application Publication
(10) Pub. No.: US 2013/0309304 A1
(43) Pub. Date: Nov. 21, 2013

(54) COMPOUNDS AND METHODS

(71) Applicant: Bernardo Nadal-Ginard, Madrid (ES)

(72) Inventor: Bernardo Nadal-Ginard, Madrid (ES)

(21) Appl. No.: 13/836,102

(22) Filed: Mar. 15, 2013

Related U.S. Application Data
(63) Continuation-in-part of application No. 13/425,041, filed on Mar. 20, 2012.
Application No. 13/425,041 is a continuation of application No. 13/217,569, filed on Aug. 25, 2011, now abandoned.
Application No. 13/217,569 is a continuation of application No. 13/057,764, now abandoned.

(30) Foreign Application Priority Data
Aug. 5, 2008 (GB) 0814302.6

(51) Int. Cl. A61K 38/30; A61K 38/20; A61K 38/19; A61K 9/14; A61K 38/18

(52) U.S. Cl. CPC A61K 38/30; A61K 9/14; A61K 38/1833; A61K 38/1858; A61K 38/1825; A61K 38/18; A61K 38/1808; A61K 38/1841; A61K 38/196; A61K 38/185; A61K 38/2086; A61K 38/1875; A61K 38/19
USPC 424/489; 514/9.5; 514/8.5; 514/8.6; 514/8.2; 514/9.1; 514/7.6; 514/9.6; 514/8.1; 514/8.9; 514/7.8; 514/8.4; 424/85.2; 514/8.8; 514/8.3; 424/85.1; 435/375

(57) ABSTRACT

The present invention relates to pharmaceutical formulations suitable for targeting particular tissue and/or organ(s) with a formulated active ingredient, for example when administered upstream of the target organ or tissue, and to use of the same in treatment, methods of treatment administering the same and methods of preparing the formulations. The pharmaceutical formulations of the invention are for parenteral administration to a target tissue and comprise particles containing an active ingredient, and a biodegradable excipient, wherein 90% or more of the particles have a diameter of between 10 and 20 microns and the formulation is substantially free of particles with a diameter greater than 50 microns and less than 5 microns, such that where the formulation is administered upstream of the target tissue the ability of the active to pass through the target tissue and pass into systemic circulation is restricted. In one aspect, the pharmaceutical formulation comprises a hydrogel and one or more growth factors. In one aspect, the hydrogel is ureido-pyrimidinone (UPy). In one aspect, the growth factor is insulin-like growth factor-1 (IGF-1) and hepatocyte growth factor (HGF).

### CROSS-REFERENCE TO RELATED PATENT APPLICATIONS

This application is a Continuation-in-Part of U.S. application Ser. No. 13/425,041, filed Mar. 20, 2012, which is a Continuation of U.S. application Ser. No. 13/217,569, filed Aug. 25, 2011, which is a continuation of U.S. application Ser. No. 13/057,764 filed Feb. 6, 2011, which is a Continuation Application claiming priority of PCT Application No. PCT/EP2009/060171, filed Aug. 5, 2009, which claims priority from Great Britain application Serial No. 0814302.6, filed Aug. 5, 2008. Applicant claims the benefits of 35 U.S.C. §120 as to the PCT application and priority under U.S.C. §119 as to the said Great Britain application, and the entire disclosures of each of the above identified applications are incorporated herein by reference in their entireties.

The present disclosure relates to pharmaceutical formulations suitable for targeting particular tissue and/or organ(s) with a formulated active ingredient, for example when administered upstream of the target organ or tissue. The disclosure also relates to use of the same in treatment, methods of treatment administering the same and methods of preparing the formulations. In particular different growth factors and cytokines are employed to stimulate the intrinsic regenerative capacity of solid tissues by activating its resident stem cell population using a device, such as a catheter, for the localized delivery of the active compounds to the target tissue.

### BACKGROUND OF THE INVENTION

### Field of the Invention

Most medicines/pharmaceuticals are administered systemically, for example orally, intravenously, by vaccine, intramuscularly or the like. Notable exceptions are stents coated with active ingredients, certain respiratory formulations delivered directly to the lungs, certain radiotherapies which are directed to target areas and certain dermatological, ophthalmological, and otological treatments which are administered topically.

Nevertheless, when appropriate, it would be advantageous to be able to deliver the pharmaceutical primarily to a diseased tissue or organ, because this would reduce the dose required and also minimize side effects. Such an approach would be particularly advantageous for two main areas of medicine: a) the administration of growth factors and cytokines capable of activating the growth and differentiation of resident stem cells in a particular tissue. Because of the potent biological activity of these molecules, it would be desirable to limit their action to the intended tissue, with minimal or no spillover to the rest of the body; b) the delivery of cancer chemotherapeutic agents because if the cancerous tissue could be targeted specifically then it may allow the administration of higher doses to the targeted cells while minimizing the terrible toxic side effects of the same, at least to a significant extent.

In more acute situations such as in heart attacks and strokes better treatments may be possible, particularly those directed to regenerate the damaged tissue, if the organs affected could be specifically targeted. In chronic situations, such as Parkinson disease, diabetes, or pulmonary fibrosis, local administration of agents capable to reconstitute the deficient cell type(s) have the potential to improve the prognosis of the disease.

However, reproducible delivery of active ingredients to target tissue or a target organ in a therapeutically effective manner is influenced to a large extent on the components (including excipients) employed, their physical characteristics, the dose and the mode of delivery.

### BRIEF DESCRIPTION OF THE DRAWINGS

FIGS. 1(A-E) Show distribution and characterization of c-kit^{pos} cardiac cells in the adult porcine heart.

FIGS. 2(A-I) Show light microscopy images showing various expanded porcine cardiac cells

FIGS. 3(A-I) Show H&E staining of GF-treated porcine hearts

FIGS. 4(A-I) Show evidence of activation of endogenous CSCs

FIGS. 5(A-I) Show regenerating bands of small, newly formed cells

FIGS. 6(A-I) Show various images of newly formed tissue

FIG. 7 Shows an optical microscope image of PLGA particles with IGF-I prepared as per Example 1.

FIG. 8 Shows an electron micrograph of PLGA particles with IGF-I prepared as per Example 1.

FIGS. 9(A-B) Shows sections of porcine heart. Sections of the hearts of pig#1 (left image) and pig #2 (right image). The anterior wall of the left ventricle, irrigated by the left coronary artery, of pig #1 shows a number of microinfarcts (paler areas), while the myocardium of pig #2 is normal as shown by the uniform coloration.

FIGS. 10(A-D) show sections of porcine myocardium after administration of polystyrene microspheres or PLGA and growth factor microspheres. Sections of the myocardium of pig #3, sacrificed 30 min after the administration of a mixture of polystyrene (red beads-shown in the figure as grey, larger diameter, smooth circles) and PLGA+growth factors (green beads-shown in the figure as white, smaller diameter and more irregular shape) beads. The appearance difference in size between the red and green particles is due to the higher fluorescence of the red.

FIGS. 10(E-H) and 10(I-L) show sections of the myocardium of pig #4, sacrificed 24 hours after the administration of a mixture of polystyrene (red—shown in figures as grey, larger diameter, smooth circles) and PLGA+growth factors (green—shown in the figure as white, smaller diameter and more irregular shape) beads. The ratio of green to red beads is significantly lower in this animal because of the degradation of the PLGA microparticles. In FIGS. 10E, 10G, 10I, and 10K only red beads are detected, while in FIGS. 10F, 10H, 10J, and 10K the ratio is closer to 1:1.

FIGS. 11(A-B) show sections of porcine heart wherein endogenous cardiac stem cells are highlighted. Microscopic sections of two areas of pig #4. Myocytes are in grey. Nuclei in darker grey. The endogenous cardiac stem cells (CSCs) are identified by an arrow head (upper) and an arrow (lower). Their membrane is labeled in paler green. On the upper figure, the nuclei are clean because the cells are quiescent. On the lower figure all the CSCs have pale grey stain in the nuclei that identifies the protein Ki-67 a marker of cells that have entered the cell cycle.

FIGS. 12(A-D) show histological images of control and damaged quadriceps muscle.

FIG. 13A compares the effect in the number of regenerated cardiac myocytes in pigs post-AMI treated with a combination of two types of microspheres

FIG. 13B shows the left ventricle ejection fraction prior to, immediately after and 4 weeks post-AMI as determined by echocardiography of the pigs treated with different combinations of microspheres

FIGS. 14(A-C) show the Effects of the UPy hydrogel carrier on IGF-1/HGF release and bioactivity in vitro

FIGS. 15(A-H) show that UPy-IGF-1/HGF therapy improves cardiac function in chronic MI

FIGS. 16(A-J) show IGF-1/HGF treatment reduced pathological hypertrophy in the MI borderzone

FIGS. 17(A-E) show IGF-1/HGF administration leads to formation of new cardiac myocytes

FIGS. 18(A-C) show IGF-1/HGF leads to increased capillerisation and reduces microvascular resistance

FIGS. 19 (A-F) show IGF-1/HGF treatment increases the epCSC compartment and drives their cardiac commitment in Chronic MI

### ABBREVIATIONS AND ACRONYMS

CD45^{neg} CD45 negative
c-kit^{pos} c-kit positive
CSC cardiac stem/progenitor cell
CTRL control
EDV end diastolic volume
EF ejection fraction
epCSC endogenous porcine cardiac stem/progenitor cell
ESV end systolic volume
FAS fractional area shortening
GF growth factors IGF-1/HGF
HGF hepatocyte growth factor
IGF-1 insulin-like growth factor-1
LAD left anterior descending artery
LCx left circumflex artery
LV left ventricular
MI myocardial infarction
RT3DE real-time 3-dimensional echocardiography
UPy ureido-pyrimidinone moieties
UPy-GF growth factors IGF-1/HGF embedded in UPy hydrogel

The present disclosure provides a pharmaceutical formulation for parenteral, especially intra-arterial, administration to a target tissue comprising particles containing an active ingredient and a biodegradable polymer excipient, wherein 30% or more of the particles have a diameter of 25 microns or less and the formulation is substantially free of particles with a diameter greater than 50 microns, such that where the formulation is administered upstream of the target tissue the ability of the active ingredient to pass through the target tissue and pass into systemic circulation is restricted. That is to say the active ingredient is retained in the target tissue while its ability to pass through the target tissue and pass into systemic circulation is severely restricted or abolished. Thus, in a particular aspect of the invention a pharmaceutical formulation for parenteral administration to a cardiac tissue is provided, said pharmaceutical composition comprising particles containing an active ingredient and a biodegradable excipient, wherein 90% or more of the particles have a diameter of between 10 and 20 microns and the formulation is substantially free of particles with a diameter greater than 50 microns and less than 5 microns, such that where the formulation is administered upstream of the target tissue the ability of the active to pass through the target tissue and pass into systemic circulation is restricted. In one embodiment at least 90%, of the particles of the pharmaceutical invention have a diameter that is between 15 and 20 microns.

In an aspect of the invention a pharmaceutical formulation for parenteral, e.g. intra-arterial, administration to a cardiac tissue is provided, said pharmaceutical composition comprising particles containing an active ingredient, selected from the group consisting of HGF and IGF-I, and a biodegradable excipient, wherein 90% or more of the particles have a diameter of between 10 and 20 microns and the formulation is substantially free of particles with a diameter greater than 50 microns and less than 5 microns, such that where the formulation is administered upstream of the cardiac tissue the ability of the active to pass through the cardiac tissue and pass into systemic circulation is restricted.

Whilst not wishing to be bound by theory it is thought that formulations of the present disclosure, when administered in the arterial blood upstream of the target tissue or organ, are carried into the target tissue or organ by the circulation and due to the particle size and distribution lodge, in other words are trapped or caught in the capillaries in the tissue or organ, which are about 5-10 μm in diameter. Particles lodging in capillaries and blocking blood flow is not generally desirable but the number of capillaries affected by the formulation of the disclosure is relatively small, particularly as the formulation enables very low therapeutic doses to be employed. Furthermore, the biodegradable excipient melts, dissolves, degrades or in some way disassociates itself from the active and thus ultimately the “blockage” is removed. Thus the movement of the particle is restricted/retarded by lodging in capillaries, a reversible process which returns the capillaries back to the natural condition after a short period. Retarding the movement of the particle for a short period allows the active to be maintained in the vicinity of the target for an appropriate amount of time to facilitate local action or absorption of the active into the extravascular space of the tissue.

The formulation is designed such that most, if not all the active is released from the particle while immobilized in the target tissue vascular bed. Once the active load is released the particle is designed to be degraded and its constituent materials released into the general circulation to be either metabolized or eliminated through the liver and/or kidney.

The present disclosure provides a pharmaceutical formulation for parenteral administration to a target tissues comprising particles containing an active ingredient and a biodegradable excipient, wherein 30% or more of the particles have a diameter of 25 microns or less and the formulation is substantially free of particles with a diameter greater than 50 microns, such that where the formulation is administered upstream of the target tissue the active is retained in the target tissue or organ for a therapeutically effective period.

In particular the formulations of the present disclosure allow lower quantities of active ingredients to be employed because the majority of active is retained in the target tissue rather than being taken into the systemic circulation. This seems to increase the therapeutic window of the active. That is to say the dose range over which the ingredient is therapeutically active is increased allowing smaller absolute quantities to be administered. Local administration of a lower dose means that side effects are likely to be minimised.

Suitable doses are, for example in the range 0.05 μg/Kg to about 10 μg/Kg, such as 0.1 μg/Kg to about 0.5 μg/Kg, in particular 0.15, 0.2, 0.25, 0.35, 0.4 or 0.45 μg/Kg.

Administrating lower doses locally for therapeutic effect is particularly important for potent molecules, for example growth factors, which are known to have potential to stimulate oncogenesis. These potentially harmful side effects limit the utility of such molecules even though in the right circumstance they produce therapeutically beneficial effects.

The formulations of the present disclosure do not employ microspheres comprising a polystyrene, silica or other non-biodegradable bead with active ingredient attached thereto, because enduring resilient materials i.e. non-biodegradable materials such as polystyrene and silica may cause damage to local capillaries, and may act as foreign bodies and produce local inflammatory reactions. Moreover, such nonbiodegradable beads might eventually gain access to the systemic circulation and may then, for example accumulate in distant tissue such as the lungs and liver, all of which are undesirable.

Generally, each particle will comprise active and excipient. It is not intended that the description of the formulation refer to discrete particles of active and separate particles of biodegradable polymer in simple admixture.

Substantially free of particles over 50 microns as employed supra is intended to refer to formulations that meet the criteria to be administered as a parenteral formulation set down in the US pharmacopeia and/or European pharmacopeia.

In one embodiment substantially free may include containing less than 5% of said particles, particularly less than 1%, for example less than 0.5%, such as less than 0.1%.

In one embodiment the at least 50%, at least 60%, at least 70%, at least 75%, at least 80%, at least 85%, at least 90%, at least 95%, at least 98% such as at least 99% of the particles have a diameter of 25 microns or less.

In one embodiment the particle size is in the range 6 to 25 microns, such as 10 to 20 microns, particularly 15 or 20 microns, for example at least 50%, at least 60%, at least 70%, at least 75%, at least 80%, at least 85%, at least 90%, at least 95%, at least 98% such as at least 99% of the particles are the relevant size or within said range. Thus in one embodiment of the invention at least 95%, at least 98% or at least 99% of the particles of the pharmaceutical composition have a diameter of between 10 and 20 microns.

In another embodiment at least 95%, at least 98% or at least 99% of the particles of the pharmaceutical composition have a diameter of between 15 and 20 microns.

In one embodiment the formulation does not contain particles less than 1 micron in diameter.

In one embodiment the formulation does not contain particles less than 5 microns in diameter.

In one embodiment at least 30% of the particles with the active are retained in the target tissue after administration, for example at least 40%, at least 50%, at least 60%, at least 70%, such as at least 80% or more of the active particles are retained.

In one embodiment the active particle is retained in the target tissue or organ for a period in the range 5 minutes to 24 hours, for example 30 minutes to 5 hours, such as 1, 2, 3 or 4 hours.

The period that the formulation is retained in the relevant tissue or organ depends primarily on the excipient or the combination of excipients employed. Thus the properties required from the excipient in vivo are that:
it is biocompatible (i.e. generally non-toxic and suitable for administration to humans and/or animals),
within an appropriate time frame after administration it contributes to maintaining the particle integrity sufficiently for the particle movement to be retarded by, for example lodging in a capillary or arteriole in the target tissue or organ, and
it is biodegradable (that is to say it is capable of being processed or metabolised) by the body to release the active and after the active has been released.

Thus a biodegradable polymer excipient suitable for use in the present disclosure is a polymer or co-polymer that does not have a long residency time in vivo, ie would not include entities such a polystyrene, polypropylene, high density polyethene and material with similar properties. Biodegradable polymers must be non-toxic and broken down into non-toxic sub-units preferably locally, such that the amount of circulating fragments/debris from the excipient are minimised.

Suitable excipients can be found in the United States Pharmacopeia (USP) and include inorganic as well organic, natural and man-made polymers. Examples may include polymers such as polylactic acid, polygycolide or a combination of the same namely polylactic co-glycolic acid, polycaprolactone (which has a slower rate of biodegradation than polylactic co-glycolic acid), polyhydroxybutyrate or combinations thereof. Polyurethanes, polysaccharides, proteins and polyaminoacids, carbohydrates, kitosane, heparin, polyhyaluronic acid, etc may also be suitable The excipient is generally in the form of a particle, an approximate sphere (microsphere) to which the active can be attached or with which the active is associated or incorporated within.

Liposomes are not biodegradable polymer excipients within the meaning of the present disclosure. Liposomes are vesicles of a phospholipid bilayer generally comprising cholesterol. For diseases such as myocardical infarction induced by arterio sclerosis cholesterol levels are monitored as one of the risk factors for the disease and thus it may be advisable to avoid administering cholesterol containing formulations to such patients. In addition patients with liver cirrhosis may have increased difficulty metabolising lipids and dietary fats, therefore administration of liposomes to such patients may not be advisable.

In one embodiment the biodegradable excipient is not a hydrogel (a continuous phase of a corresponding colloidal dispersed phase).

Thus, both the rate of “release” of the active and the rate of “dissolution” of the particle can be altered by altering the excipient or/and the method of binding the active to the excipient, so for example employing polycaprolactone would provide a particle which takes longer to dissolve or disintegrate than a corresponding particle employing polylactic co-glycolic acid. If the active is embedded within the excipient it will be released more slowly than if it is on the surface of the particle. If on the surface and bound by electrostatic charge it will be released faster than if covalently bound. In one embodiment the excipient comprises polylactic co-glycolic acid.

In one embodiment substantially all the particles, for example 80, 85, 90, 95, 96, 97, 98, 99 or 100% of the particles comprise polylactic co-glycolic acid.

In one embodiment the polylactic co-glycolic acid is in the ratio 75:25 respectively. In one embodiment the excipient comprises two or more distinct polymers, the term polymer includes co-polymers.

In one embodiment the excipient may include an acrylate polymer, for example a methacrylate polymer.

In one embodiment the particle comprises alginate. In one embodiment the excipient comprises a biodegradable form of polyurethane.

In one embodiment the excipient is in the form of a microsphere. In one embodiment the disclosure employs a polyvinyl alcohol microsphere formulation. In one embodiment the microspheres are not albumin.

In one embodiment the active(s) employed are encapsulated within a biodegradable coating for example selected from the Eudragit range.

In one embodiment one or more active molecules are embedded within the particle.

For the active compounds to perform, as described in the present disclosure, they need to be administered into the circulation as a microparticle which because of its size, morphology and composition will travel with the blood flow to reach its target tissue. At the target, the particle should release its active load in a controllable manner.

To accomplish this goal, once unloaded, the particle should be degraded and its constituents either metabolized or delivered into the systemic circulation to be eliminated by the normal excretion systems of the body.

To accomplish these goals the microparticles should fulfill the following characteristics:

The microparticles should be of uniform size and morphology in order to insure that they reach and become lodged at the designed level of the circulatory system. Uniformity of size and shape is better controlled when the particles are spherical.

Most capillary beds allow free passage of particles with a diameter of <6 microns in diameter, the microspheres of this disclosure should have a diameter >6 microns, and preferably of ˜15 microns. Particles in the range of 20 microns in diameter or larger lodge into pre-capillary arterioles or arterioles and block the blood flow to several capillaries at once. Therefore, they might create microscopic infarctions. Thus for the delivery of regenerative therapies the most suitable diameter of the microspheres is in the range of 15 microns. In addition, however, particles having diameters of 5, 6, 7, 8, 9, 10, 11, 12, 13, 14, 16, 17, 18, 19, 20, 21, 22, 23, 24 and 25 are contemplated for use according to the present invention.

The time required to release the active compound once they have reached their target could range from minutes to days and even weeks, depending on the type of microsphere and the therapeutic goal.

The microspheres should be made with a biodegradable and non-toxic compound. The stability of the particle and its degradation time will depend on the composition and type of the microsphere. It might be designed to deliver its load before it starts degrading; alternatively it might be designed so that the delivery of its load occurs as the particle disintegrates.

The nature of the polymer used as excipient, its size, lability of the bonds between the monomers and degree of cross-linking, if any, will affect the rate of release of the active as well as the stability and degradability of the particle.

In all embodiments, the microspheres should be stable enough in solution for them not to substantially break or degrade during their administration into the circulation and the time required for them to reach the target vascular bed.

In a suitable embodiment of the disclosure, each particle will carry a single type of active compound. When a mixture of compounds is thought to be beneficial for therapeutic purposes, a mixture of microparticles, each loaded with a single type of compound, may be administered. This design simplifies the production of the therapeutic compounds and offers greater therapeutic flexibility, thereby allowing individualized medicaments to be prepared rapidly to meet the patient's individual specific needs.

In one embodiment a particle(s) employed has/have only one type of active molecule bound to it/them. In one embodiment a particle(s) employed has a mixture, such as two, three or four active molecules bound to it.

The active compound might be loaded onto the particle at the time of its formation and, for example be dispersed throughout the particle.

The active compound may be encapsulated inside the particle where the excipient forms the shell of the microsphere.

In one embodiment active(s) are bound to a particle(s) by covalent bonds, for example a polypeptide or protein is bonded to a microsphere through cross-linking by treatment with an aldehyde such as formaldehyde or glutaldehyde, for example by emulsifying the microsphere (or ingredient of the microspheres) in the presence of the active(s), a suitable aldehyde and homogenizing the mixture under conditions suitable for forming particles of the required size. Alternatively the active may be bonded to a carboxylate group located on the excipient microsphere.

In one embodiment the active(s) are bound to a particle(s) by electrostatic forces (charge). In one embodiment the active(s) are bound to a particle(s) through a polyelectrolyte such as, for example comprising sodium, potassium, magnesium and or calcium ions with chloride counter ions in aqueous solution.

In one embodiment the active(s) are bound to a particle(s) between layers of polyelectrolytes. The active compound may be loaded on the surface of the particle either by charge (electrostatic forces) or covalently bound. In one embodiment the active(s) is/are bound to the particle by electrostatic charge.

In one embodiment the active(s) is/are bound to the particle by polyelectolytes, for example by means of a polyelectrolyte shell covering the particle onto which the active attaches by charge.

The active compound may form a single layer on the surface of the particle or might be deposited in multiple layers either contiguous or separated by polyelectrolyte layers.

The active compound may be bound to the particle by means of “linkers” which on one hand bind to the excipient matrix and on the other to the active compound. These bonds might be either electrostatic or covalent.

The microparticles may for example be stabilized by lyophilization. Microparticle may also be stable when frozen.

In one embodiment the excipient is degraded rapidly in the range of minutes to hours, or over a longer period such as weeks to months. In one embodiment the formulation is such that once in the circulation one or more actives is/are rapidly released for example in period in the range of 1 to 30 minutes to about 1 to 12 hours.

In one embodiment the disclosure relates to a mixed population of particles that is to say, particles with different rates of “dissolution”, which may be used to provide a formulation with controlled or pulsed release.

Thus formulations of the disclosure can comprise particles with different release kinetics and degradation rates.

In one embodiment the active is released over a period of 1 to 24 hours. In one embodiment the active is released over a period of 1 day to 7 days.

Thus in one or more embodiments all the formulation of the disclosure is metabolized within 7 days of administration.

In one embodiment once in the circulation of the individual, the active(s) is/are released very slowly, over a period weeks to months, for example 1 week to 1, 2, or 3 months.

In one embodiment the population of particles is well characterized and for example has the same characteristics. That is to say the physical and/or chemical properties of each particle fall with a narrow defined range.

In one embodiment the size of the microspheres is monodispersed. Thus in one embodiment the particles of the formulation have mean particle size with a small standard deviation, for example at least 68% of particles have a size +/−1 micron of the mean, such as 99% of particles have a particle size +/−1 micron of the mean (eg 15+/−1 microns). In addition, compositions wherein the particles have at least 70%, at least 75%, at least 80%, at least 85%, at least 90%, at least 95%, at least 96%, at least 97%, or at least 98% of particles within +/−1 micron of the mean are contemplated by the present invention.

In one embodiment the formulation comprises a population of particles characterized in that the populations contains at least two distinct types of particle, for example the distinct particles may have different actives, coatings, particle size or a combination of the same.

In one embodiment the disclosure relates to a mixed population of particles comprising particles of active in admixture with particles of one or more further distinct actives.

It appears the particle size and distribution of the formulation influences the in v/vo profile of the formulation including how the formulation in distributed in the tissue. It seems that is insufficient to simply have a mean particle size within the range 10 to 20 microns because this allows some particles to have a much larger particle size and also a much smaller particle size. This variation can cause problems in vivo because, for example the small particles are not retained with the relevant tissue and the larger particles can damage the tissue. The amount of active:excipient employed may be in the ratio 1%:99% w/w, 5%:95% w/w, 10%:90% w/w, 20%:80% w/w, 30%:70% w/w, 40%:60% w/w, 50%:50% w/w, 60%:40% w/w, 70%:30% w/w, 80%:20% w/w or 90%:10% w/w, depending on what release profile is required. If the active is required to be release quickly or immediately in vivo a higher ratio of active to excipient may be chosen.

In one embodiment the microsphere employed has a half life of about 16 hours.

In one embodiment the formulation is lyophilized.

In another embodiment the formulation is frozen.

The particles of the disclosure are not magnetic to an appreciable extent. The active ingredient may be any medicine or pharmaceutical that may be administered in the form of a particle according to the disclosure.

In one embodiment 15×10⁶ particles (microspheres) are administered, such as 14×10⁶, 13×10⁶, 12×10⁶, 11×10⁶, 10×10⁶, 9×10⁶, 8×10⁶, 7×10⁶, 6×10⁶, 5×10⁶, 4×10⁶, 3×10⁶, 2×10⁶ or 1×10⁶ particles are administered.

A particle as employed herein may comprise, for example micronized drug, semisolid or hydrated entities such as proteins or biologically derived actives formulated as discrete particles provided the particle maintains its structure for a sufficient period to perform the required function. The disclosure also extends to particles with a liquid core provided that the external integrity of the particle is such that is can perform its function in vivo. The disclosure does not extend to particles with a gas core.

Microspheres may be fabricated by emulsifying a polymer solution, followed by evaporation of solvent. In other instances monomers are emulsified followed by thermal or UV polymerization. Alternatively, a polymer melt is emulsified and successively cooled to solidify the droplets. A size reduction of the emulsion can be obtained by homogenizing or sonicating the bulk. The microspheres can be collected by filtering and/or centrifuging the reaction mixture.

Biodegradable microspheres and microcapsules of biopolymers for the controlled release and targeted delivery of different pharmaceutical compounds and therapeutic macromolecules have been long known in a number of forms, particularly those of relatively large diameters as described in the present disclosure (see D. D. Lewis “Biodegradable polymers and drug delivery systems” M. Chasin and R. Langer, editors (Marcel Dekker, New York, 1990); J. P. McGee et al, J. Control. Release 34:77, 1995). Microspheres and microcapsules are routinely produced by mechanical-physical methods such as spraying constituent monomers into microdroplets of the size followed by either a drying or polymerization step. Such microparticles can also be formed through emulsification followed by removal of the emulsifying solvent (B. Miksa et al., Colloid Polym. Sci. 273: 47, 1995; G. Crotts et al., J. Control. Release 35:91, 1995). The main challenge of these methods is the production of a monodisperse population of particles in shape and size. This, for example can be achieved employing a technique of flow focusing in which a capillary nebulizer is used to form microdroplets of the proper size. In the process the components are submerged into a harvesting solution/solvent which serves to dissolve/suspend the microparticle components, followed by evaporation of the solvent to provide solidified microparticles.

This process may require that all the components of the microparticle be combined into a single mixture (the focused compound) from which are generated the microdroplets that will form the microparticles. As many of the polymers used for drug delivery are hydrophobic while most therapeutic macromolecules, and particularly proteins, are hydrophilic the mixture requires emulsifying to ensure a homogeneous composition is obtained before the microparticles are formed.

Alternatively particles may be prepared, for example by aspirating a solution of active into microspheres in a convection current, from a nozzle with a net electric charge toward a plate or entity with a counter charge, in an anode/cathode type arrangement.

In one embodiment particles employed have a net electric charge, for example a positive charge or negative charge. This may, for example assist the particle's movement being retarded in the target tissue or organ. This net charge may be balanced in the formulation for administration by counter ion spheres (for example without active) of a small dimension, for example less than 5 micron, which are not retained within the target tissue after administration.

In one embodiment the active ingredient is a biological molecule or derived therefrom, for example a protein such as an antibody or a growth factor, a cytokine or combination of entities.

In particular the formulations of the disclosure are, particularly useful for targeting/activating resident stems cells found in the relevant tissue.

In one preferred embodiment the disclosure is used to activate the resident stems, progenitors and/or precursors of a particular tissue or organ to stimulate regeneration of said tissue or organ.

In one embodiment the disclosure relates to localized administration of ligands for the receptors expressed by the stem cells present in the post-natal tissue for initiation of regeneration of the same. The ligand may, for example be a growth hormone as described herein.

In one embodiment the ligands are administered to activate the receptors present on the most undifferentiated stem cells present in each target tissue. These cells express the so-called “multipotency genes”, such as Oct 4, Sox2, Nanog, etc. and they have a potent regenerative capacity (hereafter known as Oct4-expressing stem cells).

In one embodiment the ligand is administered to the heart to minimize and/or regenerate tissue damage for example caused by myocardial infraction.

In one embodiment, the composition comprises supramolecular hydrogel.

In one embodiment, the composition comprises injectable supramolecular hydrogel.

In one embodiment, the composition comprises ureido-pyrimidinone (UPy).

In one embodiment, the growth factor is insulin-like growth factor-1 (IGF-1) and/or hepatocyte growth factor (HGF).

In one embodiment, the composition comprises ureido-pyrimidinone (UPy) and the growth factor is insulin-like growth factor-1 (IGF-1) and/or hepatocyte growth factor (HGF).

In one embodiment, the patient population to be treated using the present method and composition has myocardial infarction and has been operated for by-pass surgery.

In one embodiment, the present method comprises a targeted intramyocardial injection of a composition.

In one embodiment, the intramyocardial injection targets the borderzone of infarct scar.

In one embodiment, the method treats and prevents congestive heart failure.

In one embodiment, the present method treats chronic myocardial infarction.

In one embodiment, the present method activates c-kit^{pos}, CD45″g, and/or epCSCs.

In one embodiment, the present method increases c-kit^{pos}, CD45″g, and/or epCSCs population by four fold in the borderzone of treated hearts as compared to non-treated hearts.

In one embodiment, the present method reduces pathological cardiac hypertrophy, increases epCSC number and formation of new cardiomyocytes and capillaries.

In one embodiment, the composition comprises 0.1-0.4 μg, 0.4-0.8 μg, 0.8-1 μg, 1-2 μg, 2-4 μg, 4-8 μg or 8-10 μg of growth factor per ml of UPy hydrogel.

In one embodiment, the present method enhances myocardial repair and regeneration in the acute phase of myocardial infarction.

In one embodiment, the patient is not treated with composition comprising microspheres.

When an artery is obstructed the main effect is a loss of the tissue downstream from the obstruction. The specific consequence of the obstruction of a coronary artery is a myocardial infarction (MI) which results in the irreversible loss of a portion of the cardiac muscle. This loss results in a diminution of the contractile capacity of the myocardium and the pumping capacity of the heart which, when significant enough, limits its capacity to provide the appropriate cardiac output and produces a serious and progressive limitation of the person's capacity (reviewed in Nadal-Ginard et al., Circ. Res. 2003; 92:139).

In the USA and the EU alone over 1.5 million MIs are treated every year and there are over 11 million MI survivors (American Heart Association, 2007; British Heart Association, 2007). Of these, over 30% die during the first year post-infarct. The survival post-MI depends in large measure on the size of the infarct (% of muscle mass lost) due to the ischemic event. When the loss affects ˜40-45% of the left ventricular mass it produces an irreversible cardiogenic shock which is uniformly lethal (Page et al., 1971. N. Engl. J. Med. 285; 133). This segmental myocardial loss produces a reorganization of the reminder myocardium with increased cell death by apoptosis, hypertrophy of the surviving myocytes, increased fibrosis of the tissue and dilation of the ventricular chamber (Pfeffer, M. A. & Braunwald, E., 1990. Circulation 81:1161). This reorganization, known as “remodeling”, because of its negative effects on contractility, frequently evolves into cardiac failure (CF). After the first episode of CF post-MI the average survival is <5 years with a yearly mortality of ˜18% (American Heart Association, 2000).

Most or all the therapies to treat the loss of parenchymal tissue, due to ischemia or to other causes are directed to preserve or improve the function of the surviving tissue. In the case of an MI, all the therapies presently in use to treat the consequences of loss of cardiac contractile muscle are directed to preserve or enhance the contractile function of the surviving tissue and to reduce the continued loss of these muscle cells by apoptosis or by necrosis (see Anversa & Nadal-Ginard, 2002. Nature 415:240; Nadal-Ginard et al. 2003. Circ. Res. 92: 139). At present there is not a single approved therapy designed to regenerate or to replace the myocytes lost in the MI and, in this manner, restore the contractile function of the heart. Moreover, all the experimental approaches described until now are directed to improve the blood flow to the ischemic/necrotic area by stimulating the increase in the capillary network, most often by directly or indirectly delivering to the affected area growth factors such as vascular endothelial growth factor

(VEGF) either in protein form or in the form of cDNA. Not a single therapy is directed to the resident stem cells in the tissue to stimulate them to multiply and differentiate in order to regenerate together the parenchyma and microcirculation lost by the vascular accident.

The goal of the therapeutic approaches to the acute MI is to restore the blood flow the damaged muscle as soon as possible to prevent further muscle loss. These reperfusion therapies include the use of thrombolytic agents, balloon angioplasty or bypass surgery. In the USA in 1998 >500,000 angioplasties and a similar number of surgical bypasses were performed. These therapies often are successful in restoring blood flow to the ischemic muscle, but none are able to replace a single muscle cell already lost at the time of the intervention. If this loss has been substantial, the long term consequence is an inability to generate the required cardiac output which will inexorably evolve to terminal heart failure.

Until now the only option to effectively treat terminal heart failure has been cardiac transplant with all the medical (immunosuppressive therapy), logistic and economic problems that it entails. Even if these problems could be circumvented, the shortage of donors makes this therapy available to >1% of the patients in cardiac failure.

The formulations of the present disclosure allow the administration of the therapeutically active molecules to be administered in a form where the tissue or organ such as the heart can be targeted specifically to regenerate tissue, for example damaged by obstruction of an artery, by stimulating stem cells already present in the tissue to regenerate.

Stem cell therapy for tissue regeneration.—Recently some experimental approaches have been developed as alternatives to organ transplantation which are targeted to replace some of the cells lost by the organ or tissue of interest. These procedures have been modeled in the success of the bone marrow transplants carried out for over half a century. The capacity of a small population of cells in the bone marrow to generate all blood cell types, when transplanted in an immunologically competent individual, proved convincingly that adult tissues contained “stem cells” capable to generate and regenerated a tissue or a whole organ. This conceptual breakthrough has led to the developments of experimental approaches to repair damaged tissues using different types of stem cells isolated from the individual to be treated (autologous cell therapy) or isolated from an individual different from the one to receive them (heterologous cell therapy). These cells are either isolated on mass or first expanded in culture before being transplanted to produce the desired repair of the affected tissue. These cell therapy approaches take advantage of the natural regenerative properties of the stem cells for tissue regeneration.

The term “stem cell” is used here to identify a cell that has the properties of self-renewal (generate more cells like itself), is clonogenic (can be expanded starting from a single cell) and it is pluripotent; that is it can produce a progeny which will differentiate into different cell types, often present in the tissue where they reside. That is, the cells originated from a stem cell will acquire particular cellular specializations characteristic of the tissue or organ from which the stem cell originated or into which it is transplanted (Stem Cells: A Primer. 2000. National Institutes of Health USA).

The term “pluripotent” refers to cells which are capable of differentiating into a number of different cell types. In the context of this application the term “tretrapotent” refers to a cell that although it might not be totipotent (capable of generating a whole individual), it is capable to generate four different cell types; e.g. cardiomyocytes, vascular endothelial and smooth muscle cells and connective tissue fibroblasts.

The term “progenitor cell” refers to a descendant of a stem cell which has already committed to a particular differentiation pathway and, therefore, has a more restricted differentiation potential than the stem cell. The progenitor cell has a great capacity of amplification and, although it does not yet express markers of differentiation, it has the capacity to create a progeny that is more differentiated than itself. For example, the term may refer to an undifferentiated cell or to a cell that has differentiated to an extent short of its final differentiation. This cell is capable of proliferation and giving rise to more progenitor cells, therefore having the ability to generate a large number of mother cells that can in turn give rise to differentiated or differentiable daughter cells. In particular, the term progenitor cell refers to a generalized mother cell whose descendants (progeny) specialize, often in different directions, e.g., by acquiring completely individual characters, as occurs in progressive diversification of embryonic cells and tissues. A progenitor cell is more differentiated than a true stem cell because it has already restricted somewhat the multipotency of the stem cell from which it originated.

As used herein unless the context indicates to the contrary stem cell refers to stem cells, progenitor cells and/or precursor cells.

Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors as has been recently demonstrated with the iESCs (induced embryonic stem cells) (Takahashi et al., 2007. Cell 131:1-12).

A “precursor cell” is a descendant of the progenitor cell which has gone further down the differentiation pathway and has become committed to differentiate into a single cell type even though it might not yet express any of the identifiable markers for this cell type. The precursor cell is usually the one undergoing the last round of amplification before the appearance of the identifiable differentiated pheno type. Stem cells are present in the inner cell mass of the blastocyst, the genital ridges of the early embryo, the placenta and in the majority of tissues of the adult animals, including the human. In contrast to the stem cell derived from the inner cell mass of the blastocyst, in general, the stem cells isolated from adult tissues are a mixture of true stem cells, progenitors and precursors together with cells at the earliest stage of their final differentiation. Adult stem cells have now been identified in practically all tissues originated from each of the three embryonic cell layers (endoderm, mesoderm and ectoderm), ranging from the bone marrow, central and peripheral nervous system, all connective tissues, skin, gut, liver, heart, inner ear, etc.

It appears that these adult stems cells have regenerative capacity. Surprisingly, despite the high prevalence, severity and high economic costs of the ischemic cardiopathy in all developed countries, until recently there has been no search for procedures targeted to the regeneration of the adult myocardium. One of the reasons for this anomaly has been that until very recently the heart was considered a terminally differentiated organ without any intrinsic regenerative capacity of its contractile cells (MacLellan, W. R. & Schneider, M. D. 2000. Annu Rev. Physiol. 62:289; Reinlib. L. and Field, L. 2000. Circulation 101: 182; Pasumarthi, K. R. S, and Field, L J. 2002. Circ. Res. 90:1044; MacLellan, W. R. 2001. J. Mol. Cell. Cardiol. 34:87; Perin, E. C. et al 2003. Ciculation 107:935; see Anversa, P. and Nadal-Ginard, B. 2002. Nature 415:240; Nadal-Ginard, B. et al 2003 Circ. Res. 92:139). This concept was based on the experimentally well documented fact that in the adult heart the vast majority of cardiomyocytes are terminally differentiated and their capacity to re-enter the cell cycle has been irreversibly blocked. Thus, there is no doubt that these myocytes are not able to reproduce to generate new myocytes.

One consequence of the prevailing concept of the myocardium as a tissue without regenerative potential has been that all the so-called experimental “regenerative therapies” implemented until now have been based on the introduction within the damaged heart of different cell types that either are fetal myocytes or are believed to have some potential to differentiate into this cell type or into capillaries and microarterioles in order to substitute for the cells lost during the infarct. In this manner animal experiments have been performed transplanting fetal and adult skeletal muscle precursor cells, fetal cardiac myocytes, and embryonic stem cells either in their undifferentiated state or after their commitment to the cardiomyocyte pathway (Kocher et al., 2001. Nature Med. 7: 430).

With the exception of the skeletal muscle precursor (which are incapable of converting to cardiocytes and are unable to become electrically coupled to the myocardial cells) (Menasche et al., 2001. Lancet 357: 279; C Guo et al. 2007. J Thoracic and Cardiovasc Surgery 134:1332) which can be autologous, all other cell types listed are by necessity of heterologous origin and, therefore, have either to be accompanied by immunosuppressive therapy or the transplant is rapidly eliminated by the immune system. The fact is that none of these approaches have proved to be very effective in preclinical assays and all have many pitfalls.

One of the most intriguing characteristics of some of the adult stem cells is their “plasticity”. This property refers to the fact that when certain stem cells are placed within a tissue different from the one they originated from, they can adapt to this new environment and differentiate into the cell types characteristic of the host tissue instead of the donor tissue. Although the extent and nature of this plasticity for many cell types still remains controversial (Wagers & Weissman, 2004. CeIl 116:636-648; Balsam et al., 2004 Nature 428, 668-673; Murray et al, 2004. Nature 428, 664-668; Chien, 2004. Nature 428, 607-608), it has spawned countless preclinical protocols and clinical trials.

Among the adult stem cells described until now, those from the bone marrow have been the most studied and those that have shown a greater degree of “plasticity” (Kocher et al., 2001. Nature Med. 7: 430). Also widely used have been the so-called “mesenchymal stem cells” derived from adipose tissue (Rangappa, S. et al 2003. Ann Thorac Surg 75:775).

The capacity of bone marrow and adipose-tissue derived stem cells to re-populate damaged areas of different tissues and organs, the relative ease of their isolation, together with the earlier work of Asahara et al (1999; Circ. Res. 85: 221-228), has proven advantageous for the objectives of cell therapy to regenerate to cardiac muscle in experimental animals (Orlic et al, 2001. Nature 410:701; Orlic et al, 2001. Proc. Natl. Acad. Sci. USA 98: 10344; Nadal-Ginard et al, 2003. Circ. Res. 92:139;) and in the human (Tse et al., 2003. Lancet 361:47; Perin et al., 2003. Circulation 107:2294). Although it has been questioned by some, (Balsam, L. B. et al. 2004. Nature 428: 668; Murry, C. E. et al. 2004. Nature 428: 664), it is clear that bone marrow derived stem cells under certain conditions are capable to generated cardiomyocytes, capillaries and microarterioles, particularly when transplanted in the border area of an experimental myocardial infarction. (Quaini, F., et al., 2002. New Engl. J. Med. 346:5; Bayes-Geis, A. et al., 2003. Cardiovasc. Res. 56:404; Bayes-Genis, A. et al., 2004. Eur. J. Heart Fail. 6:399; Thiele, H. et al., 2004. Transplantation 77:1902). No similar information is available from the numerous clinical trials of cell therapy with either bone marrow- or adipose tissue-derived stem cells because no reliable histopathological data is available for evaluation. A major drawback of the techniques used for myocardial cell therapy is the complexity and inefficiency of the cell transplantation procedure itself. When the cells are transplanted through the coronary arterial tree, only 3-5% remains in the myocardium while the rest is spread throughout the body. If the cells are injected directly into the myocardium, it requires either a thoracotomy or the use of complex and time consuming instrumentation (Noga-type systems) in order to identify the target area. This technique requires specialized operators and it is only available in specialized medical centers. In addition, the intramyocardial injections, either by transendocardial (Noga) or transepicardial (surgical) route still delivers <50% of the cells to the tissue.

Without exception, all cell therapy approaches used up to the present time to produce myocardial regeneration post-myocardial infarction either in experimental animals or in the human have been developed completely ignoring the fact that the myocardium has an intrinsic regenerative capacity represented by its resident stem cells (Nadal-Ginard, B., at al., 2003. J. Clin. Invest. 111:1457; Beltrami et al, 2003. Cell 114:763-776; Torella, D., et al, 2004. Circ. Res. 94:514; Mendez-Ferrer, S. et al., 2006. Nature Clin. Prac. Cardiovasc. Med. 3 Suppl 1:S83; Torella et al, 2007. Cell. Mol. Life. Sci. 64:661).

As indicated above, until recently the accepted paradigm considered the adult mammalian heart as a post-mitotic organ without regenerative capacity. Although over the past few years this concept has started to evolve, all the experimental and clinical approaches to myocardial regeneration have continued to be based on the old dogma. For this reason all cardiac regeneration protocols have been based on cell transplantation in order to provide the myocardium with cells with regenerative potential.

It now seems that when formulations of the present disclosure are administered under appropriate conditions that the intrinsic regenerative capacity of the “stem cells” resident in the tissue or organ (such as the heart) can be stimulated or activated to regenerate the tissue or organ.

Thus in one aspect the disclosure provides a method for the regeneration of solid tissues in living mammals, including humans, which include the local delivery of ligands for the receptors expressed by the stem cells present in the post-natal tissue to be regenerated. These are cells that when stimulated physiologically or pharmacologically multiply in situ and differentiate into the parenchymal cells characteristics of the tissue or organ that harbors them.

New cardiomyocyte formation has been detected in both the normal heart and in pathological conditions such as MI and cardiac failure (Beltrami, A. P. et al., 2001. New Engl. J. Med. 344:1750; Urbanek, K. et al, 2003. Proc. Netl. Acad. Sci. USA. 100: 10440; Nadal-Ginard, B. et al., 2003. J. Clin. Invest. 111:1457; Nadal-Ginard, B. et al., 2003. Circ. Res. 92:139).

Interestingly, these new myocytes are significantly more abundant at the border zone of MIs where they are an order of magnitude more abundant than in the myocardium of age matched healthy individuals. These observations suggested that the adult human myocardium has the capacity to respond to acute and chronic increases in cell death with an abortive regenerative process that attempts to replace the dead myocytes (Anversa, P. & Nadal-Ginard, B. 2002. Nature 415: 240; Anvrsa, P. and Nadal-Ginard, B. 2002. New Engl. J. Med. 346:1410; Nadal-Ginard, B. et al., 2003. Circ. Res. 92:139).

Adult cardiac stem cells (CSCs) were first described in 2003 (Beltrami et al. 2003. Cell 114:763-776) and confirmed by several authors in the same and other species (see Torella, D., et al., 2004. Circ. Res. 94:514; Mendez-Ferrer, S. et al., 2006. Nature Clin. Prac. Cardiovasc. Med. 3 Suppl 1:S83; Torella et al., 2007. Cell. MoI. Life Sci. 64:661). These CSCs are self-renewing, clonogenic and multipotent because they give rise to cardiomyocytes, endothelial and smooth muscle vascular cells as well as to connective tissue fibroblasts. They were identified by expression of membrane markers associated with stem cells such as c-kit, the receptor for SCF, Sea I, MDR-I and IsI-I. It is now clear that the new myocytes formed in the adult heart are derived from the CSCs resident in the myocardium. These CSCs, when injected at the border of an infarct, have the capacity to regenerate the contractile cells and the micro vasculature lost as a consequence of a massive MI (Beltrami, et al, 2003. Cell: 114:763-776; Laugwitz, et al. 2005; Mendez-Ferrer et al., Torella et al., 2006; Torella et al., 2007).

In the heart of a healthy individual, almost all CSCs are in a resting state (Go) or cycling very slowly during the lifespan of the organism. At any given time, only a very small fraction of these cells is active, undergoing replication and differentiation just enough to replace the cells that die by wear and tear. In contrast, a large fraction of the CSCs—sometimes the majority—is activated in response to a physiological or pathological stress. In general, there is a direct correlation between the magnitude of the stress and the number of CSCs that became activated in response. This number of activated CSCs is in turn also directly correlated to the number of new myocardial cells generated. This response, which occurs from mouse to human (Nadal-Ginard, B. et al., 2003. Circ. Res. 92:139), reveals the existence of a biochemical pathway triggered by the stress that results in the activation of the CSCs.

The communication between the resident stem cells and their environment, at least in the myocardium, is regulated by a feed-back loop between the cardiomyocytes, that sense the changes in wall stress produced by increased physiological or pathological demands in cardiac output, and the stem cells responsible to produce an increase in muscle mass through the generation of new contractile cells and microcirculation to nurture them. The myocytes have a stereotypical response to stress independently of whether it is physiological or pathological (Ellison et al., 2007. J. Biol. Chem. 282: 11397-11409). This response consists in rapidly activating expression and secretion of a large battery of growth factors and cytokines such as HGF (hepatocyte growth factor), IGF-I (insulin-like growth factor 1), PDGF-β (platelet-derived growth factor 13), a family of FGFs (fibroblast growth factor), SDF-I (stromal cell-derived factor 1), VEGF (vascular endothelial growth factor), erythropoietin (EPO), epidermal growth factor (EGF), activin A and TGF β (transforming growth factor β), WINT3A and neurogeulin among others. This secretory response, in addition to stimulate the hypertrophy of the myocytes themselves through an auto/paracrine loop, also triggers the activation of CSCs in their vicinity because these cells express receptors for these myocyte-secreted factors and respond to them. This response activates genetic pathways downstream of the receptor that are responsible for cell survival, multiplication and differentiation. In addition, the activation of these receptors also activate a feed-back loop in the CSCs themselves which stimulates the production of the respective ligand by the CSCs, thus putting in place a self-sustained response which, in response to a single stimulus, can remain active for several weeks or until the increased mass produced has restored the myocardial wall stress to normal levels. Therefore, the CSCs respond to a paracrine stimulus with an auto/paracrine response which allows the maintenance of a sustained response to a short lived stimulation. Thus, normal cardiac cellular homeostasis is maintained through a continuous feed-back between myocytes and CSCs to produce and maintain the appropriate contractile muscle mass required to generated the needed blood cardiac output. The myocytes, which are unable to divide, depend on the CSCs to maintain or increase their cell number and the capillary density to guaranty their oxygen and nutrient supply. The CSCs, on the other hand, depend and respond to the biochemical cues produced by their surrounding myocytes to regulate their resting vs activated state.

In addition to the tissue-specific stem cells described above, we have recently found that the myocardium of mammals, including the human, as well as most other tissues, contain a small population of very undifferentiated cells that have many similarities to the embryonic stem cells (ESCs) which have been known for a long time to be multipotent; that is, a single cell is capable, when placed in the proper environment, to generate a whole organism identical to the one from which it originated. The main characteristic of these cells is their expression of a battery of so-called “multipotency genes” such as Oct4, Sox2, Nanog, etc (see U.S. provisional application Ser. No. 61/127,067) that confer multipotency to these cells, so that, independently of their tissue of origin they seem capable to give rise to most, if not all cell types of the body. In particular, Oct4-expressing cells isolated from the adult heart are capable to give raise to skeletal muscle, neurons, heart, liver, etc. Their regenerative capacity seems more robust and broader than that of the tissue-specific stem cells.

We believe that the Oct4-expressing cells are the origin of most, if not all, the tissue-specific stem cells of every organ and that their stimulation is the main source of the regenerative capacity of every individual tissue. Therefore, the stimulation of these cells is a primary target for the therapeutic approaches described herein.

Independently of their ability and/or efficiency to generate myocardial cells, when a large number of stem cells are introduced into a tissue, regardless of their tissue of origin, they have an important paracrine effect when transplanted into the myocardium and other tissues, as has been proven experimentally. The complex mixture of growth factors and cytokines produced by the transplanted cells have a potent anti-apoptotic effect over the cardiomyocytes and other cells in the area at risk and also in the activation of the endogenous stem cells that multiply and differentiate into muscle cells and micro vasculature. This paracrine effect starts very soon after the cell transplantation and can be documented in vitro.

It seems from the work performed in the examples herein that to stimulate the resident stem cells of a tissue (including the Oct4 expressing cells), in this case the myocardium, the growth factors and cytokines produced by the stressed myocytes and to which the CSCs respond could be as or more effective than cell transplantation to trigger a regenerative response. A combination of insulin-like growth factor 1 and hepatocyte growth factor may be particularly effective.

In one embodiment resident stems cell are activated, for example to stimulate regeneration of the tissue, to increase muscle density and/or cell function of target cells.

If the target cells are cardiac muscle then the increased function would, for example be greater/increase contractile function.

If the target cells are kidney cells, in a renal failure kidney patient, then the increased function may be increased capacity to generate EPO.

If the target cells are pancreatic cells then the increased function may be increased capacity to generate insulin.

It seems that formulations of the disclosure are able to stimulate/activate stems cells resident in “mature tissue” thereby obviating the need to administer “stem-cell” therapy to the patient as the resident stems are stimulated to undergo mitosis and grow.

Stimulating resident stems cells is distinct from angiogenesis. Angiogenesis is the process of stimulating growth of capillaries (which may be in tissue or tumors) (see Husnain, K. H. et al. 2004. J. MoI. Med. 82:539; Folkman, J., and D'Amore, P A. 1996. Cell 87:1153). In contrast, when formulations of the present disclosure employing appropriate ligands are administered a stem cells resident in the tissue, such as pluripotent cells, progenitor cells and/or a precursor cells are activated to generate new/additional tissue cells such as muscle cells.

All the regenerative approaches described until now have severe limitations either because of the nature of their biological target, the regenerative agent used and/or the route and mode of administration. The vast majority of so-called regenerative therapies have been directed to regenerate the capillary network of the ischemic myocardium using a variety of biological factors, such as vascular endothelial growth factor (VEGF), whose main role is to stimulate the growth of the surviving endothelial cells in the damaged tissue in order to expand the capillary network and improve the blood supply (Isner, J. M. and Losordo, D. W. 1999. Nature Medicine 5:491; Yamaguchi, J., et al, 2003. Circulation 107:1322; Henry, T. D., et al, 2003. Circulation 2003. 107:1359). These therapies neither attempt nor accomplish the regeneration of the parenchymal cells that perform the characteristic function of the tissue or organ; e.g. contractile cardiomyocytes in the heart, hepatocytes in the liver, insulin-producing 13 cells in the pancreas, etc. At best, these therapies have had modest effects and none of them has become part of standard medical practice. On the other hand, all the regenerative therapies designed to replace the functional cells of the tissue or organ have until now been based in the transplantation of cells believed to be able to take on the characteristics of the missing cells in the target tissue. These approaches are still in clinical trials. A main drawback for all the regenerative approaches used has been to deliver the regenerative agent to the damaged tissue and limit their spread throughout the rest of the body. This is a serious problem even when the regenerative agents are administered through the coronary arterial tree of the tissue to the treated. In the cases of myocardial cell therapy by coronary administration, only a very small fraction of the cells administered is retained in the heart, while the majority (>95%) rapidly enters the systemic circulation and it is distributed throughout the body. This also occurs when the regenerative agents are directly injected into the myocardium either trans-epicardially or trans-endocardially, as has been repeatedly demonstrated with the administration of a cell suspension. In addition, the trans-epicardial administration requires exposing the heart through a thoracotomy, while the trans-endocardial administration requires a sophisticated, time consuming and expensive procedure to map the endocardium to identify the regions suitable for injection (a Noga-type instrument), a procedure available in a very limited number of centers and the participation of an expert manipulator. In both cases, at best 50% of the administered compound is retained in the damaged are while the remainder is spread either throughout the thoracic cavity or through the systemic circulation. The formulations of the disclosure may be used in combination with the delivery of stems cells to a target tissue or organ and increase the number that are retained locally in comparison to other delivery mechanisms.

However, this disclosure describes a novel method to regenerate the parenchymal cells (that is, the functional, “noble” cells) of a tissue or organ that is based neither on cell transplantation nor on the growth stimulation of the surviving endothelial cells in order to improve the blood supply to the tissue or organ of interest. Instead, the methods described here are based in the stimulation in situ, that is, within the tissue, of the resident stem cells of such tissue by means of local delivery of specific growth factors and/or cytokines which are able to stimulate their activation, replication and differentiation to generate the parenchymal cells lost as well as the microvasculature needed for their growth, survival and function. This is possible because most, if not all adult tissues mammalian tissues, including human tissue, contain resident stem cells which are capable, when properly stimulated, of regenerating the cell types which are specific to the tissue or organ, as well as the vascular and mesenchymal supporting cells which accompany them.

Because some of the regenerative agents that stimulate the stem cells are very active and might stimulate the growth and translocation of a variety of cells they interact with, among them latent neoplastic cells, the potential clinical application of many of these factors will require the administration of the smallest therapeutic doses in a very localized manner in order to, as much as possible, limit exposure to the cells that are to be regenerated. Thus, the more localized the administration the lower the doses required and lower the risk of undesired side effect due to stimulation of by-stander cells in the same or other organs. More specifically, the disclosure describes a new approach for the use of therapeutic doses of different growth factors administered and delivered locally, instead of systemically or tissue-wide, to produce the regeneration of specific areas of a solid tissue. Because the delivery of the active compound is localized to the damaged tissue, the therapeutic dose required is a minute fraction of what would be needed with other available delivery methods. The formulation of the disclosure is capable, among others applications, to regenerate the heart muscle and its microvasculature after a myocardial infarction and/or in chronic cardiac failure.

In one embodiment the formulation is administered at the border of the damaged tissue, for example at the border or an ischemic zone.

Suitable ligands for stems cells include growth factors such as those listed in Table 1

TABLE 1
Examples of suitable stem cell ligands of the invention

HGF (hepatocyte growth factor),
IGF (insulin-like growth factor) such as IGF-I,
PDGF (Piatelct-tkrrvcd growth factor) such as PDGF-β,
FGF (fibroblast growth factor) such as aFGF (FGF-I) or bFGF (FGF-2)
and FGF-4,
SDF-I (stromal cell-derived factor 1),
EGF (epidermal growth factor)
VEGF (vascular endothelial growth factor), erythropoietin (EPO),
TGF β (transforming growth factor
G-CSF (Granulocyte-colony stimulating factor),
GM-CSF (Granulocyte-macrophage colony stimulating factor),
Bone morphogenetic proteins (BMPs, BMP-2, BMP-4)
Activin A,
IL-6,
Neurotrophic for example NGF (Nerve growth factor), neuroregulin,
BDNF (brain-derived neurotrophic factor), NT-3 (ncurotrophin-3),
NT-4 (neurotrophin-4) and (neurotrophin-1), which is structurally
unrelated to NGF, BDNF, NT-3 and NT-4
TPO (Thrombopoietm)
GDF-8 (Myostatin), or
GDF9 (Growth differentiation faclor-9).
Periostin

In one embodiment the growth factor(s) employed is human.

In one embodiment the growth factor employed is selected from HGF, IGF (such as IGF-I and/or IGF-2) and FGF, in particular HGF and IGF-I. These factors appear to be particularly effective in stimulating resident stem cells.

Combinations of growth factors may also be employed and, for example may be selected from the above-identified list, such as HGF and IGF-I and optionally VEGF.

In one embodiment the formulation for regenerating/activating stems cells does not consist of VEGF as the only active but for example may comprise a combination of actives include VEGF.

Nevertheless the formulation is suitable for localized delivery of VEGF as angiogenesis factor.

In one embodiment the growth factor formulation is employed in combination with an angiogenesis factor, for example administered concomitantly or sequentially by the same route or a different route.

In one embodiment the formulation comprises a cytokine, for example selected from IL-1, IL-2, IL-6, IL-10, IL-17, IL-18 and/or interferon.

In one embodiment the formulation comprises combinations of actives, for example a growth factor and a cytokine.

In combination formulations then the dose of each active may, for example be the same dose employed when the active is administered alone. The components employed in the formulations and/or methods of the disclosure, especially biological type actives may be derived from natural origin.

In one embodiment a biological type active employed is prepared by recombinant DNA technology.

In one embodiment the active or actives administered may be peptide fragments of a biological molecule, with the desired therapeutic effect.

In one or more embodiments the molecules employed are mutants of a biological molecule (for example a ligand of a receptor) with the desired therapeutic effect having the same, higher or lower affinity for the corresponding biological molecule.

In one embodiment the substance(s)/active employed is an aptomer (a small RNA molecule that binds to a receptor instead of the natural ligand).

In one embodiment the substance/active employed is an antibody that recognizes and binds to a target receptor, and in particular has a suitable specificity and/or avidity for the same. Desirably the antibody has the required activity to upregulate the receptor or down regulates the receptor thereby either producing activation or blocking of the same, as appropriate.

In one embodiment the active is a diaquine, which is an artificial antibody molecule that recognizes and binds to two of the receptors of interest resulting in either the activation or blocking of one and/or the other.

In one embodiment the substance/active employed is a small molecule with a molecular weight <5,000 Daltons.

In one embodiment one or more actives employed may be of synthetic origin.

For the formulation disclosed herein to target the desired organ or tissue then the formulation should be administered upstream of the organ or tissue. That is to say should be introduced into the circulation such that the flow of blood carries the formulation into the desired tissue/organ.

The formulation can be introduced upstream of an organ such as the heart employing a suitable device such as a catheter. Other major organs can be reached in this way. Similarly whilst is it rare it is also possible to use catheters to gain access to the liver. In other instances the formulation may be introduced by strategic intra-arterial injection or by retrograde venous injection and/or cannular before the target tissue.

The formulation may also be administered by infusion or a pump driven delivery device such as a syringe pump, for example of the type employed in the administration of heparin or morphine or contrast agents during catheterization. A suitable flow rate may for example be 0.5 mL/min

The formulation might also be administered through the so-called perfusion catheters that allow slowing down the rate of blood flow downstream from the site of the injection with an intra-arterial balloon, while maintaining perfusion of the tissue through a second lumen of the catheter. In a particularly suitable embodiment the formulation is administered into an artery upstream of the target tissue or organ.

In one embodiment a catheter is used to deliver the formulation of the disclosure into the artery supplying the target tissue or organ. In particular, the formulation may be delivered exclusively (primarily or substantially) to the segmental artery that supplies the area of the tissue or organ.

In one embodiment the catheter employed is a balloon catheter.

In one embodiment the catheter carries a filter mesh at its distal end with a pore size sufficiently small to prevent or hinder the release of microparticle aggregates >50, 25 or 20 μm, as required. In one embodiment the target cells are the cardiac stem cells resident in the postnatal heart.

In one embodiment the regeneration obtained includes together or separately the regeneration of cardiomyocytes and vascular structures composed of capillaries (endothelial cells) and/or arterioles (endothelium and vascular smooth muscle cells). In one embodiment the regeneration is induced at any time after a myocardial infarction (MI) be it acute or chronic, for example 0.5, 1, 1.5, 2, 2.5, 3, 3.5, 4, 4.5, 5, 5.5, 6, 6.5, 7, 7.5, 8, 8.5, 9, 9.5, 10, 10.5, 11 up to 24 hours after an acute infarction.

In one embodiment the regeneration is induced in an individual with ischemic heart disease, with or without a myocardial infarction. In one embodiment the regeneration is induced in the hearts of individuals that have developed cardiac failure (CF) either acute or chronic.

In one embodiment the regeneration is induced in individuals with ischemic, infectious, degenerative or idiopathic cardiomyopathy.

In one embodiment the target cells are the stem cells resident in the endocrine pancreas (stem cells of the islands of Langerhans).

In one embodiment the regeneration is induced in an individual with diabetes.

In one embodiment the target cells are the neural stem cells of the central nervous system (CNS).

In one embodiment the target stem cells are the neural stem cells of the spinal cord.

In one embodiment the regeneration is induced in an individual with a spinal cord lesion.

In one embodiment the target cells are the stem cells of the substantia nigra of the brain, for example in an individual with Parkinson's disease. In one embodiment the regeneration is induced in an individual with a cerebral vascular accident (stroke).

Whilst not wishing to be bound by theory it is believed that the ligands employed in formulations of the disclosure are able to cross the blood brain barrier to treat strokes and the like. In addition, in cerebral vascular accident it is believed that the blood brain barrier becomes impaired and chemical entities can more readily pass through the barrier.

In one embodiment the target cells are the liver stem cells and for example the regeneration is induced in an individual with liver damage such as cirrhosis.

In one embodiment the target stem cells are the stem cells of the lung(s) and for example the regeneration is induced in a patient with lung damage, for example emphysema.

In one embodiment the target cells are the stem cells of the skeletal muscle and for example the regeneration is induced in an individual with a particular skeletal muscle deficit, such as osteoporosis or pagets disease. In one embodiment the target cells are the stem cells of the epithelium.

In one embodiment the target stem cells are the stem cell of the kidneys. Target cells as employed herein refers to the cells that are to be stimulated and which have the potential to provide the desired regeneration.

The formulation of the disclosure provides optimized parameters and materials to ensure accurate and/or reproducible dosing of the relevant active to the target tissue or organ.

In an alternative embodiment the formulations of the disclosure may be employed to treat solid tumors, by allowing local delivery of the antineoplastic to the tumor tissue, for example by intra-tumor injection. Actives suitable for the treatment of tumors include etoposide, cyclophosphamide, genistein, cisplatin, andriamycin, vindesine, mitoguazone, fluorouracil and paclitaxil.

In one embodiment the formulation is not for the treatment of cancer.

In one embodiment the invention is not administration directly into a tumor or tissue. The methods according to the disclosure may employ combinations of actives administered separately, for example concomitantly or sequentially, or formulated as one (one-pot) formulation.

Formulations of the disclosure may be administered as liquid solutions/suspension, for example in an isotonic carrier, for example as a buffered solution such as phosphate buffer, saline or glucose solution.

Formulations of the disclosure may optionally comprise one or more further excipients. The excipients should be suitable for administration to humans and/or animals.

In one embodiment the formulation comprises albumin in solution, which may for example stabilize the small quantities of active in the formulations, for example from 1% to 20% w/vol of albumin, such as human serum album, may be sufficient to achieve the required stabilization.

The disclosure also extends to use of as a formulation as defined herein for treatment, particularly for the treatment of myocardial infarction; ischemic heart disease; cardiac failure; ischemic, infectious, degenerative or idiopathic cardiomyopathy, sclerosis, cirrhosis, emphysema, diabetes and the like.

In one embodiment the disclosure relates to a formulation as described herein for use in treatment, particularly for treatment of an illness described above.

The disclosure also extends to methods of treatment comprising administering a therapeutically effective amount of a formulation described herein to a patient in need thereof, particularly for the treatment of a disease described above.

The disclosure also extends to use of a ligand, for example as described herein, for stimulating a resident stem cell in vivo to activate the cell.

The disclosure also includes uses of a suitable growth factor for the manufacture of a medicament for stimulate resident stem cells in vivo.

The disclosure will now be illustrated by reference to the Examples.

### EXAMPLES

### Introduction

Anterior myocardial infarctions were produced in female pigs by temporary balloon occlusion of the anterior descending coronary artery distal to the first septal branch. This procedure resulted in anterior-apical infarctions of reproducible moderate size. The myocardial regeneration potential of combined insulin-like growth factor 1 and hepatocyte growth factor was tested by locally administering the factors at different doses in the infarcted pig myocardium. Control animals were treated with placebo. The feasibility to produce therapeutic effects with local administration of minute amounts of therapeutic agents was tested first by direct administration of a solution containing a mixture of recombinant human IGF-I and HGF in the acute post-MI produced in an experimental model with closed chest by balloon dilation in the anterior descending left coronary artery just below the emergence of the first septal artery in 23 pigs that were compared to 6 placebo controls identically treated.

### Materials and Methods

The hearts were analyzed at different time points after myocardial infarction, ranging from a few days to 1 month. The results showed a dramatic increase in the number of activated stem and progenitor cells in the ischemic area and its borders of pigs treated with human IGF-I and HGF. Notable regeneration of the muscle was seen in the ischemic area, which also contained newly formed arterioles and vessels. The regenerative response seemed to be proportional to the doses of growth factors administered. From these preliminary data, therapeutic in situ activation of CSCs can produce extensive new myocardial tissue formation and significantly improve left ventricular function in animal hearts that are similar in size and anatomy to human hearts.

Isolation of c-kit^{pos} Porcine Cardiac Cells

Multiple cardiac samples (˜2 g each) were obtained from different cardiac regions (right and left atria, right and left ventricle and apex) of female Yorkshire white pigs (23±4 kg; n=3). Some samples were fixed and embedded in paraffin for histochemical analysis. The other pieces were enzymatically digested and cardiomyocyte-depleted cardiac cell suspensions were prepared as previously described with modifications (Beltrami, A. P. et al., 2003. Cell 114:763). Briefly, minced cardiac tissue was digested with 0.1% collagenase (Worthington Biochemicals), 0.1% Trypsin (Sigma), 0.1% DNAse I in Hanks' balanced salt solution (HBSS) buffer at 37° C. and the small cardiac cell fraction collected through centrifugation. Cardiac small cells were incubated with anti-human CD117(c-kit) Ab (Miltentyi Biotechnology) and sorted by fluorescence-activated cell sorting (FACS; MoFIo (Dako Cytomation) cell sorter) or magnetic activated micro-immunobeads (MACS). Propidium iodide (PI; 2 μg/mL) was added before FACS to exclude dead cells. c-kit^{pos} porcine cardiac cells were analysed for hematopoietic, mesenchymal and endothelial cell markers using a FacsCalibur flow cytometer (Becton Dickinson, BD). Antibodies used were anti-porcine CD45 (Serotec, Clon: MCA1447), anti-human CD34 (BD, clon 8G12), anti-human CD90, (BD, Clon:5E10, pig cross-reactivity) and anti-human CD 166 (BD, Clon: 3A6, pig cross-reactivity), anti-human CD 105 (Caltag Laboratories, Clon: SN6, pig cross-reactivity) and anti-human CD 133 (Miltenyi Biotec, clon AC 133, pig cross-reactivity). Anti-human antibodies specific to PECAM, E-cadherin, CD1Ib, CD13, CD14, CD29, CD31, CD33, CD36, CD38, CD44, CD49, CD62, CD71, CD73, CD106, were purchased from BD Biosciences. Respective isotype controls (Pharmingen) were used as negative controls for all FACS procedures. Data were analysed using the CellQuest software.

Porcine c-kit^{pos} Cardiac Cell Culture, Cloning, and Differentiation Potential c-kit^{pos} cells were plated for 7-10 days at 2×10⁴ cells/ml in Dulbecco's MEM/Ham's F12 (DMEM/F12) modified medium containing 10% FBS, bFGF (10 ng/ml), insulin-transferrin-selenite (ITS), and EPO (2.5 U). After recovery, some cells were moved to a modified cardiosphere formation media (mCSFM): 1:1 ratio of DMEM/F12, bFGF (10 ng/ml), EGF (20 ng/ml), ITS, 2-β-mercapethanol (0.1 mM) and Neural Basal Media supplemented with B27 and N2 supplements (Gibco), for the generation of cardiospheres. To test for clonogenicity, single c-kit^{pos} cells were seeded individually into wells of 96-well gelatin coated Terasaki plates by flow cytometry or serial dilution. Individual c-kit^{pos} cells were grown in DMEM/F12 modified medium for 1-3 weeks when clones were identified and expanded. The clonogenicity of the ckit^{pos} cells was determined by counting the number of clones generated in each 96-well plate and expressed as a percentage. A total of 10 plates per cardiac region were analyzed. Clonogenic cells and cardiospheres were transferred to a specific cardiogenic differentiation medium (modified from 42) for myocyte, vascular smooth muscle and endothelial cell specification.

The cell migration assay was carried out using a modified Boyden chamber, according manufacturer's instructions (Chemicon). 200 ng/ml HGF or 200 ng/ml IGF-I were placed in the lower chamber of a 24 well plate for 24 hours. For proliferation assay, 2.5×10⁴ pCSCs were plated in 24×35 mm dishes and were serum starved for 36 hrs in 0% serum DMEM/F 12 base medium. 6 dishes acted as baseline control and were supplemented with BrdU (1 μg/ml) before being fixed and stained 1 hour later. Then DMEM/F 12 base medium supplemented with 3% FBS and 200 ng/ml HGF (n=6 dishes) or 200 ng/ml IGF-I (n=6 dishes) was added to the remaining 12 dishes. 6 dishes acted as controls, with no growth factors added to the medium. BrdU was added, 1 μg/ml every 6 hours. Cells were fixed after 24 hours and BrdU incorporation was assessed using the BrdU detection system kit (Roche). The nuclei were counterstained with the DNA binding dye, 4,6-diamidino-2-phenylindole (DAPI, Sigma) at 1 μg/ml. Cells were evaluated using fluorescence microscopy (Nikon E1000M). 10 random fields at ×20 magnification were counted for each dish, and numbers expressed as a percentage of BrdU positive cells relative to the total number of cells counted.

### Immunocytochemistry

Cells were cultured on glass chamber slides (BD Falcon) for 2 days, fixed with 4% PFA for 20 min, and then stained. For intracellular staining, cells were permeabilized using 0.1% Triton X-100. Cells were incubated with the primary antibody overnight at 4° C., washed three times and then incubated with a FITC- or Texas Red-conjugated secondary antibody for 1 hr at 37° C. Then cells were washed three times, and nuclei were counterstained with DAPI. Fluorescence was visualized and images acquired with confocal microscopy (Zeiss LSM510). The following antibodies were used for cell staining: Oct3/4, Nanog, Isl-1, c-kit, FLK-1, and Nkx2.5 (R&D Systems); Bmi-1, c-met and IGF-Ir (Santa Cruz Biotechnology), telomerase (Abeam). Cardiospheres were stained for c-kit after 24 hours of culture in a glass chamber slide. After 4-6 days in culture to allow outgrowth and differentiation of cells from sphere, they were stained with antibodies against smooth muscle actin, α-sarcomeric actin (Sigma) and von Willebrand factor (DAKO). All secondary antibodies were purchased from Jackson Immunoresearch.

### Western Blot Analysis

Immunoblots to detect the IGF-I (IGF-IR) and HGF (c-met) receptors were carried out as previously described (Ellison et al. 2007. J. Biol. Chem. 282: 11397) using protein lysates obtained from c-kit^{pos} pCSCs subjected to serum starvation medium for 24 hours followed by supplementation with 200 ng/ml IGF-1 or 200 ng/ml HGF for 10-20 minutes. The following antibodies were used at dilutions suggested by the manufacturers: rabbit polyclonal Abs IGF-IR, phosphor-IGFIR, Akt, phosphor-Akt,c-met (Cell Signalling), phosphor-c-met (Abeam), FAK, and phosphor-FAK (Upstate).

### Histology

After atrial excision hearts were divided into 5 coronal slices from apex to base with cuts perpendicular to the long axis. Samples of infarcted, peri-infarcted and distal myocardium were obtained from each level from each pig. Samples were washed with PBS, fixed in 10% formalin and paraffin embedded. 5 μm sections were prepared on a microtome (Sakura) and mounted on microscope slides. Sections were stained with hematoxylin and eosin (H&E), according to standard procedures (Ellison et al. 2007. J. Biol. Chem. 282: 11397). Myocyte diameter was measured across the nucleus in H&E sections (3 slides per animal) of the peri-infarct region from levels C and D, on a light microscope (Nikon E1000M) using Lucia G software. A total of 200 myocytes per section were analyzed for each pig.

To determine myocardial fibrosis, sections of the infracted myocardium were stained with Sirius red as previously described (Lee, C G. et al., 2001. J. Exp. Med. 194:809). Serial sections were fixed in 10% formalin in PBS for 20 min After washing in distilled water for 5 min, sections were incubated at room temperature for 30 min in 0.1% Fast Blue RR in Magnesium Borate buffer at pH 9 (Sigma). Then sections were washed in distilled water before incubation at room temperature for 10 min in 0.1% Sirius red in saturated picric acid (Sigma). Sections were further washed in distilled water before they were dehydrated, cleared and mounted. In this protocol, connective tissue (mainly collagen) stains red and muscle stains yellow/orange. Semi-quantitative evaluation of the amount of myocardial connective tissue was carried out using Lucia G image analysis at ×40 magnification. Percent collagen (percent area of positive staining) was determined in the entire infarct zone. A total of 3 slides were assessed per animal for each level, and an average obtained.

### Immunohisochemistry and Confocal Microscopy

To identify CSCs, transverse pig heart sections were stained with antibodies against the stem cell antigen, c-kit (rabbit polyclonal, Dako). c-kit^{pos} CSCs were identified as lineage-negative (Lin^{neg}), by staining negative for markers of haematopoietic, neural, and skeletal muscle lineages (21). For quantification of CSC myocardial distribution in the different cardiac regions of control pigs, the number of c-kit^{pos} (lin^{neg}) cells and cardiomyocytes was counted for a total of 5 sections at ×63 magnification. The area of each cross section was then measured, and the number of CSCs and cardiomyocytes per unit area was determined. The data for the atria were pooled, due to few differences found between the number of c-kit^{pos} CSCs in the left and right atria. The number of CSCs was expressed per 10⁶ myocytes.

Cycling cells were identified by BrdU (Roche) and Ki67 (Vector labs) staining. Progenitor cells stained positive for c-kit and the transcription factors, Nkx2.5 (R&D Systems), Ets-1 and GATA6 (Santa Cruz Biotechnology). Newly formed myocytes were identified with antibodies against BrdU, Ki67 and α-sarcomeric actin (Sigma), cardiac troponin I (Santa Cruz Biotechnology) or slow (cardiac) myosin heavy chain (Sigma). Newly formed vascular structures were detected by staining for BrdU and α-smooth muscle actin (mouse monoclonal, Sigma) or vWF (rabbit polyconal, Dako). Images were acquired using confocal microscopy (Zeiss 510 LSM). The number of CSCs, myocyte progenitor cells (c-kit^{pos}/Nkx2.5^{pos}), and newly formed myocytes (BrdUP^{pos} and ki67^{pos}) were quantified for the infarct, peri-infarct and distal regions in each level. A total of 3000 cells (−20 fields) were counted for each region at ×63 magnification. 3 slides per animal were assessed. Numbers were expressed as a percentage relative to the total number of cells counted. The size of 50 BrdUP^{pos} newly formed myocytes per animal in the infarct and peri-infarct regions was measured using Lucia G software.

The density of capillaries in the infarct region was evaluated by staining with an antibody against vWF (DAKO). The 2⁰ Ab used was a donkey anti-rabbit, conjugated with HRP (Santa Cruz). Endogenous peroxidase in the section was blocked with 3% hydrogen peroxide in PBS for 15 minutes at room temperature. The chromogen 3,3-diaminobenzidine (DAB) (Sigma) was used to visualize the blood vessels. The slides were counterstained with hematoxylin for identification of nuclei. The number of capillaries (defined as 1 or 2 endothelial cells spanning the vWF-positive vessel circumference) was determined by counting 10 fields/section in the infarct zone in levels C and D at ×40 magnification. A total of 3 slides/animal were assessed. The number of capillaries was expressed per 0.2 mm²

To detect cellular apoptosis, sections were stained with rabbit anti-human activated caspase-3 primary antibody (R&D Systems) and a donkey anti-rabbit HRP-conjugated 2° Ab. The chromogen DAB (Sigma) were used to visualise the apoptotic cardiomyocytes. Sections were then counterstained with haematoxylin and permanently mounted before being examined by light microscopy. The number of caspase-3 positive myocytes in the peri-infarct zone of levels C and D was determining by counting 20 random fields/section at ×40 magnification. A total of 3 slides/animal were assessed. The amount of caspase-3 positive myocytes was expressed as percentage relative to the total number of myocytes counted.

### Statistical Analysis

Data are reported as Mean±SD. Significance between 2 groups was determined by Student's t test and in multiple comparisons by the analysis of variance (ANOVA). Bonferroni post hoc method was used to locate the differences. Significance was set at P<0.05.

Acute MI was induced in Dallas landrace pigs (68±4 kg, n=18) by a 75-min coronary balloon occlusion of the left circumflex artery. After 1 month, all survived animals underwent intramyocardial injections (10 injections of 0.2 mL each) with the NOGA delivery system of IGF-1/HGF dissolved in saline (both 0.5 μg/ml; n=5, GF), or IGF-1/HGF incorporated in UPy-hydrogel (both 0.5 μg/ml, n=5, UPy-GF). UPy-hydrogel without added growth factors was administered to 4 control (CTRL) pigs, which have undergone the same MI protocol as the test animals. The pigs were sacrificed for functional endpoint analysis and immunohistological analysis 1 month after the GF administration.

### Example 1

### Preparation of PLGA Microspheres

Two sets of microspheres of PLGA and alginate were prepared; one set containing a mixture of human serum albumin (HSA) and insulin-like growth factor 1 (IGF-I), the other set containing a mixture of HSA and hepatocyte growth factor (HGF). The HSA was used to provide enough bulk for the emulsion given the very small quantities of the growth factors needed.

The conditions used to form the PLGA microspheres are the following: A nebulizer Flow Focussing of Ingeniatrics (D=150 nm, H=125) was employed in a configuration liquid-liquid in which the focused liquid is the emulsion of PLGA+HSA+growth factor and the focusing liquid is water.

The lipid phase consisted of: 5% PLGA in EtOAc (ethyl acetate)

The aqueous phase consisted of: 5% HSA, 0.1% growth factor, 0.45% NaCL, 0.25% Tween 20 in H2O.

The mixture of the two phases was sonicated for 30 min

The microdroplets are produced in a bath of 2% polyvinyl alcohol (PVA, Fluka Chemica).

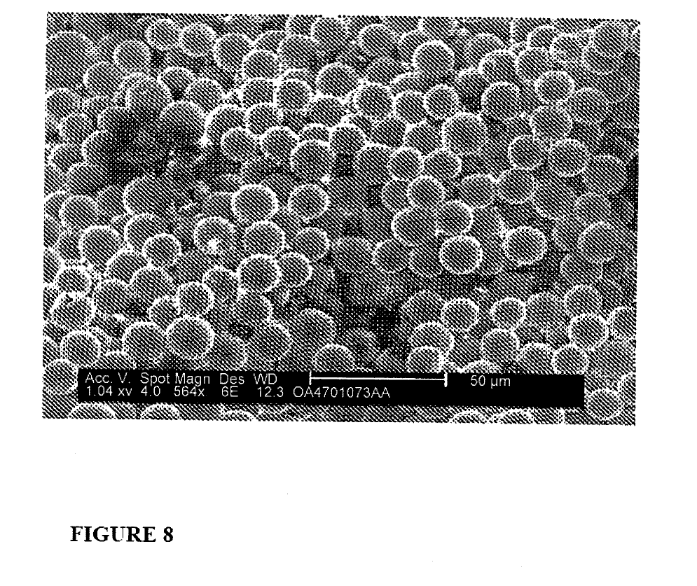

The size of the particles is controlled by the flow volume of the focused (Qd) and focusing (Qt) fluids. To obtain particles of 15±1 microns, a Q_{d}=3.5 mL/h and a Qₜ=3 mL/h were used. The efficiency of encapsulation of HSA+IGF-1 mixture was of 37%. The size of the particles was ascertained by optical and electron microscopy (see FIG. 8).

The same procedures with minor modification were used to prepare HGF— containing PLGA particles.

### Example 2

### Optimization the Production of Monodisperse PLGA Microspheres of 15 μm Diameter

To optimize the efficiency of encapsulation in order to reduce the number of microspheres to be administered the conditions used were optimized with modification in the following parameters:

a.—Incorporation of emulsifiers in the lipid phase. The optimal combination was found to be a mixture of Tween 80 and Span 60 which produced emulsion stable for up to 5 hours

b.—Optimization of the concentration of protein (Human Serum Albumin), HSA of 20% instead of 5%.

c—Optimization of the concentration of NaCl in the aqueous phase to 0.2% instead of 0.45%.

d.—Optimization of the PLGA concentration to 5.5% instead of 5% in EtOAc.

e.—The concentration of HGF-I in the initial mix was 0.4%

Therefore the aqueous phase consisted of 20% HSA, 0.4% IGF-I; 0.2 NaCl; 0.1 Tween 20; 0.15 Span 60. The organic phase consisted of 5.5% PLGA in EtOAc (ethyl acetate).

The microparticles were obtained by simple flow focusing the conditions described in Example #1. The size of the particles, as determined by SEM was of 14.36 μm with a SD of 0.91 and an efficiency of encapsulation of 82.4 with an entrapment of 13.1%. Protein determinations complemented by quantitative ELISAs documented that each 1×10⁶ microspheres carried 3 μg of IGF-1 and 348 μg of HSA. Biological in vitro assays of the IGF-I contained in the microspheres tested by their capacity to bind and activate the IGF-1 receptor of live cells show that after one round of liophylization and resuspension the encapsulated IGF-I maintained 82% of the original biological activity. Therefore, each one million of microspheres had a biological activity equivalent of 2.5 μg of the native IGF-I.

Similar protocols were used to encapsulate HGF, with a final result of 1.7 μg HGF encapsulated per 1×10⁶ particles with a biological activity of 63% of the original. Thus, each million of HGF microspheres can deliver the equivalent of 1 μg of active HGF.

The encapsulation of SCF (Stem Cell Factor), the ligand for the c-kit receptor, produced particles containing 2.3 μg SCF per 1×10⁶ microspheres with an activity 76% of the original solution as determined through activation of the c-kit receptor.

### Conclusion:

The single flow focusing procedure used is very efficient in the encapsulation of a mixture of HSA and different growth factors. Changing the initial ratio of HSA to growth factor it is possible to reach loading values of up to 350 μg of the desired pharmacological protein per 1×10⁶ microspheres of PLGA of 15 μm of diameter with a variation coefficient of ≦6%.

### Example 3

Production of monodisperse ALGINATE microspheres and encapsulation of IGF-I The reagents and equipment used for the production of the microspheres were the following:

Alginate: Protanal LF 10/60; FMCBioPolymer (G/M≧1.5); Protanal LF10/60LS; FMCBioPolymer (G/M≦1).

HSA (human serum albumin, 97-99%, A9511) from Sigma-Aldrich—IGF-1 from PreProtect

CaCl₂; tribasic sodium citrate

Nebulizers FF simple in the configuration liquid-gas: L2 (D=100 μm, H=100) and L3 (D=100 μm, H=100).

Harvard pump 11 plus.

After more than 120 assays to establish the appropriate conditions, it became evident that a mixture of alginates gave better results than a single alginate. Protanal LF 10/60: Protanal LF10/60LS at a ratio 0.7%:0.3% gave the optimal results. The optimal distance for nebulization was found to be 10 cm. The optimal concentration of HAS in the mix was 14% and IGF-I 0.3%. This mixture is nebulized using the FF (D=100 μm, H=100) in configuration liquid-gas (ΔPt=300 mbar, Q_{d}=5 mL/h using gas as the focusing fluid. The nebulizer is placed at 10 cm of a solution of 3% CaCl₂ in a shaking bath, collected by centrifugation after 30 min and washed to remove the CaCl₂. The size distribution of the particles is determined by flow cytometry and SEM. The efficiency of encapsulation of HSA by protein quantification and standard curves. The encapsulation of hrHGF-1 was determined by ELISA as described in Example #2.

The size of the particles, as determined by SEM was of 15.87 μm with a SD of 1.83 and an efficiency of encapsulation of 71.4 with an entrapment of 11.6%. Protein determinations complemented by quantitative ELISAs documented that each 1×10⁶ microspheres carried ˜2 μg of IGF-1 and 269 μg of HSA. Biological in vitro assays of the IGF-I contained in the microspheres tested by their capacity to bind and activate the IGF-1 receptor of live cells show that after one round of liophylization and resuspension the encapsulated IGF-I maintained 67% of the original biological activity. Therefore, each one million of microspheres had a biological activity equivalent of ˜1.5 μg of the native IGF-I.

This protocol can be adapted to be used with different types of polymers such as Poly ether-polyester segmented block copolymers of polybutylene terephthalate (PBT) and polyethylene oxide (PEO) Poly Active^{R} using the FF nebulizer as well as other spraying methods.

### Conclusion:

Alginate is an adequate polymer for the production of monodisperse microspheres with an approximate diameter of 15 μm and to encapsulate large amounts of proteins. The protocols used can be modified to increase the ratio of IGF-I to HSA up to 60:40 which increases the load of active compound by more than two orders of magnitude. From the results obtained, the range of sizes around the peak of 15 μm is narrower when using PLGA than with the combination of alginates tested here. Given the large number of different alginate preparations it is likely that the homogeneity of the microparticles found here could be significantly improved.

### Example 4

To produce microspheres where the active compound is located on the surface of the particle it is possible to produce the microspheres shown above using a polyelectrolyte instead of PLGA of charge of opposite sign to the active to be bound. Examples of such polyelectrolytes are gum Arabic, pectins, proteins, nucleic acids, polysaccharides, hyaluronic acid, heparin, carboxymethylcellulose, chitosan, alginic acid and a multitude of synthetic polymers. When the polyelectrolyte has a charge of opposite sign to the active compound, it is possible to attach it to the microparticle by absorption from a solution of the active.

### Example 5

Microspheres of 15 μg in diameter are optimal for capillary entrapment after intracoronary administration without spillover to the systemic circulation.

Female Yorkshire white pigs (n=2) (27 kg) were sedated with telazol (100 mg, I.M.), intubated and shaved. An intravenous catheter was placed in a peripheral ear vein. The animals were moved to the surgery room, placed onto a support board, and secured to the surgical table with limb bindings Animals were maintained anesthesized with isoflurane (2.5% in 0₂) and their EKG monitored continuously throughout the procedure. Using a portable radiological source (GE STENOSCOP, GE Medical Systems USA) for fluoroscopic guidance, the left main coronary artery was intubated with a 6F guiding catheter JR 3.5 of 40 cm in length specially designed for the protocol (Cordynamic-Iberhospitex S. A. Barcelona, Spain). A baseline coronary angiography was performed.

In both animals, a coronary guide catheter of 2 mm diameter was advanced over a guide wire (Hi-Torque Balance Middle-Weight 0.014″) to the origin of the left coronary artery. Through this catheter was advanced a microcatheter of 0.014″ (0.3 mm) internal diameter and its tip positioned in the proximal portion of the left anterior coronary artery (LAD), just below the origin of the first perforating artery. This is the same location used to produce the experimental myocardial infarction and for the administration of the solution of growth factors described above. Another catheter was placed into the coronary sinus to collect cardiac venous blood samples during the procedure. Before starting the administration a peripheral, coronary venous and arterial blood sample was collected. In the case of abundant ventricular extra-systoles or ventricular fibrillation, Lidocaine of 1-3 mg/kg was administered intravenously. Pre-operative medication was administered as 75 mg clopidrogel (Plavix) and 250 mg aspirin one day before surgical procedure. Postoperative medication consisted of 75 mg clopidrogel (Plavix) and 125 mg aspirin daily until the sacrifice.

To determine the optimal size of the microspheres to be fully trapped in the capillary network a mixture of fluorescent polystyrene microspheres of diameters 2 μm, 4 μm, 6 μm, 10 μm; 12 μm and 15 μm, each labeled with a different dye (purchased from Invitrogen and from Polysciences Inc., Cat #F8830, F8858; F8824; Polybead Black dyed microsphere 6.0 μm, Megabead NIST 12.0 μm and F8842) were in mixed in a suspension of 20 mL of PBS at a concentration of 1×10⁶ microspheres of each of the 6 sizes per mL and vortexed for 5 min to insure an homogeneous suspension. This suspension was administered at the origin of the left coronary artery of three pigs through the angiography catheter by a Harvard pump at a rate of 1 mL/min After administration of each mL (1 million microspheres) the injection was suspended for 3 min during which time a coronary sinus blood sample was taken. Immediately after obtaining the blood samples, blood smear slides were prepared to check for the presence of fluorescent microparticles. After the complete administration of the 20 mL microsphere suspension coronary sinus blood samples were collected for an additional 3 hours at every 30 min intervals. At the conclusion of the experiment the animals were sacrificed and the heart excised, fixed and samples were taken for sectioning followed by histological and fluorescent microscopy examination.

Because the microspheres of different sizes were administered in equal numbers their ratios in the coronary sinus venous flow and in the myocardium should be mirror images of each other. Those particles that go through the capillary bed should have a high concentration in the coronary sinus blood and low in the myocardium at the end of the experiment. The reverse should be true for the particles that do not pass through the capillary bed. As shown below, only sizes <10 μm are eff[iota]cienly retained in the myocardium but even microspheres of 10 and 12 μm leak through to a meaningful extent since between 19 and 8%, respectively of these microspheres passed into the systemic circulation. On the other hand, >1% of the 15 μm particles passed through the capillary bed and reached to coronary sinus.

TABLE 2

| Microsphere size in μm | 2 | 4 | 6 | 10 | 12 | 15 |
|---|---|---|---|---|---|---|
| Outflow into coronary | 95 | 73 | 53 | 19 | 8 | ≧1 |
| Sinus (calculated in % | | | | | | |
| Retained in the myocardium | ≦3 | 15 | 41 | 77 | 90 | 99 |
| 3 h after administration in % | | | | | | |

To determine whether the results shown above were specific for the myocardium or could be extended to other tissues, the same protocol was used to administer an identical suspension of microspheres through the femoral artery of the right leg. Blood samples were collected from the femoral vein and quadriceps muscle samples were analyzed to determine the permanence of the different microspheres in the skeletal muscle. The results are summarized in Table #3.

TABLE 3

| Microsphere size in μm: | 2 | 4 | 6 | 10 | 12 | 15 |
|---|---|---|---|---|---|---|
| Outflow into the venous | 92 | 67 | 59 | 12 | 11 | ≧1 |
| return (calculated) in % | | | | | | |
| Retained in the skeletal | ≦1 | 11 | 27 | 72 | 83 | ≧99 |
| Muscle 3 h post in % | | | | | | |

Conclusion:

The minimum size of microspheres that insures >99% retention in the tissue of interest is 15 μm in diameter. Because it is important to use the minimum effective size in order to minimize the production of micro foci of ischemia by obstructing precapillary arterioles, this diameter size is the optimal for the local delivery of substances to a particular tissue through its capillary bed.

### Example 6

### Administration of the Microspheres in the Coronary Circulation

A 20 mL suspension of fluorescent polystyrene microspheres of 15 μm (Invitrogen, Cat # F8842, FluoSpheres(R) polystyrene microspheres) at a concentration of 1×10⁶/mL in PBS was prepared and vortexed for 5 min This suspension was administered through the angiography catheter by a Harvard pump at a rate of 1 mL/min at the origin of the main left coronary artery. After administration of each mL (1 million microspheres) the injection was suspended for 3 min during which time a complete EKG and a coronary sinus blood sample was taken. Immediately after obtaining the blood samples, blood smear slides were prepared to check for the presence of fluorescent microparticles. The rest of the sample was saved for enzyme determinations. The procedure was continued until the electrocardiogram showed minimal alterations consistent with myocardial ischemia. Coronary blood flow (TIMI) was measured at the start of the experiment and after the administration of the particle suspension. The two pigs were allowed to recover, re-examined at 24 hours and sacrificed thereafter.

### Results:

In animal #1 the first EKG alterations were detected after the administration of 16 mL of the suspension (16 million microspheres). In the second animal EKG alterations did not appear until after the administration of 18 mL (18 million microspheres). In both animals, the coronary blood flow was TIMI 3 (normal) at the end of the procedure. Animal #1 was sacrificed 24 hours after termination of the infusion.

A complete EKG and blood samples were collected before sacrifice. The heart was processed for macroscopic and microscopic examination.

Animal #2 at 24 hours had a normal EKG and coronary blood flow (TIMI 3). After obtaining a set of blood samples the animal was sacrificed and the heart processed for macroscopic and microscopic examination.

All the blood smears from the samples taken from the coronary sinus and from the systemic circulation from animals #1 and #2 were examined by fluorescent microscopy at low and high magnification. No fluorescent beads were detected in any of the samples. This indicates that trapping in the capillary network of microspheres 15 μm in diameter is very efficient. Moreover, if there are any functional shunts from the coronary arteries to the right ventricle with this method of injection through the Thebesius veins, they are minor and not detected by the methods employed here.

The enzyme measurements (Table 4) show that animal #1 developed a small myocardial infarction as shown by the increased level of cardiac specific troponin T (TnT) in blood (values higher than 0.01 ng/ml are abnormal), while the values of animal #2 are normal and suggest that this animal developed only transient ischemia during the administration of the particles and recovered without any permanent myocardial damage. This interpretation was confirmed by the pathology as shown below. The macroscopic section of the heart of animal #1 shows micro foci of necrosis (pale areas) while the section of animal #2 is normal. This conclusion was confirmed by the histopathology (data not shown).

TABLE 4

| | | PRE | PRE | POST | | POST | POST |
|---|---|---|---|---|---|---|---|
| | Marker | INJ CS | INJ | CS | POST | 14 H | 24 H |
| PIG1 | CK | 574 | 669 | 423 | 567 | 1920 | 1982 |
| | MB | 521 | 646 | 506 | 498 | 919 | 1231 |
| | TrT | 0.01 | 0.01 | 0.01 | 0.01 | 1.72 | 1.35 |
| PIG2 | CK | 1120 | 1114 | 1099 | 1073 | 1834 | 1895 |
| | MB | 922 | 791 | 920 | 523 | 867 | 739 |
| | TrT | 0.02 | 0.01 | 0.04 | 0.01 | 0.01 | 0.01 |

Conclusion:

Administration of up to 15×10 microspheres 15 μm in diameter in the area irrigated by the left anterior descending artery (LAD) in a heart is well tolerated and does not produce myocardial damage. Doses above 15×10 microspheres have a high risk of producing small ischemic areas that might leave permanent scar. Therefore, with a loading in the mid-range of the values obtained with the PLGA as the polymer of 1 mg of protein per 1×10⁶ microspheres of 15 μm diameter, it is possible to deliver up to 15 mg of the therapeutic agent to the capillary bed of the myocardium irrigated by the left coronary artery.

### Example 7

### Administration of PLGA Microbeads Loaded with Growth Factors

Once the safety dose range of 15 μm microspheres has bee determined, the same protocol was used to administer 10×10⁶ PLGA microspheres (15 μm in diameter) to the same region of the myocardium. The microsphere suspension was composed of 4×10 PLGA microspheres loaded with a total of 2 μg of human recombinant insulin-like growth factor 1 (IGF-I); 4×10⁶ PLGA microspheres loaded with a total of 1 μg of human recombinant hepatocyte growth factor (HGF). These two types of microspheres were also loaded with a fluorescent green dye in order to make easier their visualization in the blood and in the histological sections. In addition, the suspension contained 2×10⁶ polystyrene fluorescent in the orange range from Invitrogen. The Invitrogen spheres were included to serve as control for the stability and distribution of the PLGA microspheres. The suspension in 10 mL of physiological PBS, was administered to the instrumented pigs as described above.

The administration of the suspension to the two animals was uneventful and there were no electrocardiographic signs of ischemia. The capillary blood flow was normal during and after the procedure (TIMI 3). One animal (pig #3) was sacrificed 30 min after the procedure and the other (pig #4) at 24 hours after the procedure. Both hearts were processed for macroscopic and microscopic analyses.

Neither the peripheral nor the coronary sinus blood samples of these two animals showed the presence to either Invitrogen or PLGA beads in the multiple blood smears. Preliminary analysis of lung, liver and spleen sections of these two animals also failed to detect the presence of either type of microspheres.

TABLE 5

| | | PRE | PRE | POST | | POST | POST |
|---|---|---|---|---|---|---|---|
| | Marker | INJ CS | INJ | CS | POST | 14 H | 24 H |
| PIG3 | CK | 589 | 692 | 432 | 657 | | |
| | MB | 527 | 626 | 560 | 418 | | |
| | TrT | 0.01 | 0.01 | 0.01 | 0.01 | | |
| PIG4 | CK | 467 | 468 | 441 | 442 | | 434 |
| | MB | 451 | 505 | 562 | 378 | | 411 |
| | TrT | 0.01 | 0.01 | 0.01 | 0.01 | | 0.01 |

Legend for Tables 4 and 5.

Markers: CK, creatine kinase; MB, the MB isoform of creatine kinase which is cardiac specific; TrT, Cardiac troponin T, which is the most specific and sensitive marker for myocardial damage. PRE INJ CS, blood sample taken from the coronary sinus at the start of the procedure; PRE INJ, systemic blood sample taken at the start of the procedure; POST CS, blood sample taken from the coronary sinus at the end of the procedure; POST, blood sample from systemic circulation taken at the end of the procedure; POST 14H, systemic blood sample taken at 14 hours after the procedure; POST 24H, systemic blood sample taken 24 hours after the procedure before sacrificing the animal.

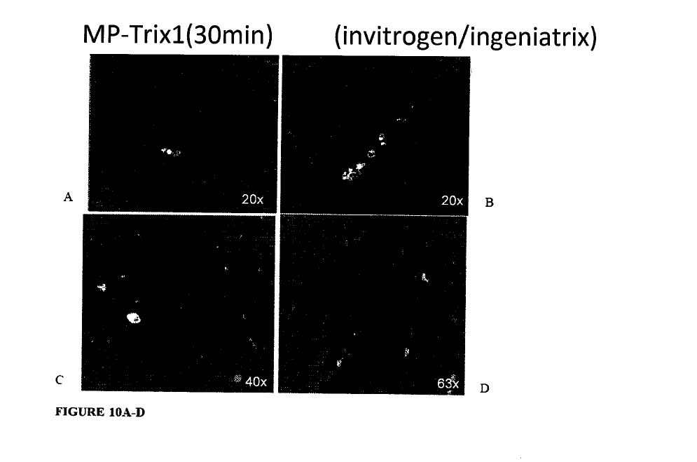

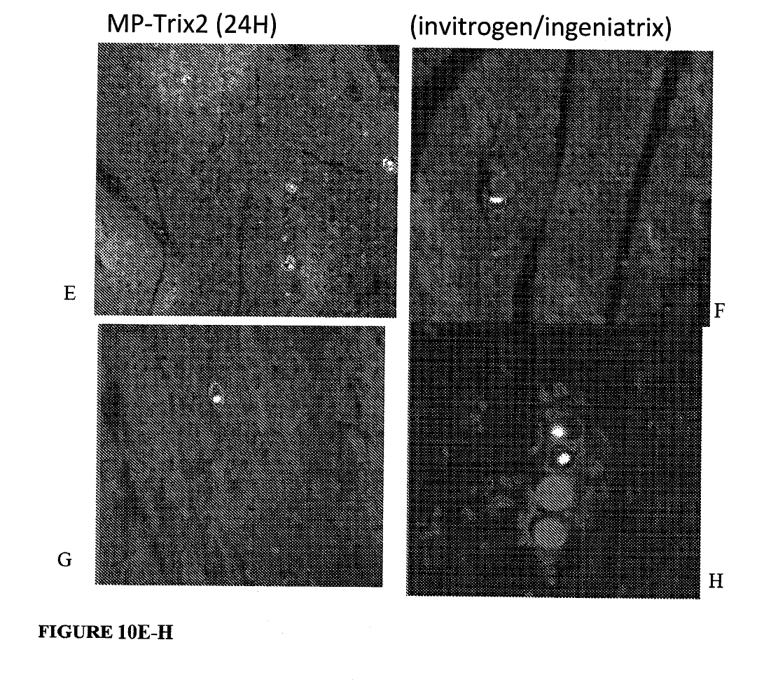

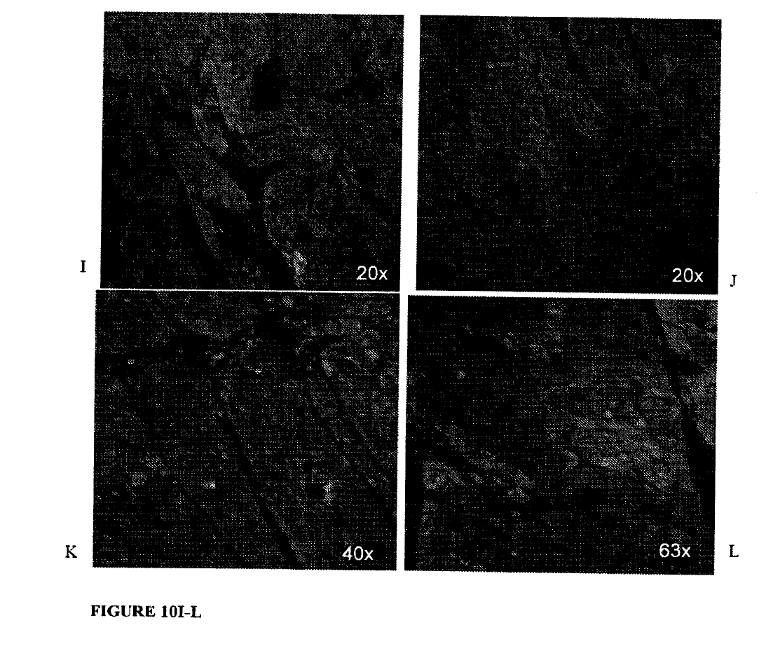

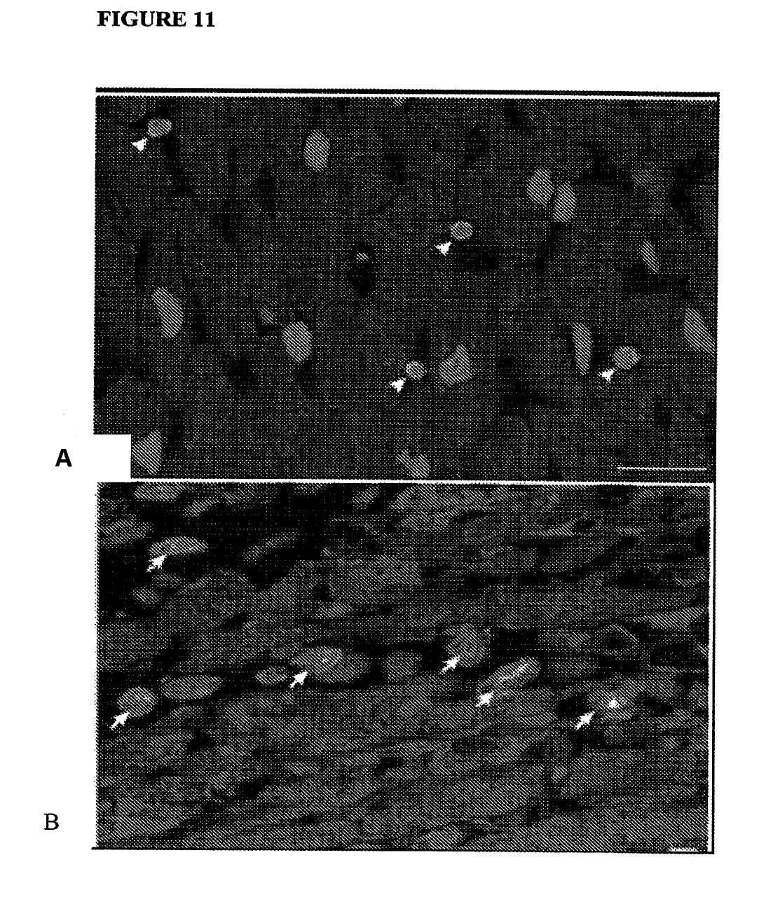

The macroscopic sections of these two animals were completely normal (not shown). The analysis of the section of pig #3 under the fluorescent microscope showed the distribution of the PLGA beads (green) and the polystyrene beads (red/orange) in the capillary vessels in the approximate ratio of 1:4 (FIG. 10 below), as would be expected from the composition of the mixture administered. There was no evidence of any microscopic tissue damage in any of the regions of the heart examined. In pig #4 the number of PLGA beads (green) has already decreased significantly and the ratio of these beads to the polystyrene ones (red/orange) is closer to 1:1 (see FIG. 11), indicating that the PLGA beads become degraded with a half life of ˜16 hours.

Effectiveness of IGF-I and HGF administered in microspheres to stimulate the resident cardiac stem cells.

As described above, the combination of IGF-I and HGF administered through the coronary arteries was very effective in stimulating the activation of the resident cardiac stem cells. In this preliminary assay we monitored the activation of the stem cells in the region were the microspheres were delivered and compared it to a region of the left ventricle not irrigated by the left coronary artery. As can be seen in the images in FIG. 11, most resident stem cells in the non-treated myocardium are quiescent (highlighted by arrows/arrow heads) while those of the treated region have entered into the cell cycle, as demonstrated by the expression of the cell cycle marker ki-67 (yellow signal in the nucleus—in Figures the light “spots” in the highlight areas). Therefore, administration of growth factors on a solid substrate that delivers them to the capillaries and keep them there until they have unloaded into the surrounding interstitial space, is an effective method of growth factor administration for the stimulation of the endogenous stem cell population.

Conclusion:

Local delivery of IGF-I and HGF to particular regions of the myocardium by mean of biodegradable microbeads of a diameter which does not allow they to cross the capillaries and enter the systemic circulation is effective in stimulation the resident stem cells of particular regions of the tissue without affecting those not targeted by the therapy.

### Example 8

### Porcine c-kit^{pos} Cardiac Stem and Progenitor Cells are Multipotent and Phenotypically Similar to Those of Other Animal Species

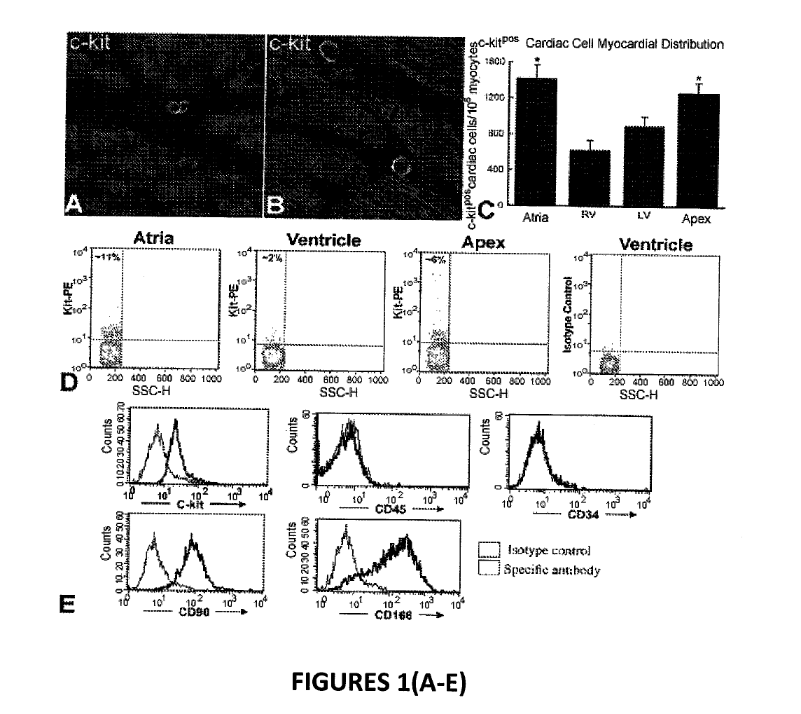

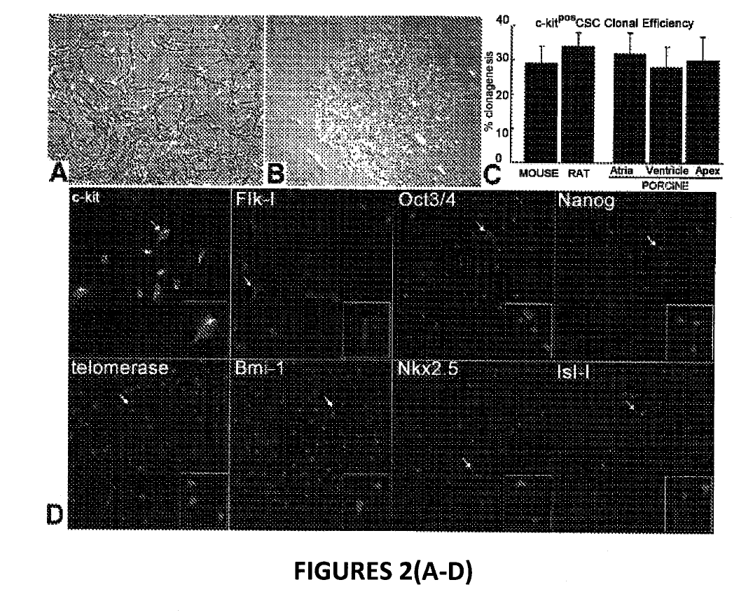

Histological sections of myocardium from 3 Yorkshire pigs weighing 24±3 kg were examined by confocal microscopy for the presence of cells positive for the common stem cell marker, c-kit, the receptor for stem cell factor (SCF), known to be expressed by the majority of CSCs. Small cells positive for c-kit (c-kit^{pos}) were distributed throughout the atrial and ventricular myocardium (FIG. 1A-B) with a higher density in the atria (no difference between left and right atria, data not shown) and the ventricular apex, compared to other cardiac regions (FIG. 1C). This distribution pattern matches the anatomical location of the c-kit^{pos} CSCs in the hearts of other animal species, including humans. Accordingly, the density of c-kit^{pos} cells in the pig heart is similar to human and rodent myocardium: 1 cell per 1,000 myocytes or −50,000 c-kit^{pos} cells per gram of tissue.

Myocardial tissue samples from different porcine cardiac regions were enzymatically digested to obtain a myocyte-depleted cell population. c-kit^{pos} cells constituted 10±3%, 3±2% and 7±% of the starting myocyte-depleted cardiac cell population from the atria, ventricle, and apex, respectively (FIG. 1D).

The c-kit^{pos} cells were separated using MACS technology (21) which yielded a highly enriched cell preparation constituted by >90% of c-kit^{pos} cells (FIG. 1E). FACS analysis showed that the c-kit^{pos} enriched cardiac cells were negative for the pan leukocyte marker CD45 and the endothelial/hematopoietic progenitor marker CD34 (FIG. 1E). A high fraction (87%) of c-kit^{pos} porcine cardiac cells expressed CD90, (a common non-specific mesenchymal marker) and CD 166 (adhesion molecule) (FIG. 1E). Only a small fraction was positive for the markers of hematopoietic/endothelial progenitors, CD 105 and C D 133 (Suppl FIG. 1). c-kit^{pos} cardiac cells were negative when analyzed for a panel of CD markers specific for other hematopoietic, mesenchymal and endothelial cell lineages, including CD13, CD14, CD31, CD38, CD44, CD33. From these analyses we can conclude that the porcine c-kit-sorted cardiac cells are c-kit^{pos}, CD90^{pos}, CD166^{pos}, CD105^{low}, CD133^{low} and CD45^{neg}, CD34^{neg}, CD3^{neg}, CD44^{neg}.

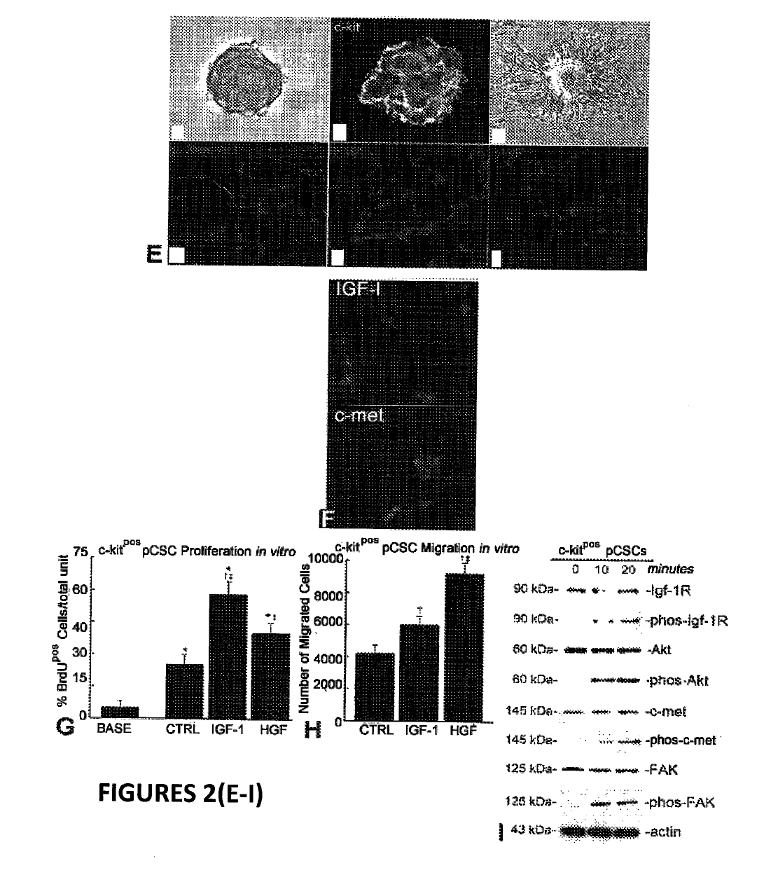

Freshly isolated c-kit^{pos} cardiac cells from atria, ventricles and apex were expanded in culture (4 passages) and then deposited as a single cells into 96-well Terasaki plates to generate single cell clones (FIG. 2A-B). The clonal efficiency of the porcine cells was similar for all cardiac locations and to the previously reported cloning efficiency of the rodent CSCs (FIG. 2C) (Beltrami et al. Cell 2003). We randomly picked 2 clones each from atria, ventricle and apex-derived cells and further expanded them. These clones showed a ˜30 hours doubling time and have been propagated so far for more than 65 passages and serially sub-cloned every 10 passages, without reaching growth arrest or senescence. These c-kit^{pos} cardiac cell clones have maintained a normal karyotype without detectable chromosomal alterations.

Cloned c-kit^{pos} porcine cardiac cells were analyzed for markers of sternness and cardiac-lineage commitment using immunocytochemistry. Cells showed positivity for c-kit (90±8%), FIk-I (86±9%), Oct3/4 (62±11%), Nanog (46±5%), telomerase (81±10%), Bmi-1 (70±14%), Nkx2.5 (52±8%), IsI-I (8±6%) (FIG. 2Di. Because the clones originated from single cells, the wide expression of the multipotency genes in their progeny suggested that the level of expression of these genes in the parental cell population is very high. Unfortunately, the primary population of c-kit^{pos} cells is a mixture of CSCs, progenitors and precursors and we do yet have markers specific for the ‘real’ CSCs. Therefore, it is only possible to infer the phenotype of these cells through the analysis of their descendants.

When cloned c-kit^{pos} porcine cardiac cells were plated in modified cardiosphere formation medium (mCSFM) in bacteriological dishes (Corning), they grew in suspension and generated spherical clones, named cardiospheres (FIG. 2E) (Beltrami, A. P. et al., 2003. Cell 114:763; Oh H, Bradfute S B, Gallardo T D et al. Cardiac progenitor cells from adult myocardium: homing, differentiation, and fusion after infarction. Proc Natl Acad Sci USA 2003; 100(21): 12313-12318; Matsuura K, Nagai T, Nishigaki N et al. Adult cardiac Sea-1-positive cells differentiate into beating cardiomyocytes. J Biol Chem 2004; 279(12):11384-11391). When cardiospheres were placed in laminin-coated plastic dishes with cardiogenic differentiation medium, they attached and cells spread out from the sphere acquiring a flat morphology (FIG. 2E). Four to six days after plating, these peripheral flat cells expressed proteins specific for myocyte (27±4%), endothelial (10±6%) and smooth muscle cell (34±5%) lineages (FIG. 2E). These results show that porcine c-kit^{pos} cardiac cells have true stem cell characteristics, i.e. they express markers of sternness, are clonogenic, self-renewing, and multipotent. Thus, porcine c-kit^{pos} cardiac stem cells (hereafter identified as pCSCs) have a pattern of gene expression and a phenotype consistent with c-kit^{pos} CSCs isolated from other species (Ellison et al., 2007. J. Biol. Chem. 282: 11397).

Porcine CSCs Express Intact IGF-I, HGF and SCF Signaling Pathways that Modulate their Activation

The results show the presence of true pCSCs in the porcine heart. pCSCs express IGF-I and c-met receptors in vivo and in vitro (FIG. 2F). When grown in culture, freshly isolated pCSCs respond to the stimulation by hrIGF-1, hrHGF and hrSCF with cell proliferation (FIG. 2G) and migration (FIG. 2H). Upon ligand binding, specific downstream effector pathways were activated in pCSCs (FIG. 21). Similar results were obtained with cells from the expanded single cell clones (data not shown). Therefore, pCSCs have functionally coupled GF receptor systems that can be exploited in vivo to test myocardial regeneration protocols.

### Example 9

Production of Myocardial Infarction in Pigs, Monitoring of Ventricular Function and Myocardial Regeneration by In Situ by Stimulation of Resident Cardiac Stem Cells with Growth Factors

All animal studies were approved by proper committees of Escuela Veterinaria y Hospital de Leon, Leon, Spain. Female Yorkshire white pigs (n=26) (27±3 kg) were sedated with telazol (100 mg, I.M.), intubated and shaved. An intravenous catheter was placed in a peripheral ear vein. The animals were moved to the surgery room, placed onto a support board, and secured to the surgical table with limb bindings. Animals were maintained anesthesized with isofurane (2.5% in O2). In all 26 animals, a coronary balloon catheter was advanced over a guide wire and positioned in the proximal portion of the left anterior coronary artery (LAD), below the origin of the first perforating artery. Pigs were given 125 UI/kg of heparin before the infarction was induced and then heparin infusion (10 UI/kg/h) during the infarction procedure. To induce infarction, the LAD coronary artery was occluded by balloon inflation (2.5 mm diameter) for 75 mins. For anti-arrhythmic medication, pigs were continuously infused throughout the procedure with Amiodarona (Trangorex) (5 mg/kg/h) beginning 15 minutes before the infarction. In the case of abundant ventricular extra-systoles or ventricular fibrillation, Lidocaine of 1-3 mg/kg was administered intravenously. Pre-operative medication was administered as 75 mg clopidrogel (Plavix) and 250 mg aspirin one day before surgical procedure. Postoperative medication consisted of 75 mg clopidrogel (Plavix) and 125 mg aspirin daily until the sacrifice. Human recombinant IGF-I and HGF (Peprotech) were administered in differential doses (ranging from 2 μg to 8 μg of IGF-I and from 0.5 μg to 2 μg of HGF) to 17 pigs through a perfusion balloon catheter advanced immediately distal to the origin of the first septal artery 30 minutes after coronary reperfusion. The GFs were administered in 15 ml of PBS at a rate of 2.5 ml per minute with a 2 min reperfusion after every 5 ml administration. Saline alone was injected in another 9 pigs with MI (saline-placebo control group; CTRL) using the same protocol. Five (2 in the CTRL group and 3 in the GF groups) of the 26 animals died during acute myocardial infarction (AMI) (acute mortality of −30%). Subsequently, 3 animals died in the postoperative period: one animal on day 1 (CTRL group), one animal on day 13 (CTRL group) and one animal on day 14 (GF group). Of the remaining 18 pigs completing the study protocol, 13 were in the GF-treated groups and 5 in the CTRL group. Specifically, of the surviving 18 GF-treated animals, 4 received a I× dose of the GFs (2 μg IGF-I and 0.5 μg HGF; GF-I×), 5 animals received a 2× dose (4 μg IGF-I and 1 μg HGF; GF-2×) and 4 animals received a 4× dose (8 μm of IGF-I and 2 μm of HGF GF-4×)). Directly after the GFs or saline alone administration, all surviving animals were implanted with an osmotic pump loaded with 10 ml of a 0.5 M solution of BrdU for the duration of the study. Pigs were sacrificed at 21 days after MI and growth factor administration. The group to which each pig belonged was kept blind for investigators carrying out the immunohistochemical analysis.

Cardiac Function Measurements.

Cardiac function was measured by echocardiography at baseline, immediately after coronary occlusion and before sacrifice. Briefly, parasternal long- and short-axis views were obtained with both M-mode and 2-dimensional echo images. LV dimensions (LVEDD and LVESD) were measured perpendicular to the long axis of the ventricle at the midchordal level. LV ejection fraction and radial strain were calculated.

Local Intracoronary IGF-1/HGF Injection Preserves the Organization of the Infarcted Tissue and Improves Cardiomyocyte Survival after Acute Myocardial Infarction

Human recombinant IGF-I and HGF (hereafter abbreviated as IGF-1/HGF or GFs) were administered in differential doses to pigs by intracoronary injection 30 minutes after acute myocardial infarction. Additional pigs were injected with identical volume of saline alone, constituting the control group (CTRL).

The infarct size, as determined by planimetry, as a percent of the coronal circumferential area was not different between the GF-treated and CTRL group (28±5%, 26±7%, 29±5% in GF-1×, -2× and -4×, respectively, vs. 27±4% in CTRL).

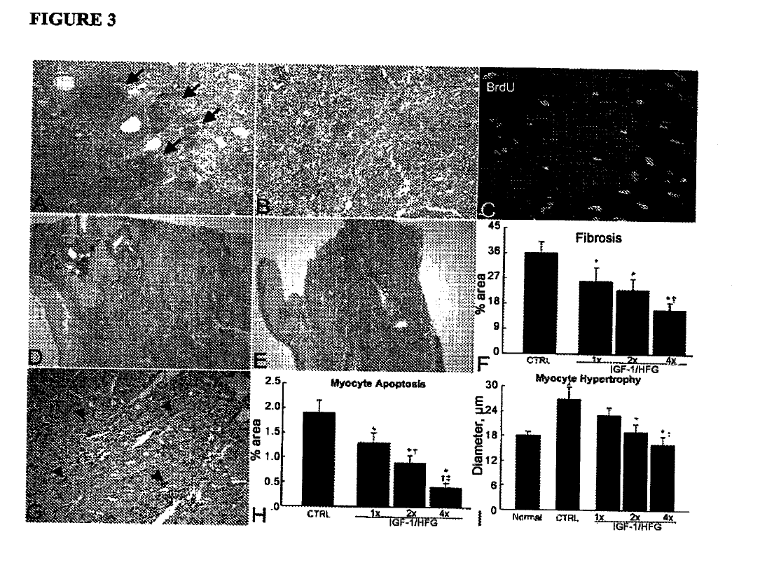

H&E and Sirius Red stained cross sections of the cardiac tissue in the remote, border and infarct zone revealed islands of survived myocardial tissue distributed amongst the fibrotic scar tissue in the infarct zone. These survived myocardial islands were much more abundant in the infarcted area of the GF-treated myocardium than in the CTRL-treated animals (FIG. 3A-B). Double immunofluorescence staining for α-sarcomeric actin and BrdU of the sections analyzed by confocal microscopy revealed that these islands consisted mainly of large α-sarcomeric actin positive, BrdU negative cardiomyocytes, a phenotype that confirmed their survival as pre-infarct myocardium and their mature, even hypertrophic nature (FIG. 3C). Furthermore, the GF-treated pig hearts had significantly less fibrotic tissue in the infarct region, compared to CTRL (FIG. 3D-F). More interestingly, this decrease exhibited a positive linear relationship with the dose of GF administered (FIG. 3F).

The study was not specifically geared to monitor the effect of the GF therapy on early cell death. However, from the results presented hereafter, it is clear that myocyte death continues to be very high in the peri-infarct/border zone a long time after the coronary occlusion/reperfusion event. This is likely due to the effects of pathological remodeling, which is known to establish a vicious circle between morphological adaptation and continued cell death. As shown in FIG. 3G-H, IGF-I/HGF administration significantly reduced late myocyte death in a dose dependent manner, as shown by a decrease in the number of myocytes positive for activated caspase-3, compared to CTRL. Consistent with the preservation of the anatomic morphology, myocyte survival and decreased remodeling, the GF-treated hearts exhibited a decreased myocyte hypertrophic response when compared to CTRL (FIG. 31). Taken together these findings indicate that IGF-1/HGF administration after acute MI has an important effect in preserving cardiomyocyte number and myocardial wall structure, reducing load on the surviving myocytes, which results in improved myocardial remodeling and decreased stimulus for myocyte death and hypertrophy of the surviving myocardium.

Intracoronary Administration of IGF-1/HGF after Acute Myocardial Infarction Activates the Resident pCSCs

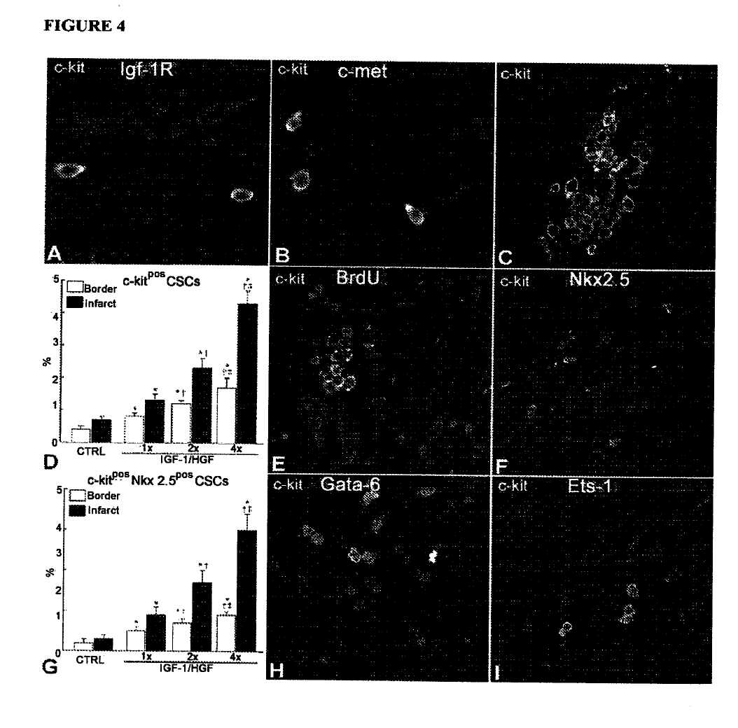

In normal (not shown) and post-MI hearts, ˜90% c-kit^{pos} pCSCs in situ express IGF-I and c-met (HGF) receptors as detected in by immunohistochemistry (FIG. 4A-B). Accordingly, the GF-treated infarcted pig hearts show a significant increase in the number of c-kit^{pos} pCSCs in the border region and even higher in the infarcted area, 21 days after MI (FIG. 4C-D). That this increase in c-kit^{pos} pCSCs is the result of GF administration is confirmed by its direct correlation to the GF-dose administered (FIG. 4D). At the highest GF dose, the number of c-kit^{pos} pCSCs in the infarcted area is >6-fold higher than in the CTRL hearts (FIG. 4D, SupplTable). Moreover, the linear increase between the I× and the 4× doses indicates that we have not reached a saturating dose to produce the maximum regenerative response. Many of the pCSCs were BrdU positive, a fixture that documents their birth after the production of the MI (FIG. 4E). Their cycling nature was confirmed by Ki-67 staining, which marks cells that are or have recently been in the cell cycle (data not shown). Many c-kit^{pos} cells expressed the transcription factors Nkx-2.5, Ets-1 or Gata⁶ indicative of their differentiation toward the main cardiac lineages, i.e. myocyte, endothelial and smooth muscle cells (FIGS. 4F-I). Quantitative analysis revealed that the number of c-kit^{pos}Nkx2.5^{pos} cells (committed myocyte/vascular precursors), significantly increased in the infarct and border regions in GF-treated pig hearts in a GF-dose dependent manner (FIG. 4G), reaching levels which were >10-fold higher than in CTRL hearts.

IGF-1/HGF Treatment Produces Robust Myocardial Regeneration after Acute Myocardial Infarction

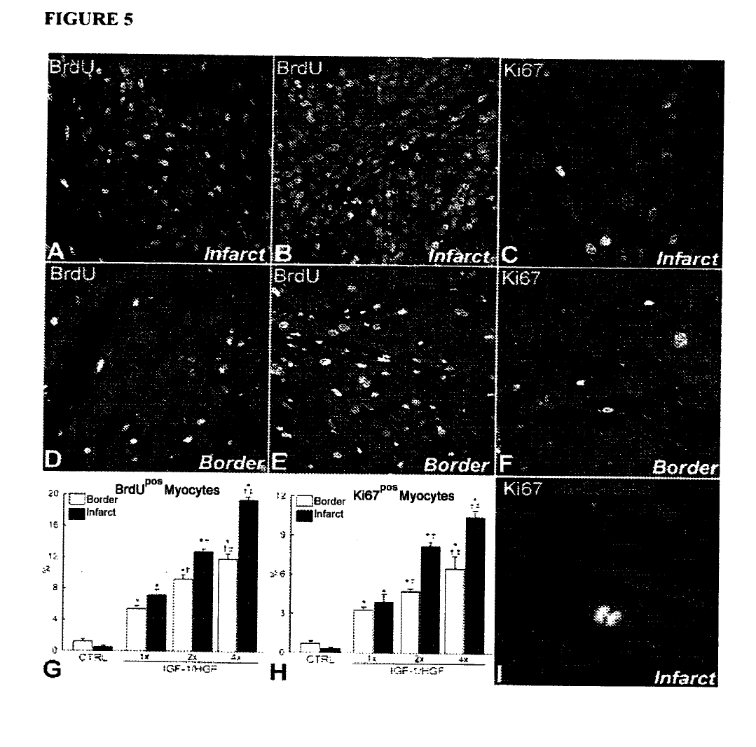

The GF-treated hearts, both in the infarct and peri-infarct/border regions, harbored a large population of very small, newly formed BrdUP^{pos} myocytes that had not yet reached the terminally differentiated state (FIG. 5). These data were confirmed by the expression of Ki67 in the small newly formed myocytes (FIGS. 5C and F), some of which were in mitosis and cytokinesis, confirming their immature nature (FIG. 51). Newly formed BrdUP^{pos} myocytes were also present in the peri-infarct/border region of the untreated saline-injected CTRL pigs. However, their number was—1/10 of the treated hearts and they were practically absent in the infarct zone (FIG. 5).

As it was the case for the pCSCs, there was a direct correlation between the number of small BrdU^{pos}/Ki67^{pos} newly formed myocytes with GF-dose, both in the infarct and border regions (FIG. 5G-H). In the GF-treated myocardium, the small BrdUP^{pos} myocytes were organized as clusters of regenerating bands in the infarct zone.

These regenerating bands were more organized in structure, and more compact and dense with increasing GF dose (FIG. 5A-B). Finally, neither the number nor the appearance of newly formed myocytes (the BrdUP^{pos} or Ki67^{pos}) in the distal region from the infarct (the spared myocardium) was not significantly different between GF-treated and CTRL animals (data not shown).

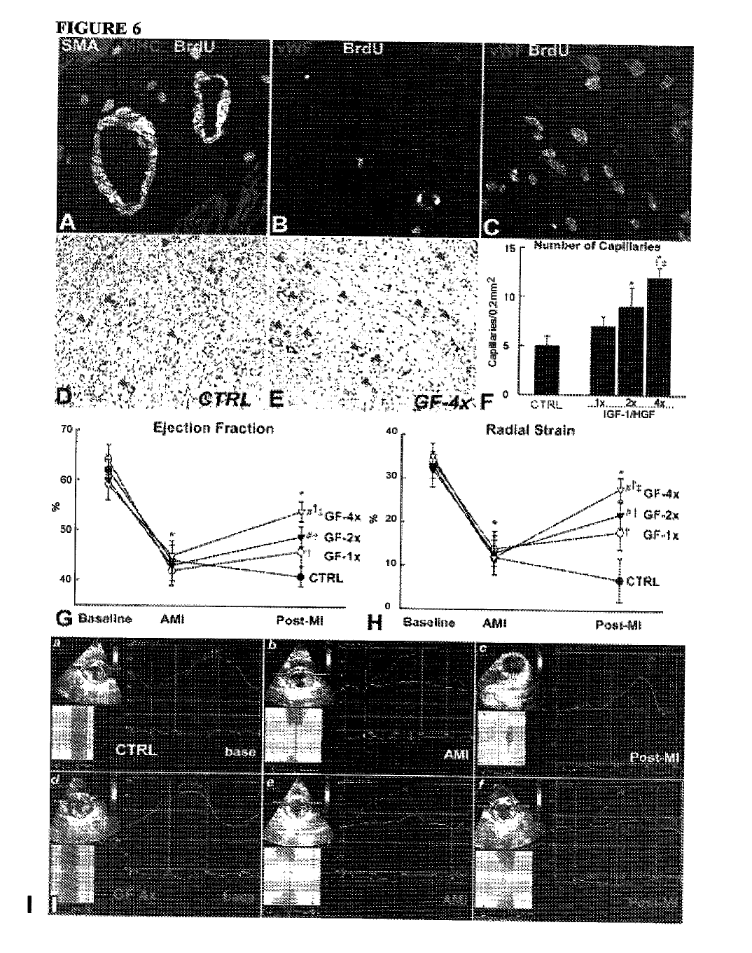

Newly formed BrdU-positive vascular structures were also evident in the border and infarcted myocardium (FIGS. 6 A-C). GF-treated hearts displayed increased number of capillaries and arterioles in the infarct zone, compared to saline-treated CTRL and this response was dose dependent (FIGS. 6D-F). Interestingly, new micro-vessels were most evident surrounding the survived islands of myocardium within the infarcted zone mentioned above which also had a higher density of newly formed small BrdU^{Pos} myocytes and regenerating bands (Gandia, C. et al., 2008. Stem Cells 26:638). This organization suggests the production of cardiopoietic (Behfar, A. et al., 2007. J. Exp. Med. 2007 204: 208) factors by the adult spared myocytes acting on the pCSCs.

The regenerated myocytes in the infarct zone at 21 days after MI were immature as demonstrated by their average size, as well as by the fact the many of them were still cycling as demonstrated by the expression of Ki-67 (FIG. 51F). In agreement with the suggested role for the cardiopoietic role of the mature myocytes, newly formed myocytes in contact or close proximity with mature ones (i.e. in the border zone) are of significantly larger size than those in the middle of the scar with no proximity to spared tissue (FIG. 5). It is also evident that GF-treatment plays a role in myocyte maturation as shown by the increased average myocyte size with increased GF dose.

Given the size of the porcine heart and the volume of the infarcted area, it is not possible to determine with any accuracy either the number of myocytes lost or the number of myocytes regenerated by the GF treatment. Nevertheless, careful sampling of the infarcted zone and the peri-infarct/border areas leaves no doubt that at 28 days the GF-treated infarcted heart has regenerated most of the lost myocytes, if not all.

### Example 10

### Intracoronary GF Administration Preserves and Might Improve Ventricular Function

Echocardiographic imaging showed that left ventricular ejection fraction (LVEF) was significantly depressed in CTRL and GF-treated pigs following coronary occlusion (FIG. 6G). However, 28 days after AMI, LVEF worsened slightly in CTRL, while it was significantly preserved/improved by the GF-treatment, when compared to CTRL (FIG. 6G). In order to gain further insight in regional cardiac function, tissue Doppler echocardiography was employed to measure antero-septal radial strain that was significantly improved in GF-treated pigs, compared to CTRL (FIGS. 6H-I). Cardiac function preservation/improvement correlated with increasing GF dose (FIG. 6).

### Example 11

### Intracoronary Administration of Up to 50 μg of IGF-I Encapsulated in 15 μm Diameter PLGA microspheres does not spill over into the systemic circulation

As demonstrated by Example #5, ≦99% of the 15 μm diameter microspheres are trapped into the capillary network of the target tissue, and specifically the myocardium. These data, however, do not address the issue of whether when the active molecule is unloaded is retained within the tissue or whether it leaches out into the capillary circulation and the venous return. To explore this issue, 5×10⁶ microspheres loaded with a total of 50 μg of rhIGF-1 were administered intracoronary at the origin of the left anterior descending artery following the same administration protocol outlined in Examples #5-7. The main different was that a catheter was left into the coronary sinus throught the jugular vein. During the administration, three hours after the procedure and then every 12 hours for the next 3 days blood samples were collected from the coronary sinus and the venus blood through an ear vein. Serum was prepared and the samples frozen in LN2 until the completion of the collection. All the samples were analyzed by ELISA employing human IGF-I detection kit (R&D, Minneapolis, Minn., USA) which does not cross-react with the porcine IGF-I. None of the samples either from the coronary sinus or from the systemic venous return scored positive. In our hands the minimal detection limits of the assay were 52.5 ng/ml for IGF-I. Therefore, although it is possible that some leakage below the detection levels of the ELISA occurred, it is clear that the majority of the IGF-I never left the myocardium.

### Example 12

### Intra-Arterial Local Administration of IGF-1/HGF to Damaged Skeletal Muscle Induced the Activation of the Muscle Stem Cells and Stimulates Regeneration

To test whether the protocol used to treat the damaged myocardium was effective in the treatment of other tissues, the same protocol was used to treat the post-ischemic skeletal muscle of the right leg of 3 pigs in which ischemic damaged had been produced by a 45 min complete balloon occlusion of the femoral artery. As in the case of the myocardium, after a 30 min reperfusion by deflation of the balloon, a suspension of 20 mL of PBS containing IGF-I and HGF microspheres of 15 μm diameter, prepared as described in example #2 for a total dosis de 8 μm of IGF-I and 2 μm of HGF. The animals were sacrificed 3 weeks later and biopsies of the quadriceps muscle analyzed by immunohistology to determine the degree of activation of the stem cells in the lesion.

As described for the myocardium, after the occlusion of the femoral artery the animals were implanted an osmotic pump to continuously deliver a solution of BrdU known to efficiently label all replicating cells. In this manner all cells born after the start of the therapy are BrdU label, which allows for a comparison of the regenerative reaction between the controls and the treated animals. In each case the quadriceps of the left leg served as undamged control.

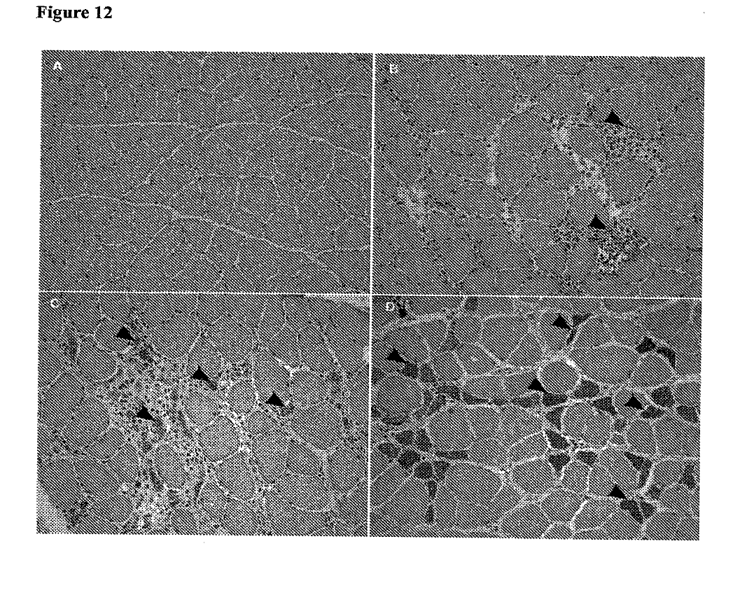

As shown in FIG. 12, and Table 6, the local administration of IGF-1/HGF encapsulated in PLGA microspheres of 15 μm in diameter was very effective in stimulation the regeneration of muscle tissue in the treated leg but not in the contralateral one as compared with the ischemic but placebo treated controls.

TABLE 6
Skeletal Muscle Regeneration in Response to Local
Administration of Growth Factors
# of BrdU labeled myofiber
nuclei per 1 × 10³ myofiber nuclei

| Animal # | Damaged leg | Contralateral leg |
|---|---|---|
| 1 | 337 | 17 |
| 2 | 289 | 22 |
| 3 | 364 | 13 |

Conclusion:

The local administration of growth factors to damaged tissue others than the myocardium has a stimulatory effecto in the regenerative reaction of the damaged tissue which is localized to the area downstream from the site of administration of the microspheres, as is expected for a delivery system that targets the capillary network of the damaged tissue/organ.

### Example 13

### Intracoronary Injection of IGF-1/HGF/SCF has a More Potent Effect in the Activation of the CSCs and Preserving Ventricular Function than IGF-1/HFG Alone

To test whether the addition of new factors to the protocol described in the previous Examples would improve the regenerative reaction of the post-infarcted myocardium, a group of 3 animals were administered the higher doses of IGF-I (8 μg) and HGF (2 μg) used in example #9 together with 4 μg of SCF. Each of these factors was encapsulated in PLGA microspheres of 15 μm diameter as described in Example #2. The protocol for the production of the infarct, monitoring and the administration of the microsphere suspensions was as described in Examples #5-7. The animals were sacrificed at 4 weeks after the treatment.

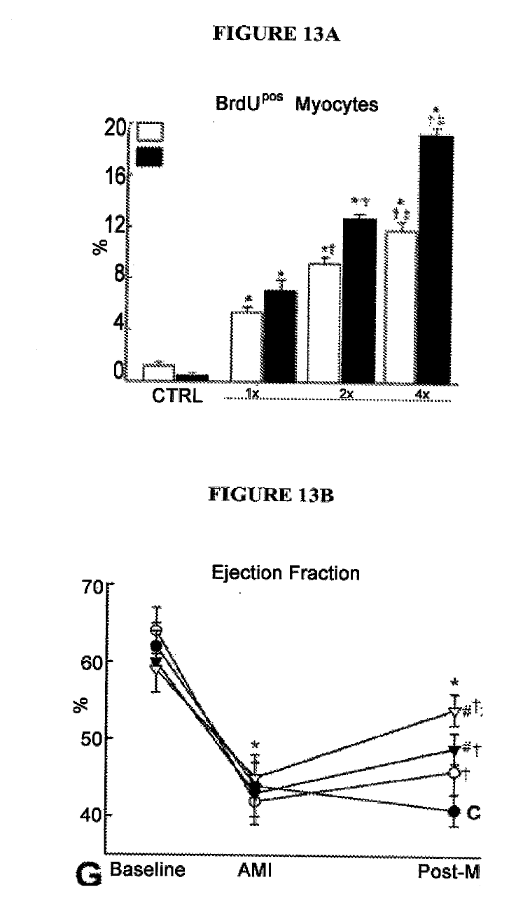

As shown in FIG. 13A and FIG. 13B, the regeneration produced by the three factors protocols is significantly better in both the level of regeneration as well as in the maturation of the regenerated myocytes that by the combination of IGF-1/HGF. The cellular and histological, and functional parameters confirms the synergy among the factors employed and documents the suitability of the described invention to produce multiple variants of the therapeutic compound to modify the regenerative reaction. It is reasonable to extrapolate from these data that in addition to the addition or subtraction of particles with particular factors, other variations might involve changing the dose of a particular factor or set of factors, the profile of release/unloading for a particular factor, the degree of loading, etc.

### Conclusion:

The present invention allows for the formulation of an almost infinity number of specific combination of therapeutic compounds starting from a limited set of building blocks in which each factor can be used at different doses, different patterns of release and combined with an unlimited of other factors. This allows in a single administration to target a particular tissue with different combinations of therapeutic agents each of which might act at a different time, on a different cell target, and require a different effective dose. These possibilities are particularly advantageous for tissues of difficult access which can not be accessed repeatedly, such as the myocardium and most of the internal organs.

The effects of insulin-like growth factor-1 (IGF-1) and hepatocyte growth factor (HGF) were tested using an injectable supramolecular hydrogel based on ureido-pyrimidinone (UPy) moieties in a porcine model of chronic myocardial infarction (MI).

Preclinical data in rodents, dogs and pigs provided proof that IGF-1/HGF treatment could activate c-kit^{pos} CD45^{neg} epCSCs thereby giving rise to enhanced myocardial repair and regeneration in the acute phase of MI.

### Example 14

### Mortality and Procedural Data

Three animals died during the induction of ischemia by LCx occlusion as consequence of refractory ventricular fibrillation. One animal died four weeks later, prior to the intervention, shortly after induction of general anesthesia presumably because of cardiac failure. Of the survived 14 animals, 5 animals were randomly allocated to UPy-GF, 5 animals to GF and the remaining 4 animals to UPy hydrogel alone, serving as controls. One of the control animals was excluded from the analysis since there was no initial decline in cardiac function, a limited troponin rise after MI and only a minor endocardial rim of scar tissue visible by TTC staining. In one of the GF treated animals, histological analysis was not possible due to a technical error during the fixation process of the tissue samples.

### Example 15

### The UPy Hydrogel Carrier Prolongs IGF-1/HGF Release Whilst Maintaining its Bioactivity

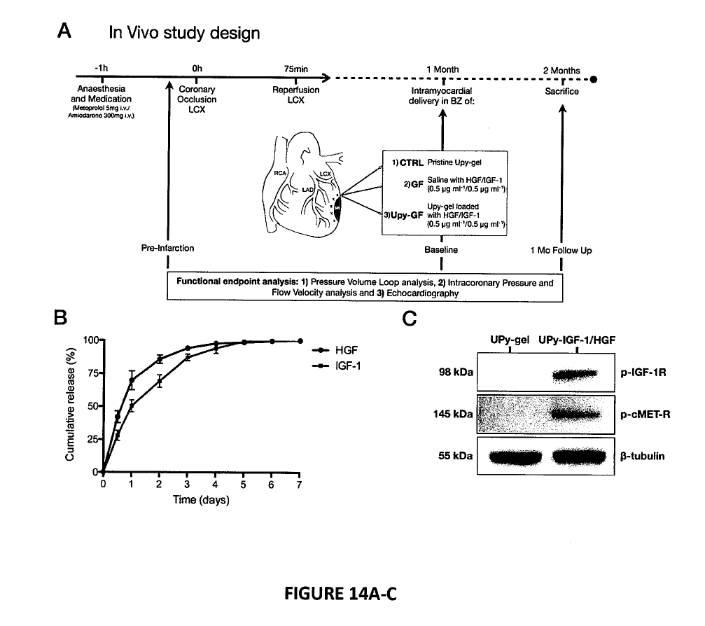

A schematic study design is depicted in FIG. 14A. Prior to the incorporation of the IGF-1/HGF, the UPy hydrogel (Sijbesma R P, Beijer F H, Brunsveld L, et al. Reversible polymers formed from self-complementary monomers using quadruple hydrogen bonding. Science 1997; 278:1601-4.; Dankers P Y W, Harmsen M C, Brouwer La, van Luyn MJa, Meijer E W. A modular and supramolecular approach to bioactive scaffolds for tissue engineering. Nature materials 2005; 4:568-74.) was made soluble by increasing its pH to approximately 9. First, the release kinetics of IGF-1/HGF in vitro was assessed for the UPy hydrogel (FIG. 14B). Both IGF-1 and HGF showed a similar sustained release over a four-day timespan. The release of HGF was characterized by an initial outburst of 42% compared to only 28% of the total IGF-1. Over the next three days, a gradual release pattern for both growth factors was observed (FIG. 14B). Next, we tested whether the increased pH could induce protein degradation and breakdown of the IGF-1 and/or HGF. Following 6 hours of incubation, UPy^{pH9} hydrogel released IGF-1/HGF still showed a preserved ability to activate their corresponding receptors, IGF1R for IGF-1 and c-MET-R for HGF (FIG. 14C).

Taken together, these data show that UPy^{pH9} hydrogel carrier acts as a sustained release platform whilst ensuring the preserved bioactivity of IGF-1/HGF.

### Example 16

### IGF-1/HGF Administration Improves Cardiac Function in Chronic MI

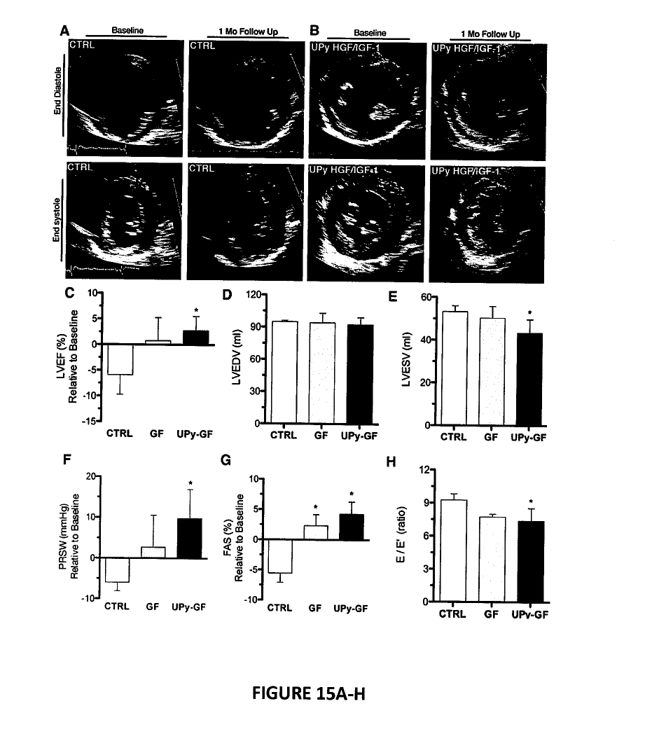

To test the effects of the GF treatment on cardiac function after chronic MI, PV loop analysis and echocardiography was assessed prior to coronary occlusion, at 1 month (prior to injections in the chronic MI) and at 2 months (1 month after injections) after MI. First, the controls, UPy hydrogel without growth factors, were compared against a historical cohort of identical MI procedure and NOGA injections with 0.9% saline 1 month after MI. There were no differences in any echocardiographic or PV-loop derived parameters (data not shown). Thus, with no indication that the UPy hydrogel by itself influenced post-MI remodeling, we considered the UPy hydrogel as controls. Fractional area shortening (FIGS. 15A to B) was significantly improved in both the GF and UPy-GF groups compared to the CTRL animals (FIG. 15G; ±2.3±1.8% vs +4.2±2.0% vs −5.6±1.5%; p<0.0001). Progressive deterioration in left ventricular ejection fraction was reversed in the UPy hydrogel release group (FIG. 15C; mean change +2.8±2.7%), compared to CTRL animals (FIG. 15C; −5.9±3.8%, p=0.001). However, there were no apparent signs of cardiac dilatation in all groups and LV end diastolic volume did not differ between treatment groups (FIG. 15D; p=0.873). However, UPy-GF resulted in significantly lower end systolic volumes 1 month after the treatment delivery (FIG. 15E; p=0.04).

With regard to diastolic function of the heart, the ratio of transmitral flow velocity to annular peak diastolic velocity (E/E′) was preserved in the IGF-1/HGF treated animals (FIG. 15H; GF 7.7±0.3; UPy-GF 7.4±1.1), compared to CTRLs (FIG. 15H; 9.3±0.6; p=0.04).

### Example 17

### Targeted Intramyocardial IGF-1/HGF Delivery Attenuates Cardiomyocyte Hypertrophy and Fibrosis in Chronic MI

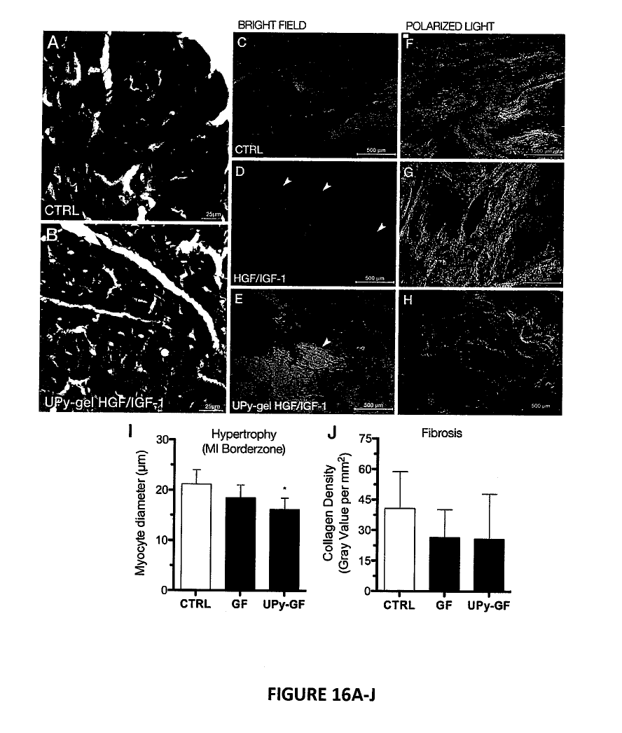

As a reference, average cardiomyocyte diameter in the non-infarcted pig heart in the absence of hypertrophy was ˜18 μm. Four weeks after the NOGA-guided injections, histological analysis revealed significant cardiomyocyte hypertrophy in the borderzone of the CTRL hearts (FIG. 16A). In contrast, both GF and UPy-GF treatment attenuated cardiomyocyte hypertrophy as well as increased the number of relatively small (<18 μm) cardiomyocytes, compared to CTRL (FIGS. 16B to C; GF 18.47±2.56 μm vs UPy-GF 16.04±1.85 μm vs CTRL 21.20±2.81 μm respectively; p=0.04). In addition, both the GF and UPy-GF treated hearts further displayed a non-significant trend towards reduction in fibrosis, shown by picric Sirius red staining (FIGS. 16C to H), compared to the CTRL group (p=0.53).

### Example 18

### Intramyocardial IGF-1/HGF Administration Leads to Formation of New Cardiomyocytes

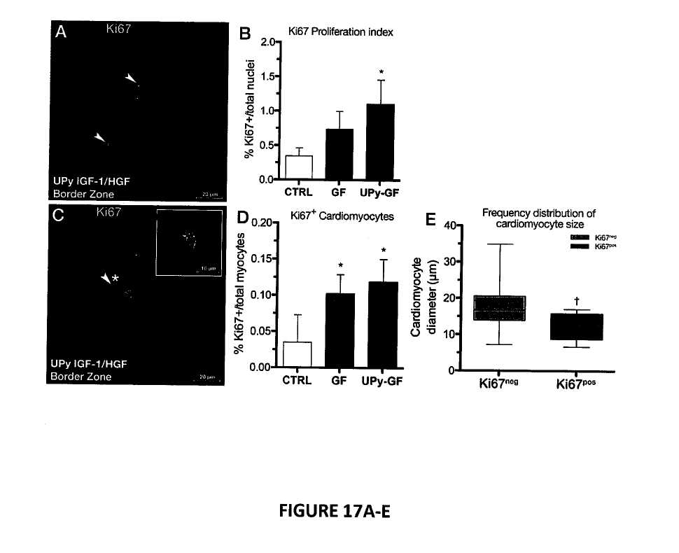

Different myocardial cell types express growth factor receptors for IGF-1 and/or HGF. Thus, we sought to investigate the level of cell proliferation in the borderzone of the chronic MI after GF treatment. Even 30 days after the injection procedure, an increased proliferation rate assessed by Ki67 expression was present within the GF treated hearts, which was greater in the UPy-GF treated hearts (FIGS. 17A,B). In particular, the borderzone of the GF and UPy-GF treated animals harbored newly formed, small, immature Ki67pos cardiomyocytes, which amounted to ˜1 every 1000 cardiomyocytes (FIG. 17C). These small Ki67pos cardiomyocytes accounted for >10% of the total proliferating Ki67pos cells, in the GF treated hearts, making their existence physiologically significant. Although Ki67pos cardiomyocytes were also observed in the CTRL hearts, these were only witnessed in ˜1 every 3000 cardiomyocytes (p=0.016). To verify that these Ki67pos cardiomyocytes were newly formed, we measured their size and compared this with Ki67neg cardiomyocytes. Indeed, the Ki67pos cardiomyocytes were on average smaller (FIG. 17E; 12.52±3.97 μm) compared to their Ki67neg counterparts (FIG. 17E; 17.48±3.85; p=0.0006), suggestive of a newly formed and immature cardiomyocyte subpopulation. (Beltrami A P, Barlucchi L, Torella D, et al. Adult cardiac stem cells are multipotent and support myocardial regeneration. Cell 2003; 114:763-76., Ellison G M, Torella D, Dellegrottaglie S, et al. Endogenous Cardiac Stem Cell Activation by Insulin-Like Growth Factor-1/Hepatocyte Growth Factor Intracoronary Injection Fosters Survival and Regeneration of the Infarcted Pig Heart. Journal of the American College of Cardiology 2011; 58).

### Example 19

### IGF-1/HGF Delivery Leads to the Formation of New Capillaries in the Infarct Borderzone

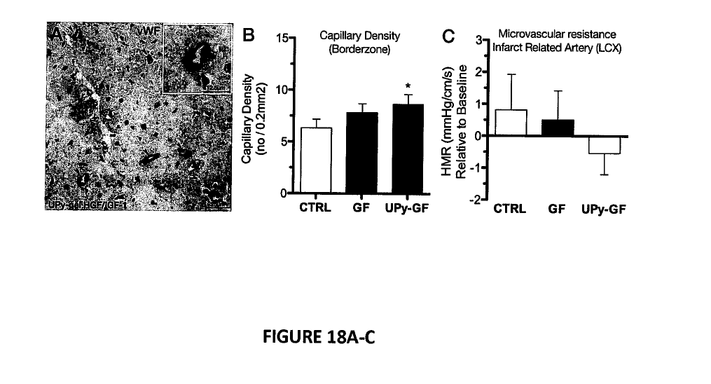

The IGF-1/HGF treatment led to an increased number of capillaries in the infarct borderzone, favoring the UPy-GF group (FIG. 18 A to B; UPy-GF 8.6±0.9/0.2 mm2 vs GF 7.8±0.9/0.2 mm2 vs CTRL 6.3±0.8/0.2 mm2 respectively; p=0.022). Consistent with the increased capillerisation, the hyperemic microvascular resistance index (HMR) (a simultaneously measured intracoronary pressure-/ and flow velocity derived parameter) was decreased in the infarct related artery in the UPy-GF group compared to the HMR value measured just prior to the intramyocardial treatment delivery (FIG. 18C; p=0.053).

### Example 20

### IGF-1/HGF Administration Leads to Expansionary Growth of the epCSC Compartment and Induces Cardiogenic Precursors

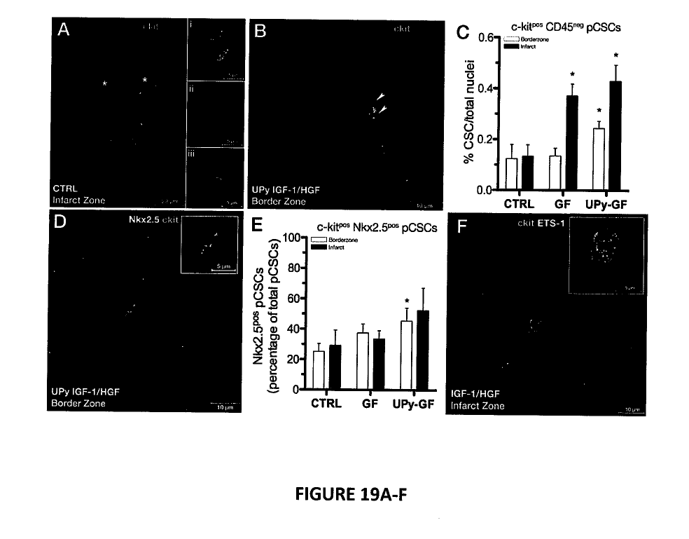

To elucidate potential mechanisms governed by IGF-1/HGF stimulation that are responsible for the observed new cardiomyocyte and capillary formation, we determined the number and precursor state of the previously described c-kit^{pos} CD45^{neg} epCSCs. (Ellison G M, Torella D, Dellegrottaglie S, et al. Endogenous Cardiac Stem Cell Activation by Insulin-Like Growth Factor-1/Hepatocyte Growth Factor Intracoronary Injection Fosters Survival and Regeneration of the Infarcted Pig Heart. Journal of the American College of Cardiology 2011; 58) We found increased c-kit^{pos} cells in the infarct and borderzone with GF treatment, however ˜73% of all c-kit^{pos} cells also co-expressing CD45, identifying cardiac mast cells (FIG. 19Aiii; Suppl. FIG. 3).(7) Furthermore, there was an infiltration of CD45^{pos} c-kit^{neg} cells into the infarct and borderzone (FIG. 19Aii). c-kit^{pos} CD45^{neg} epCSCs (FIGS. 19Ai;B) had a relatively small cytoplasm to nuclei ratio and in the infarct zone, the total number of epCSCs was increased four-fold by IGF-1/HGF delivery, compared to CTRL hearts (FIG. 19C; 0.37±0.09% vs 0.43±0.14 vs 0.12±0.07% respectively, p=0.004). With regard to the borderzone, the highest increase in c-kit^{pos} epCSC number was observed in the UPy-GF group (FIG. 19C; 0.24±0.06%) compared to GF or CTRL hearts (FIG. 19C; 0.14±0.06% vs 0.12±0.01%, p=0.03). Of those epCSCs, sustained IGF-1/HGF release induced a modest increase in the number of progenitor epCSCs (˜40%) that co-expressed the early cardiac transcription factor Nkx2.5, indicative of their commitment towards the cardiomyogenic lineage (FIG. 19D to E). Furthermore, another subset of epCSCs expressed the transcription factor Ets-1, indicative of their commitment to the endothelial lineage, and the generation of capillaries lineage (FIG. 19F).

### Discussion

The functional and histological/cellular effects of intramyocardial administration of IGF-1/HGF in chronic MI were investigated. We show that improved delivery of IGF-1/HGF by UPy-hydrogel holds potential as a novel treatment for chronic MI. Four weeks after delivery, UPy-IGF-1/HGF treatment led to a reduction in pathological cardiac remodelling, activated and increased the number of epCSCs, and led to the formation of new cardiomyocytes and capillaries Importantly, the repair and regeneration of the damaged myocardial tissue was associated with a significant improved cardiac function.

Heart Regeneration and eCSCs

To date, the presence of endogenous mechanisms for cardiomyocyte renewal in the post-natal heart remains a subject of intense debate. (Laflamme Ma, Murry C E. Heart regeneration. Nature 2011; 473:326-35). Our findings presented here challenge the prevalent view that the adult mammalian heart, at best, can only increase its myocyte volume by means of a hypertrophic response of existing cardiac myocytes in the absence of new myocyte formation. Here, we show that the adult infarcted pig heart contains immature cardiac myocytes that are substantially smaller than normal, non-hypertrophied, myocytes and do not reside in the quiescent G0 phase of the cell cycle, as would be expected given the hypothesis that the heart is a post-mitotic organ. Importantly, this regenerative potential of the adult heart could be effectively boosted by sustained release of the growth factors IGF-1 and HGF. These findings further ascertain the definitive presence of cardiomyocyte renewal in the adult mammalian heart as deducted from elaborate pulse-chase experiments published by various independent research groups. (Bergmann O, Bhardwaj R D, Bernard S, et al. Evidence for cardiomyocyte renewal in humans. Science (New York, N.Y.) 2009; 324:98-102, Hsieh P C H, Segers V F M, Davis M E, et al. Evidence from a genetic fate-mapping study that stem cells refresh adult mammalian cardiomyocytes after injury. Nature medicine 2007; 13:970-4, Senyo S E, Steinhauser M L, Pizzimenti C L, et al. Mammalian heart renewal by pre-existing cardiomyocytes. Nature 2012:2-6, Malliaras K, Zhang Y, Seinfeld J, et al. Cardiomyocyte proliferation and progenitor cell recruitment underlie therapeutic regeneration after myocardial infarction in the adult mouse heart †. 2012:1-60).

Two mechanisms have been put forward to play a significant role in myocardial regeneration (i) dedifferentiation of pre-existing mature cardiomyocytes, followed by proliferation of these dedifferentiated cells and subsequent differentiation (Senyo S E, Steinhauser M L, Pizzimenti C L, et al. Mammalian heart renewal by pre-existing cardiomyocytes. Nature 2012:2-6, Malliaras K, Zhang Y, Seinfeld J, et al. Cardiomyocyte proliferation and progenitor cell recruitment underlie therapeutic regeneration after myocardial infarction in the adult mouse heart †. 2012:1-60, Bersell K, Arab S, Haring B, Kühn B. Neuregulin1/ErbB4 signaling induces cardiomyocyte proliferation and repair of heart injury. Cell 2009; 138:257-70); (ii) the presence of stem/progenitor cells that create progeny that both maintain the stem cell pool as well as differentiate towards various cell types including cardiac myocytes and new vasculature. (Goumans M-J, de Boer T P, Smits A M, et al. TGF-β1 induces efficient differentiation of human cardiomyocyte progenitor cells into functional cardiomyocytes in vitro. Stem cell research 2007; 1:138-49, Beltrami A P, Barlucchi L, Torella D, et al. Adult cardiac stem cells are multipotent and support myocardial regeneration. Cell 2003; 114:763-76, Ellison G M, Torella D, Dellegrottaglie S, et al. Endogenous Cardiac Stem Cell Activation by Insulin-Like Growth Factor-1/Hepatocyte Growth Factor Intracoronary Injection Fosters Survival and Regeneration of the Infarcted Pig Heart. Journal of the American College of Cardiology 2011; 58, Malliaras K, Zhang Y, Seinfeld J, et al. Cardiomyocyte proliferation and progenitor cell recruitment underlie therapeutic regeneration after myocardial infarction in the adult mouse heart †. 2012:1-60, Urbanek K, Quaini F, Tasca G, et al. Intense myocyte formation from cardiac stem cells in human cardiac hypertrophy. Proceedings of the National Academy of Sciences of the United States of America 2003; 100, Urbanek K, Torella D, Sheikh F, et al. Myocardial regeneration by activation of multipotent cardiac stem cells in ischemic heart failure. Proceedings of the National Academy of Sciences of the United States of America 2005; 102).

The combination of these two mechanisms played a role in myocardial regeneration following exogenous delivery of CSCs in rodents with myocardial infarction. (Malliaras K, Zhang Y, Seinfeld J, et al. Cardiomyocyte proliferation and progenitor cell recruitment underlie therapeutic regeneration after myocardial infarction in the adult mouse heart †. 2012:1-60). Our present findings document that following IGF-1/HGF administration, the number of resident c-kit^{pos} epCSCs in the peri-infarcted area increased analogously to the increase in the presence of newly formed, immature, Ki67pos cardiomyocytes. This reinforces the likelihood that these eCSCs play a role in cardiac repair and regeneration following ischemic injury. Indeed, some eCSCs in the peri-infarct region also co-expressed the nuclear transcription factors Nkx2.5 and Ets-1, indicative of their commitment towards the myogenic and vasculature lineage, respectively. Stem cell based tissue-/cellular homeostasis in the heart does not seem to differ from other organs previously regarded incapable of self-renewal, such as the brain (Doetsch F. A niche for adult neural stem cells. Curr Opin Genet Dev 2003; 13:543-50, Doetsch F, Caille I, Lim D A, Garcia-Verdugo J M, Alvarez-Buylla A. Subventricular zone astrocytes are neural stem cells in the adult mammalian brain. Cell 1999; 97:703-16) or the skeletal muscle. (Kuang S, Kuroda K, Le Grand F, Rudnicki M A. Asymmetric self-renewal and commitment of satellite stem cells in muscle. Cell 2007; 129:999-1010, Collins C A, Olsen I, Zammit P S, et al. Stem cell function, self-renewal, and behavioral heterogeneity of cells from the adult muscle satellite cell niche. Cell 2005; 122:289-301).

### Growth Factors to Stimulate Endogenous Cardiac Repair

Recently, essential growth factor/signaling pathways for cardiomyogenesis during the embryonic period have been summarized. (Noseda M, Peterkin T, Simoes F C, Patient R, Schneider M D. Cardiopoietic factors: extracellular signals for cardiac lineage commitment. Circ Res 2011; 108:129-52). Various growth factors have been identified as potential candidates to guide post-natal stem-progenitor cells towards a cardiomyogenic fate. (Linke A, Müller P, Nurzynska D, et al. Stem cells in the dog heart are self-renewing, clonogenic, and multipotent and regenerate infarcted myocardium, improving cardiac function. Proceedings of the National Academy of Sciences of the United States of America 2005; 102:8966-71, Ellison G M, Torella D, Dellegrottaglie S, et al. Endogenous Cardiac Stem Cell Activation by Insulin-Like Growth Factor-1/Hepatocyte Growth Factor Intracoronary Injection Fosters Survival and Regeneration of the Infarcted Pig Heart. Journal of the American College of Cardiology 2011; 58, Hahn J Y, Cho H J, Kang H J, et al. Pre-treatment of mesenchymal stem cells with a combination of growth factors enhances gap junction formation, cytoprotective effect on cardiomyocytes, and therapeutic efficacy for myocardial infarction. J Am Coll Cardiol 2008; 51:933-43; Takehara N, Tsutsumi Y, Tateishi K, et al. Controlled delivery of basic fibroblast growth factor promotes human cardiosphere-derived cell engraftment to enhance cardiac repair for chronic myocardial infarction. J Am Coll Cardiol 2008; 52:1858-65; Roggia C, Ukena C, Bohm M, Kilter H. Hepatocyte growth factor (HGF) enhances cardiac commitment of differentiating embryonic stem cells by activating PI3 kinase. Experimental cell research 2007; 313:921-30).

The possibility was raised that eCSCs are not just mere consumers of growth factors, but actively secrete a wide range of growth factors themselves, providing intricate networks of auto-/ and paracrine feedback loops. (Chimenti I, Smith R R, L1 T-S, et al. Relative roles of direct regeneration versus paracrine effects of human cardiosphere-derived cells transplanted into infarcted mice. Circulation research 2010; 106:971-80). Since we have observed effects on cell proliferation even detectable one month after delivery of a single dose of IGF-1/HGF, the activation and increase of the c-kitpos eCSC compartment itself could form a necessary chain in the link of a growth factor mediated feedback loop that leads to sustained epCSC activation and proliferation and resultant cardiomyocyte formation, long after the primary stimulus has disappeared. (Ellison G M, Torella D, Dellegrottaglie S, et al. Endogenous Cardiac Stem Cell Activation by Insulin-Like Growth Factor-1/Hepatocyte Growth Factor Intracoronary Injection Fosters Survival and Regeneration of the Infarcted Pig Heart. Journal of the American College of Cardiology 2011; 58).

### Sustained Release of GF Using a Bioscaffold

In this present work, the additional therapeutic value provided by the sustained release of IGF-1/HGF using the UPy hydrogel carrier was also addressed. None of the reported outcome measures showed statistical significance between the sustained growth factor release by UPy hydrogel compared to equal concentrations of IGF-1/HGF dissolved in saline. However, there is a highly consistent pattern visible that the UP-GF treated animals outperformed the GF treated animals on all levels of outcome measures (i.e. cardiomyocyte formation, number of c-kitpos eCSCs, cardiac function). Previous proof of concept experiments validating the UPy hydrogel showed that the hydrogel created a successful gradient of growth factors (data not shown). In line with these findings, similar work with an alginate based hydrogel reinforces the rationale to use smart biomaterials to improve the effects of growth factor administration therapy (Ruvinov E, Leor J, Cohen S. The promotion of myocardial repair by the sequential delivery of IGF-1 and HGF from an injectable alginate biomaterial in a model of acute myocardial infarction. Biomaterials 2010:1-14).

### Clinical Perspective

By avoiding myocardial biopsies to extract eCSCs that need ex vivo up scaling to acquire clinically relevant numbers for subsequent delivery, one escapes from several drawbacks of cellular products as a novel treatment for ischemic heart disease. (Nadal-Ginard B, Torella D, Ellison G. [Cardiovascular regenerative medicine at the crossroads. Clinical trials of cellular therapy must now be based on reliable experimental data from animals with characteristics similar to human's]. Rev Esp Cardiol 2006; 59:1175-89, Torella D, Ellison G M, Karakikes I, Nadal-Ginard B. Resident cardiac stem cells. Cell Mol Life Sci 2007; 64:661-73). First and foremost, cellular therapy requires dedicated clinical centers that have both the expertise and high-cost resources for isolating, culturing and handling of the stem cell products to pursue cardiac repair. Secondly, it relies on an available time-span necessary for culturing stem/progenitor cells that is not present as in the case of acute myocardial infarction. Therefore, in situ activation of the endogenous CSC compartment could bypass the aforementioned limitations of exogenous stem cell therapy. This holds true in particular for the chronic MI patients, in which aging and co-morbidities also reduce the potency of the eCSC compartment. One particular aspect is the dramatic increase in cellular senescence of eCSCs to ˜70% of all eCSCs in aged mice. Growth factors such as IGF-1 are capable to reverse this process in aged mice and restore function in these dysfunctional eCSCs.

Previous work on the therapeutic efficacy of IGF-1/HGF relied on transepicardial injections during open-chest surgery as the route of delivery. (Linke A, Müller P, Nurzynska D, et al. Stem cells in the dog heart are self-renewing, clonogenic, and multipotent and regenerate infarcted myocardium, improving cardiac function. Proceedings of the National Academy of Sciences of the United States of America 2005; 102:8966-71, Ruvinov E, Leor J, Cohen S. The promotion of myocardial repair by the sequential delivery of IGF-1 and HGF from an injectable alginate biomaterial in a model of acute myocardial infarction. Biomaterials 2010:1-14, Urbanek K, Rota M, Cascapera S, et al. Cardiac stem cells possess growth factor-receptor systems that after activation regenerate the infarcted myocardium, improving ventricular function and long-term survival. Circulation research 2005; 97:663-73). In contrast, we used a percutaneous approach with the NOGA catheter system to acquire information on the infarct location and used the MYOSTAR catheter for targeted intramyocardial delivery in the peri-infarct/borderzone of the chronic MI. As a consequence, the entire study protocol employed in this present work is clinically feasible and can be performed at a conventional catheterization laboratory.

### CONCLUSION

In summary, four major conclusions can be deducted from this study: (1) targeted intramyocardial IGF-1/HGF injections attenuated pathologic cardiac remodeling and increased the formation of small newly formed cardiomyocytes in the borderzone of the infarct scar, in the post-MI adult pig heart; (2) IGF-1/HGF admission gave rise to a robust increase of the c-kitpos epCSC compartment of the heart and increased their commitment towards the cardiomyogenic and vasculature lineage; (3) the use of a smart hydrogel carrier that acts as a sustained release platform increased the effectiveness of growth factor therapy as a treatment for chronic MI; (4) intramyocardial IGF-1/HGF injections in the borderzone of the infarct scar led to an improvement in cardiac systolic and diastolic function when compared to control treated hearts, as measured by 3-dimensional echocardiography. These findings identify the UPy hydrogel carrier system as a practical, affordable and widely applicable therapeutic strategy designated to counteract the adverse remodeling and natural disease progression in the post-MI heart that would otherwise lead to congestive heart failure.

In the chronic MI, intramyocardial UPy-IGF-1/HGF injections reduced pathological cardiomyocyte hypertrophy (p=0.04). The IGF-1/HGF led to the formation of new, small cardiomyocytes (p=0.016) and increased capillerisation (p=0.022). The c-kit^{pos} CD45^{neg} epCSC population was increased almost fourfold in the borderzone of the UPy-IGF-1/HGF treated hearts compared to CTRL hearts (p=0.023). Functionally, LV ejection fraction was improved in the UPy-IGF-1/HGF animals (52.9±2.8%) compared to IGF-1/HGF (46.6±1.5%) and CTRLs (43.9±3.6%; p=0.001). The delivery of IGF-1/HGF by UPy-hydrogel is a new and above all clinically feasible treatment protocol for chronic MI that reduced pathological cardiac hypertrophy, increased epCSC number and formation of new cardiomyocytes and capillaries leading to improved cardiac function in a porcine model of chronic MI.

### FIGURE LEGENDS

FIG. 1. Distribution and Characterization of c-Kit^{Pos} Cardiac Cells in the Adult Pig Heart.

(A-B) Representative confocal images of c-kit positive (c-kit^{pos} white) cells in the right atria (A) and left ventricle (B) of the normal pig heart. Cardiomyocytes stained in red (shown in grey in the figures) by α-sarcomeric actin α-sarc act) and nuclei stained with DAPI in blue. (C) c-kit^{pos} cells are distributed throughout the atrial and ventricular myocardium with a higher density in the atria and the apex, compared to Right and left ventricle (RV, LV). *p<0.05 vs RV and LV. (D) Representative FACS analysis of c-kit^{pos} cells within the myocyte-depleted cardiac cell population for the atria, ventricle (RV), and apex. (E) c-kit^{pos} cells obtained using MACS show >90% enrichment. FACS analysis of c-kit^{pos} enriched porcine cardiac cells revealed that they are negative for hematopoietic cell lineage markers CD45 and CD34. Also, a high fraction of c-kit^{pos} porcine cardiac cells express the mesenchymal cell lineage markers, CD90 and CD 166.

### FIG. 2 c-kit^{pos} Porcine Cardiac Cells Express Sternness Markers, have Stem Cell Properties of Clonogenicity, Self-Renewal, Cardiosphere-Forming and Multipotency, and Express Intact Signaling IGF-1/HGF Systems Modulating their Activation

(A) A light microscopy image showing expanded c-kit^{pos} porcine cardiac cells at the 4th passage. (B) A light microscopy image of a clone, after single c-kit^{pos} porcine cardiac cells were deposited into wells of terasaki plates to generate single cell clones. (C) The clonogenicity of c-kit^{pos} porcine cardiac cells was similar across cardiac chambers, and compared to mouse and rodent CSCs. (D) Immunofluorescent staining of cloned c-kit^{pos} porcine cardiac cells confirmed the expression of c-kit (white), and revealed the expression of FIk-I, Oct-4, Nanog, Tert, Bmi-1, Nkx2.5 and IsI-I (all shown in grey), which indicates they are a mixture of cardiac stem and progenitor cells. Images are 2O× magnification, with zoom captures as inset. (E) Cloned c-kit^{pos} porcine cardiac cells formed cardiospheres (a). When c-kit^{pos} (white) cardiospheres (b) were placed in laminin-coated dishes in cardiogenic medium, cardiosphere cells spread out from the sphere (c). Four to six days later, cells on the periphery of the sphere increased expression of biochemical markers for cardiomyocytes (α-sarcomeric actin, α-Sarc Act; d), smooth muscle (Smooth Muscle Actin, SMA; e), and endothelial (von Willebrand factor, vWF; f) cells (all shown in grey fluorescence). (F) Immunofluorescent staining shows that c-kit^{pos} porcine CSCs have IGF-I and HGF receptors (grey, Igf-IR and c-met, respectively). (G-H) When grown in culture, freshly isolated porcine c-kit^{pos} cardiac cells respond to the stimulation of IGF-I and HGF, by cell proliferation (G; *p<0.05 vs. base, +p<0.05 vs. CTRL, Jp<0.05 vs. HGF) and migration (H; +p<0.05 vs. CTRL, Jp<0.05 vs. IGF-I). (I) Western blot analysis revealed that upon ligand binding specific downstream effector pathways are activated in c-kit^{pos} porcine cardiac cells, phos=phosphorylated, FAK=focal adhesion kinase.

FIG. 3 Intra-Coronary Injection of IGF-I and HGF Improves Myocardial Cell Remodeling after AMI.

(A) H&E staining of GF-treated pig hearts revealed islands of survived myocardial tissue in the infarct zone (arrows), disposed between the regenerating and fibrotic layers. (B) These myocardial islands were infrequent and less defined in structure in the saline-treated CTRL pig hearts. (C) These myocardial islands were composed of mainly BrdU negative cardiomyocytes (cardiac troponin I, cTn1; grey with their nuclei as black circles in the middle of the cell), documenting their survived and mature phenotype. The cells born after the infarct are BrdU positive and their nuclei show as white dots. (D-E) Sirius red staining identified fibrotic tissue (grey staining) and muscle (yellow staining) in cross sections of the infarct zone, in GF-treated (D) and saline-treated CTRL (E) pig hearts. (F) GF-treated (IGF-1/HGF) pig hearts had a decreased percentage area fraction of fibrosis in the infarct zone, compared to saline-treated CTRL pigs. *p<0.05 vs. CTRL. +p<0.05 vs. IGF-1/HGF I×. (G) Staining for activated caspase-3 (brown; arrowheads) revealed apoptotic myocytes in the peri-infarct/border zone of the CTRL pig heart after AMI. (H) IGF-I and HGF injection resulted in decreased numbers of apoptotic myocytes, in the peri-infarct/border zone, compared to saline-treated CTRL. *p<0.05, vs. CTRL, +p<0.05 vs. IGF-1/HGF I×, $p<0.05 vs. IGF-1/HGF 2×. (I) Analysis of myocyte diameter showed that GF-treated pigs had a decreased myocyte hypertrophic response after AMI, when compared to saline treated CTRL animals. Normal=remote/distal region from infarcted area in CTRL hearts. ̂p<0.05 vs. Normal, *p<0.05 vs. CTRL. +p<0.05 vs. IGF-1/HGF I×.

FIG. 4 IGF-I and HGF Administration after AMI Activates Endogenous CSCs, Driving their Commitment to the Cardiac Lineage

(A-B) The majority of porcine ckit^{pos} CSCs (white) express Igf-1 (A, grey) and c-met (B, grey) receptors in vivo. DAPI stains the nuclei in blue. (C) A cluster of ckit^{pos} CSCs (white) in the area of infarct of a GF-4× treated pig heart. (D) The number of c-kit^{pos} CSCs significantly increased in the border but more in the infarcted region of GF-treated pigs, compared to saline-treated CTRL. *p<0.05, vs. CTRL, +p<0.05 vs. IGF-1/HGF I×, Jp<0.05 vs. IGF-1/HGF 2×. (E) Many c-kit^{pos} CSCs (white) in the GF-treated pig hearts were positive for BrdU (grey), indicative of their newly formed status. (F) c-kit^{pos} CSCs (white) expressed the cardiac transcription factor, Nkx2.5 (grey), representing cardiac progenitor cells. Nuclei were stained with DAPI (blue). (G) The number of c-kit^{pos}Nkx2.5^{pos} cardiac progenitor cells increased in the infarct and border zones in GF-treated pig hearts, *p<0.05, vs. CTRL, +p<0.05 vs. IGF-1/HGF Ix, Jp<0.05 vs. IGF-1/HGF 2×. (H-I) Some c-kit^{pos} CSCs (white) expressed the transcription factors, GAT A6 (H; grey) and Ets-1 (I; grey), indicative of smooth muscle and endothelial cell differentiation, respectively.

FIG. 5. IGF-1/HGF Intracoronary Administration Induces Substantial New Myocyte Formation after AMI.

(A-B) Regenerating bands of small, newly formed BrdU^{pos} (WHITE) MYOCYTES (GREY; α-SARCOMERIC actin, α-Sarc Act) in the infarct regions of GF-I× (A) and GF-4× (B) treated pig hearts. Note the increased size of the regenerating band after 4× the amount of GF administration. Also the myocytes are more dense, compact and structured as myocardium after 4× the amount of GF administration. (C) Within these regenerating bands in the infarct zone were small Ki67^{pos} (white) proliferating myocytes (grey; α-Sarc Act). (D-E) Newly formed small BrdU^{pos} (white nuclei) myocytes (grey; α-Sarc Act cytoplasm) in the border zone after GF-I× (D) and GF-4× (E) doses. (F) Small Ki67^{pos} (white) myocytes (grey α-Sarc Act) were also present in the border zone after GF-injection. (G-H) The fraction of small BrdU^{pos} and Ki67^{pos} myocytes significantly increased in the border but more in the infarct region after GF injection. *p<0.05, vs. CTRL, +p<0.05 vs. IGF-1/HGF I×, $p<0.05 vs. IGF-1/HGF 2×. (I) A small Ki67^{pos} mitotic myocyte in the infarct zone of a GF-4× treated pig heart.

FIG. 6 Growth Factor Administration Increased the Generation of New Vascular Structures and Improved Cardiac Function in the Infarcted Pig Heart.

(A) Newly formed arterial structures (BrdU, white; α-smooth muscle actin, SMA, white; Myosin Heavy Chain, MHC, grey; DAPI, blue) were evident in the infarcted region of GF-treated pig hearts. (B-C) Newly formed capillaries were also evident in the infarcted regions after IGF-I and HGF injection (BrdU, white; vWF, grey; DAPI, dark grey). (D-F) The number of capillaries in GF-treated pigs was significantly increased in the infarct zone, compared to saline treated (dark grey stain) CTRL. *p<0.05 vs. CTRL, +p<0.05 vs. IGF-1/HGF I×, Jp<0.05 vs. IGF-1/HGF 2×. Images (2O× magnification) show vWF staining (dark grey) in saline-treated CTRL (D) and GF-4× (E) treated hearts. Capillaries were defined as vessels composed of 1 or 2 endothelial cells. (G-H) GF-treated hearts showed improved left ventricular (LV) ejection fraction (G) and radial strain (H), compared to saline-treated CTRL. *p<0.05 vs. Baseline, #p<0.05 vs. AMI, +p<0.05 vs. CTRL, }p<0.05 vs. GF-I×. (I) Representative Tissue Doppler radial strain tracing from CTRL (a-c) and GF-4× (d-f) treated pigs. CTRL (b) and GF-4× (e) treated pigs had equal de-synchronization of antero-septal contraction following 90 minutes of coronary occlusion (AMI). At sacrifice (Post-MI), de-synchronized contraction worsened in CTRL (c) while it was improved in GF-treated (f) pigs.

The results shown above demonstrate that microgram doses of these growth factors improve myocardial remodeling, foster the activation of the resident CSCs, which produce extensive new myocardial formation, improving LV function in a dose dependent manner in an animal heart of size and anatomy similar to the human using a clinically implementable protocol. Thus, IGF-1/HGF injection produced a wide variety of beneficial effects on cardiac remodeling and autologous cell regeneration that were proportional to the dose of GF administered.

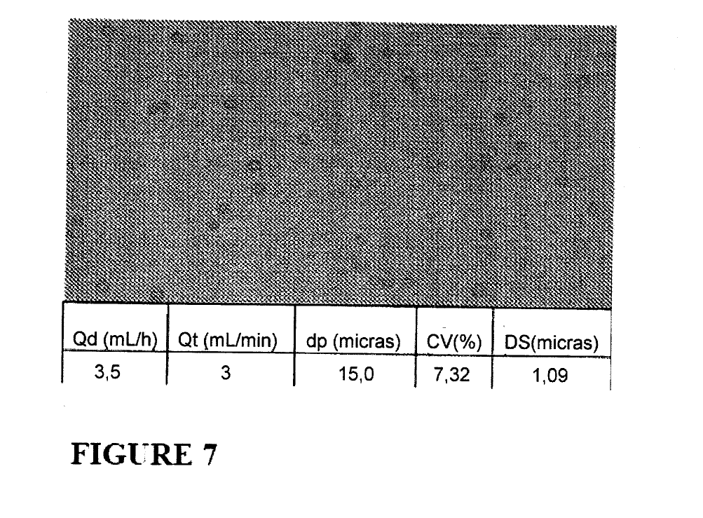

FIG. 7 shows Optical microscope image of the PLGA particles containing IGF-1 obtained with the recipe described above

FIG. 8 shows an electron micrograph of the same batch of particles shown in the figure above.

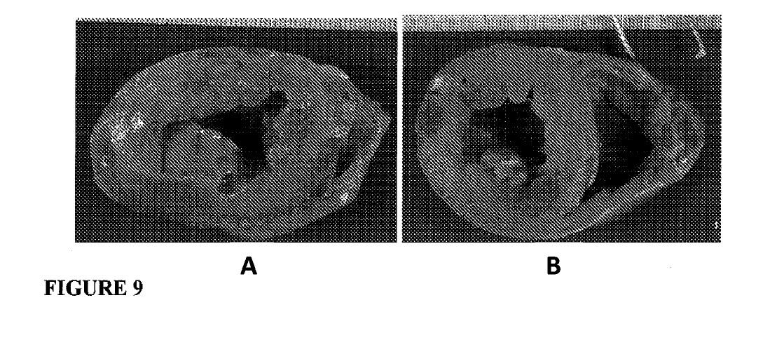

FIG. 9 shows sections of the hearts of pig #1 (left image) and pig #2 (right image). The anterior wall of the left ventricle, irrigated by the left coronary artery, of pig #1 shows a number of micro infarcts (paler areas), while the myocardium of pig #2 is normal as shown by the uniform coloration.

FIG. 10A. Sections of the myocardium of pig #3, sacrificed 30 min after the administration of a mixture of polystyrene (red beads-shown in the figure as grey, larger diameter, smooth circles) and PLGA+growth factors (green beads-shown in the figure as white, smaller diameter and more irregular shape) beads. The appearance difference in size between the red and green particles is due to the higher fluorescence of the red

FIGS. 10B and 10C show sections of the myocardium of pig #4, sacrificed 24 hours after the administration of a mixture of polystyrene (red—shown in figures as grey, larger diameter, smooth circles) and PLGA+growth factors (green—shown in the figure as white, smaller diameter and more irregular shape) beads. The ratio of green to red beads is significantlo lower in this animal because of the degradation of the PLGA microparticles In the four panel of the left only red beads are detected, while in those of the right the ratio is closer to 1:1.

FIG. 11 shows Microscopic sections of two areas of pig #4. Myocytes are in grey. Nuclei in darker gry. The endogenous cardiac stem cells (CSCs) are identified by an arrow head (upper) and an arrow (lower). Their membrane is labeled in paler green. On the upper figure, the nuclei are clean because the cells are quiescent. On the lower figure all the CSCs have pale grey stain in the nuclei that identifies the protein Ki-67 a marker of cells that have entered the cell cycle.

FIG. 12. Local Administration of IGF-I and HGF Encapsulated into 15 μm PLGA Microspheres Enhances the Regeneration of Damaged Skeletal Muscle.

Histological images of control and damaged quadriceps muscle. Panel A: Histological image of the left muscle (control) five days after producing the lesion on the right muscle. No treatment was administered to this leg. Panel B: Histological section of a right quadriceps five days after producing the damage with no treatment (damaged control). The arrowheads point to two of the several extensive areas of cell necrosis where a concentration of nuclei appear to initiate a regenerative reaction. Panel C: Right biopsy of right quadriceps 3 days after the lesion treated with a mixture of microspheres loaded with IGF-I and microspheres loaded with HGF with a total administered equivalent of 16 μg IGF-I and 4 μg HGF. The arrow heads point toward young micro fibers in the damaged areas in a very process of regeneration. Panel D: Biopsy of the same muscle shown in Panel C two days later (5 days after the lesion). The smaller sized dark fibers are regenerated fibers labeled with an antibody against embryonic myosin heavy chain, a marker or regenerated fibers. The image in this panel is the equivalent to the one in Panel B. The striking difference between the two images shows the effectiveness of the therapy.

FIG. 13. Enhanced Myocardial Regenerative Capacity of the Combination of IGF-1/HGF/SCF Administered Intracoronary Encapsulated in PLGA Microspheres of 15 μm in Diameter

The bar graph of FIG. 13A compares the effect in the number of regenerated cardiac myocytes in pigs post-AMI treated with a combination of two types of microspheres, white bars (one loaded with IGF-I and the other with HGF) with the animals treated with a combination of three types of microspheres (hrIGF-1, hrHGF, and hrSCF), black bars. It is obvious that at the three different concentrations used the combination of 3 types of microspheres each loaded with a different factor is superior to the combination of only two. CTRL=control animals treated with placebo; White bars: IX animals administered microspheres loaded with the equivalent of 2 μg IGF-I and 0.5 μg HGF biologically active; 2×=4 μg IGF-I and 1 μg HGF and 4× dose=8 μg of IGF-I and 2 μg of HGF. Black bars: Same amounts of IGF-I and HGF as for the animals represented by the white bars plus microspheres loaded with SCF equivalent to 2, 4 and 8 μg of biologically active hrSCF

FIG. 13B Shows the Left Ventricle Ejection Fraction Prior to, Immediately after and 4 Weeks Post-AMI as Determined by Echocardiography of the Pigs Treated with Different Combinations of Microspheres.

Baseline=LV ejection fraction just prior the AMI; AMI=LV ejection fraction after AMI; Post-AMI=LV ejection fraction 4 weeks after AMI and local GF treatment. C=Control animals treated with placebo post-AMI; O=animals treated with 4× dose of IGF-I+HGF in solution intracoronary; ▾=animals treated with a 4× dose of IGF-I+HGF encapsulated in PLGA microspheres administered just downstream to the site of coronary occlusion; Δ=animals treated with a 4× dose of IGF-1+HGF+SCF each separately encapsulated in PLGA microspheres administered just downstream to the site of coronary occlusion.

FIG. 14. Effects of the UPy Hydrogel Carrier on IGF-1/HGF Release and Bioactivity In Vitro

(A) Schematic study design, showing the targeted intramyocardial delivery in the MI borderzone of 1) empty UPy-hydrogel as control (CTRL), 2) IGF-1/HGF dissolved in saline, denoted as GF, or 3) UPy-hydrogel with IGF-1/HGF, denoted as UPy-GF. (B) ELISA essay showing that the release of IGF-1 and HGF from UPy-hydrogel was sustained over a four-day period. (C) Western blot showing that IGF-1/HGF, released from the UPy-hydrogel, was able to activate their corresponding receptors on HeLa cells.

FIG. 15. UPy-IGF-1/HGF Therapy Improves Cardiac Function in Chronic MI

(A to B) 2-dimensional echocardiography (2DE) images showing change in fractional area shortening (FAS) for (A) controls and (B) UPy-GF treated animals. Cumulative data of various parameters of LV systolic function, such as (C) LVEF measured by real-time 3D echocardiography (RT3DE) (D,E) RT3DE derived end diastolic and end systolic volumes and (F) preload recruitable stroke work (PRSW) measured by intracardiac pressure-volume loop recordings. (G). FAS measured by 2DE and (H) Diastolic function measured by 2DE. * denotes p<0.05 vs CTRL. All data are mean±SD, n=3, 5, 5 for CTRL, GF and UPy-GF respectively.

FIG. 16(A-J). IGF-1/HGF Treatment Reduced Pathological Hypertrophy in the MI Borderzone

(A,B) Representative MI borderzone sections (hematoxylin and eosin (H&E) staining) showing adverse cardiac hypertrophy in the control treated animals (A), which was not observed in the UPy-GF treated animals (B). (C to H) Picric Sirius red staining in bright field images (C to E) and under polarized light (F to H) showing extensive scar tissue in all groups depicted as red staining in bright field microscopy. Under polarized light, color depended on the collagen fiber density (yellow for higher intensity, green for lower intensity). In both growth factor treated groups, small myocardial islands were visible in the infarct area (see arrowheads). Quantification of (I) cardiomyocyte diameter in the MI borderzone and (J) fibrosis. * denotes p<0.05 vs CTRL. All data are mean±SD, n=3, 4, 5 for CTRL, GF and UPy-GF respectively. MI denotes myocardial infarction.

FIG. 17(A-E). IGF-1/HGF Administration Leads to Formation of New Cardiac Myocytes

(A to B) Expression of cellular proliferation marker Ki67 (green) showed increased proliferation index of cells (arrowheads) in the UPy-GF treated animals, compared to CTRL. (C to D) Increased newly formed Ki67^{pos} (green) cardiomyocytes (arrowheads, asterix, see inset) after GF treatment, compared to CTRL in the peri-infarct/borderzone. (E) Ki67^{pos} cardiac myocytes were smaller than the quiescent Ki67^{neg} cardiomyocyte fraction, indicative of their immature, newly formed nature. * denotes p<0.05 vs CTRL. † denotes p<0.05 vs Ki67^{neg} cardiac myocytes. All data are mean±SD, n=3, 4, 5 for CTRL, GF and UPy-GF respectively.

FIG. 18(A-C). IGF-1/HGF Leads to Increased Capillerisation and Reduces Microvascular Resistance

(A) Staining for Von Willebrand factor (vWF) show small capillary structures (red arrowheads, asterix, see inset) in the borderzone of the UPy-GF treated heart. (B) Number of capillaries in the peri-infarct/borderzone area. (C) Relative change, compared to baseline, in simultaneously measured intracoronary pressure and flow derived hyperemic microvascular resistance (HMR). * denotes p<0.05 vs CTRL. All data are mean±SD, n=3, 4, 5 for CTRL, GF and UPy-GF respectively.

FIG. 19(A-F). IGF-1/HGF Treatment Increases the epCSC Compartment and Drives their Cardiac Commitment in Chronic MI

(A) The infarct area harbors various cell types, such as i) c-kit^{pos} CD45^{neg} epCSCs, ii) c-kit^{neg} CD45^{pos} cells or iii) c-kit^{pos} CD45^{pos} cells (including mast cells). (B) Endogenous epCSCs were a morphologically distinct subset of small cells showing perinuclear expression of c-kit (green)(arrowheads) and negative for CD45. (C) Quantification of epCSCs in the peri-infarct/border and infarct zone. (D) A c-kit^{pos} (green) myogenic progenitor (arrowhead, asterix, see inset), expressing the early cardiac transcription factor, Nkx2.5 (white). (E) Quantification of Nkx2.5^{pos} epCSCs in the peri-infarct/border and infarct zone. * denotes p<0.05 vs CTRL. All data are mean±SD, n=3, 4, 5 for CTRL, GF and UPy-GF respectively. (F) Some c-kit^{pos} epCSCs also expressed the transcription factor ETS-1 (arrowhead, asterix, see inset).

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

Embodiments of the disclosure are hereby described as comprising integers. The disclosure also extends to separate embodiments consisting of or consisting essentially of said integers.

It is also specifically envisages that the disclosure extends to combinations of one or more embodiments described herein, where technically feasible.

All patents and patent applications referred to herein are incorporated by reference in their entirety. The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the claims.

1. A pharmaceutical formulation for parenteral administration to a target tissue comprising particles containing an active ingredient and a biodegradable excipient, wherein 90% or more of the particles have a diameter of between 10 and 20 microns and the formulation is substantially free of particles with a diameter greater than 50 microns and less than 5 microns, such that where the formulation is administered upstream of the target tissue the ability of the active to pass through the target tissue and pass into systemic circulation is restricted.

2. A pharmaceutical formulation according to claim 1, which is substantially free of particles with a diameter greater than 20 microns and less than 5 microns.

3. A pharmaceutical formulation according to claim 1, wherein at least 90% of the particles have a diameter that is between 15 and 20 microns.

4. A pharmaceutical composition according to claim 1 wherein at least 95%, at least 98% or at least 99% of the particles have a diameter of between 10 and 20 microns.

5. A pharmaceutical composition according to claim 3 wherein at least 95%, at least 98% or at least 99% of the particles have a diameter of between 15 and 20 microns.

6. A pharmaceutical composition according to claim 1 wherein the size of the particles is monodispersed.

7. A pharmaceutical composition according to claim 6 wherein at least 68% of particles have a size +/−1 micron of the mean particle size.

8. A pharmaceutical composition according to claim 7 wherein at least 99% of particles have a size +/−1 micron of the mean particle size.

9. A pharmaceutical composition according to claim 1, wherein the particles have a mean particle size of 15 microns.

10. A pharmaceutical composition according to claim 1 for parenteral administration to an ischemic tissue.

11. A pharmaceutical composition according to claim 10 for parenteral administration to a cardiac ischemic tissue.

12. A pharmaceutical formulation according to claim 11 which further comprises a growth factors selected from: HGF (hepatocyte growth factor); IGF (insulin-like growth factor) such as IGF-I; PDGF (Platelet-derived growth factor) such as PDGF-β, FGF (fibroblast growth factor) such as aFGF (FGF-I) or bFGF (FGF-2) and FGF-4; SDF-I (stromal cell-derived factor 1); EGF (epidermal growth factor); VEGF (vascular endothelial growth factor); erythropoietin (EPO); TGF β(transforming growth factor β); G-CSF (Granulocyte-colony stimulating factor); GM-CSF (Granulocyte-macrophage colony stimulating factor), Bone morphogenetic proteins (BMPs, BMP-2, BMP-4); Activin A; IL-6; Neurotrophins for example NGF (Nerve growth factor), BDNF (brain-derived neurotrophic factor), NT-3 (neurotrophin-3), NT-4 (neurotrophin-4) and (neurotrophin-1), which is structurally unrelated to NGF, BDNF, NT-3 and NT-4; TPO (Thrombopoietin); GDF-8 (Myostatin); GDF9 (Growth differentiation factor-9); Periostin, Wint3A or Neuroregulin.

13. A pharmaceutical composition according to claim 1 containing an active ingredient selected from the group comprising HGF and/or IGF.

14. A pharmaceutical composition according to claim 13 which further comprises PDGF (Platelet-derived growth factor) such as PDGF-β, FGF (fibroblast growth factor) such as aFGF (FGF-I) or bFGF (FGF-2) and FGF-4; SDF-I (stromal cell-derived factor 1); EGF (epidermal growth factor); VEGF (vascular endothelial growth factor); erythropoietin (EPO); TGF β (transforming growth factor β); G-CSF (Granulocyte-colony stimulating factor); GM-CSF (Granulocyte-macrophage colony stimulating factor), Bone morphogenetic proteins (BMPs, BMP-2, BMP-4); Activin A; IL-6; Neurotrophins for example NGF (Nerve growth factor), BDNF (brain-derived neurotrophic factor), NT-3 (neurotrophin-3), NT-4 (neurotrophin-4) and (neurotrophin-1), which is structurally unrelated to NGF, BDNF, NT-3 and NT-4; TPO (Thrombopoietin); GDF-8 (Myostatin); GDF9 (Growth differentiation factor-9); Periostin, Wint3A or Neuroregulin.

15. A pharmaceutical composition according to claim 14 which further comprises SCF-I.

16. A pharmaceutical composition according to claim 1, wherein the concentration of the active ingredient is in the range of 1 ng per 1×10⁶ particles up to 4 mg per 1×10⁶ microspheres.

17. A pharmaceutical formulation according to claim 1, wherein at least 30% of the active ingredient is retained in the target tissue after administration.

18. A pharmaceutical formulation according to claim 17, wherein at least 40%, at least 50%, at least 60%, at least 70% such as at least 80% of the active ingredient is retained.

19. A pharmaceutical composition according to claim 1, wherein the parenteral administration is intra-arterial administration.

20. A pharmaceutical formulation as defined in claim 1, for treatment.

21. A pharmaceutical formulation as defined in claim 1, for targeting a selected tissue or organ.

22. A pharmaceutical formulation according to claim 21, wherein the organ is selected from the heart, lung(s), liver, kidney(s), bladder, uterus, testis, pancreas, spleen or intestines.

23. A pharmaceutical formulation according to claim 22, for targeting cardiac tissue.

24. A pharmaceutical formulation according claim 23, for the treatment of myocardial infarction (MI) acute or chronic, ischemic heart disease, with or without a myocardial infarction

25. A method of localized delivery comprising the step of administering into the circulation upstream of cardiac tissue a pharmaceutical composition as defined in claim 1.

26. A method according to claim 25 wherein the localized delivery is through is intra-arterial administration.

27. Use of HGF or IGF-I for regeneration in cardiac tissue by stimulating stems cells resident in mature cardiac tissue.

28. Use of a growth factor as defined in claim 12 for inducing cellular protection of cardiac tissue-specific stem cells from ischemic damage and reducing their death by apoptosis and/or necrosis

29. Use of a growth factor as defined in claim 12, for stimulating Oct4-expressing stern cells.

30. Use of a growth factor as defined in claim 12 wherein cardiac tissue-specific stem cells are also stimulated.

31. A pharmaceutical formulation according to claim 1, for the treatment of cerebral vascular accident (stroke).

32. A pharmaceutical formulation according to claim 1 for the treatment of any cell loss produced as a consequence of reduced blood flow (ischemia) or degenerative disease in any other tissue.

33. A pharmaceutical composition according to claim 1 wherein the pharmaceutical composition comprises a mixed population of particles, said population comprising particles having a first active ingredient in admixture with particles having one or more further distinct active ingredients.

34. A composition comprising an injectable supramolecular hydrogel and one or more growth factors, wherein the hydrogel is ureido-pyrimidinone (UPy) and the growth factor is insulin-like growth factor-1 (IGF-1) and hepatocyte growth factor (HGF).

35. The composition of claim 34 comprising 0.1-0.4 μg, 0.4-0.8 μg, 0.8-1 μg, 1-2 μg, 2-4 μg, 4-8 μg or 8-10 μg of growth factor per ml of UPy hydrogel.

36. The composition of claim 34 wherein the composition is a pharmaceutical composition for parenteral administration to a cardiac ischemic tissue.

37. A method of treating a patient by administration of the composition of claim 34 wherein the patient has myocardial infarction.

38. The method of claim 37 comprising a targeted intramyocardial injection of a composition.

39. The method of claim 38 wherein the intramyocardial injection targets the border zone of infarct scar.

40. A method of treating chronic myocardial infarction using the composition of claim 34 wherein the treatment activates c-kit^{pos}, CD45^{neg}, and/or epCSCs.

41. The method of claim 40 wherein the treatment increases c-kit^{pos}, CD45^{neg}, and/or epCSCs population by four fold in the borderzone of treated hearts as compared to non-treated hearts.

42. A method of treatment using the composition of claim 34 wherein the treatment reduces pathological cardiac hypertrophy, increases epCSC number and formation of new cardiomyocytes and capillaries.

43. A method of treatment using the composition of claim 34 wherein the treatment enhances myocardial repair and regeneration in the acute phase of myocardial infarction.

44. The method of treatment of claim 37, wherein the patient is not treated with composition comprising microspheres.

45. A composition according to claim 36 wherein the parenteral administration is intra-arterial administration.

46. A method of treating a patient by administering a composition of claim 37 wherein the method targets a selected tissue or organ.

47. The method of claim 46 wherein the organ is heart, lung, liver, kidney, bladder, uterus, testis, pancreas, spleen or intestines.

48. The method of claim 46 wherein the method targets cardiac tissues.

49. The method of claim 48 for the treatment of cerebral vascular accident (stroke), myocardial infarction (MI) acute or chronic, ischemic heart disease, with or without a myocardial infarction.

50. The method of claim 46 for the treatment of cell loss as a consequence of reduced blood flow (ischemia) or degenerative disease.
